SEQUENCE LISTING (101 sequences)

SEQ ID NO: 1 - DNA, 189 bp, synthetic construct
  source 1..189: mol_type = other DNA
cgccagcagtggcaatagcgtttccggccttttgtgccgggagggtcggcgagtcgctgacttaacgccagtagtatgtccatatacccaaagtcgcttcattgtacctgagtacgcttcgcgtgcgctgacgcgctcagtacagttacgcgccttcgggatggtttaatggtattgccgctgttggcg

SEQ ID NO: 2 - DNA, 268 bp, synthetic construct
  source 1..268: mol_type = other DNA
gcgcgagcagccgagagaggtccggagtgcatcagcctgagcgcctcgagcggcggagcggcgttgcgccgctccggttggaatgcaggacactctccgcaaggtagcctgttcttggctctctccctcctaggcactacggccagggtgggtagcggccgccgtttacccaccccggccgtagtgcctaggaggggagagccggtgaggctaccgtgcccccaggtaagatggtggtgctttcccggcctccctcgactgctcgcgc

SEQ ID NO: 3 - DNA, 82 bp, synthetic construct
  source 1..82: mol_type = other DNA
atgcgcacccttagcgagaggtttatcattaaggtcaacctctggatgttgtttcggcatcctgcattgaatctgagttact

SEQ ID NO: 4 - DNA, 59 bp, synthetic construct
  source 1..59: mol_type = other DNA
cgccagtagtatgtccatatacccaaagtcgcttcattgtacctgagtacgcttcgcgt

SEQ ID NO: 5 - DNA, 42 bp, synthetic construct
  source 1..42: mol_type = other DNA
cgccagtagtatgtccatatacccaaagtcgcttcattgtac

SEQ ID NO: 6 - DNA, 24 bp, synthetic construct
  source 1..24: mol_type = other DNA
cgccagtagtatgtccatgaattc

SEQ ID NO: 7-8 - skipped (2 entries)

SEQ ID NO: 9 - DNA, 48 bp, synthetic construct
  source 1..48: mol_type = other DNA
gcgctgacgcgctcagtacagttacgcgccttcgggatggtttaatgg

SEQ ID NO: 10 - DNA, 33 bp, synthetic construct
  source 1..33: mol_type = other DNA
gtacagttacgcgccttcgggatggtttaatgg

SEQ ID NO: 11 - DNA, 18 bp, synthetic construct
  source 1..18: mol_type = other DNA
gaattcatggtttaatgg

SEQ ID NO: 12-13 - skipped (2 entries)

SEQ ID NO: 14 - DNA, 86 bp, synthetic construct
  source 1..86: mol_type = other DNA
tccggttggaatgcaggacactctccgcaaggtagcctgttcttggctctctccctcctaggcactacggccagggtgggtagcgg

SEQ ID NO: 15 - DNA, 82 bp, synthetic construct
  source 1..82: mol_type = other DNA
tccggttggaatgcaggacactctccgcaaggtagcctgttcttggctctctccctcctaggcactacggccagggtgggta

SEQ ID NO: 16 - DNA, 51 bp, synthetic construct
  source 1..51: mol_type = other DNA
tccggttggaatgcaggacactctccgcaaggtagcctgttcttggctctc

SEQ ID NO: 17 - DNA, 37 bp, synthetic construct
  source 1..37: mol_type = other DNA
tccggttggaatgcaggacactctccgcaaggtagcc

SEQ ID NO: 18 - DNA, 30 bp, synthetic construct
  source 1..30: mol_type = other DNA
tccggttggaatgcaggacactctccgcaa

SEQ ID NO: 19 - DNA, 76 bp, synthetic construct
  source 1..76: mol_type = other DNA
ccgccgtttacccaccccggccgtagtgcctaggaggggagagccggtgaggctaccgtgcccccaggtaagatgg

SEQ ID NO: 20 - DNA, 68 bp, synthetic construct
  source 1..68: mol_type = other DNA
tacccaccccggccgtagtgcctaggaggggagagccggtgaggctaccgtgcccccaggtaagatgg

SEQ ID NO: 21 - DNA, 38 bp, synthetic construct
  source 1..38: mol_type = other DNA
gagagccggtgaggctaccgtgcccccaggtaagatgg

SEQ ID NO: 22 - DNA, 26 bp, synthetic construct
  source 1..26: mol_type = other DNA
ggctaccgtgcccccaggtaagatgg

SEQ ID NO: 23 - DNA, 19 bp, synthetic construct
  source 1..19: mol_type = other DNA
gtgcccccaggtaagatgg

SEQ ID NO: 24 - DNA, 124 bp, synthetic construct
  source 1..124: mol_type = other DNA
cgccagcagtggcaatagcgtttccggccttttgtgccgggagggtcggcgagtcgctgacttaacgccagtagtatgtccatatacccaaagtcgcttcattgtacctgagtacgcttcgcgt

SEQ ID NO: 25 - DNA, 107 bp, synthetic construct
  source 1..107: mol_type = other DNA
cgccagcagtggcaatagcgtttccggccttttgtgccgggagggtcggcgagtcgctgacttaacgccagtagtatgtccatatacccaaagtcgcttcattgtac

SEQ ID NO: 26 - DNA, 89 bp, synthetic construct
  source 1..89: mol_type = other DNA
cgccagcagtggcaatagcgtttccggccttttgtgccgggagggtcggcgagtcgctgacttaacgccagtagtatgtccatgaattc

SEQ ID NO: 27 - DNA, 73 bp, synthetic construct
  source 1..73: mol_type = other DNA
cgccagcagtggcaatagcgtttccggccttttgtgccgggagggtcggcgagtcgctgacttaacgccagta

SEQ ID NO: 28 - DNA, 71 bp, synthetic construct
  source 1..71: mol_type = other DNA
cgccagcagtggcaatagcgtttccggccttttgtgccgggagggtcggcgagtcgctgacttaacgccag

SEQ ID NO: 29 - DNA, 158 bp, synthetic construct
  source 1..158: mol_type = other DNA
gcgcgagcagccgagagaggtccggagtgcatcagcctgagcgcctcgagcggcggagcggcgttgcgccgctccggttggaatgcaggacactctccgcaaggtagcctgttcttggctctctccctcctaggcactacggccagggtgggtagcgg

SEQ ID NO: 30 - DNA, 154 bp, synthetic construct
  source 1..154: mol_type = other DNA
gcgcgagcagccgagagaggtccggagtgcatcagcctgagcgcctcgagcggcggagcggcgttgcgccgctccggttggaatgcaggacactctccgcaaggtagcctgttcttggctctctccctcctaggcactacggccagggtgggta

SEQ ID NO: 31 - DNA, 123 bp, synthetic construct
  source 1..123: mol_type = other DNA
gcgcgagcagccgagagaggtccggagtgcatcagcctgagcgcctcgagcggcggagcggcgttgcgccgctccggttggaatgcaggacactctccgcaaggtagcctgttcttggctctc

SEQ ID NO: 32 - DNA, 109 bp, synthetic construct
  source 1..109: mol_type = other DNA
gcgcgagcagccgagagaggtccggagtgcatcagcctgagcgcctcgagcggcggagcggcgttgcgccgctccggttggaatgcaggacactctccgcaaggtagcc

SEQ ID NO: 33 - DNA, 102 bp, synthetic construct
  source 1..102: mol_type = other DNA
gcgcgagcagccgagagaggtccggagtgcatcagcctgagcgcctcgagcggcggagcggcgttgcgccgctccggttggaatgcaggacactctccgcaa

SEQ ID NO: 34 - DNA, 65 bp, synthetic construct
  source 1..65: mol_type = other DNA
gcgctgacgcgctcagtacagttacgcgccttcgggatggtttaatggtattgccgctgttggcg

SEQ ID NO: 35 - DNA, 50 bp, synthetic construct
  source 1..50: mol_type = other DNA
gtacagttacgcgccttcgggatggtttaatggtattgccgctgttggcg

SEQ ID NO: 36 - DNA, 35 bp, synthetic construct
  source 1..35: mol_type = other DNA
gaattcatggtttaatggtattgccgctgttggcg

SEQ ID NO: 37 - DNA, 23 bp, synthetic construct
  source 1..23: mol_type = other DNA
taatggtattgccgctgttggcg

SEQ ID NO: 38 - DNA, 21 bp, synthetic construct
  source 1..21: mol_type = other DNA
atggtattgccgctgttggcg

SEQ ID NO: 39 - DNA, 110 bp, synthetic construct
  source 1..110: mol_type = other DNA
ccgccgtttacccaccccggccgtagtgcctaggaggggagagccggtgaggctaccgtgcccccaggtaagatggtggtgctttcccggcctccctcgactgctcgcgc

SEQ ID NO: 40 - DNA, 102 bp, synthetic construct
  source 1..102: mol_type = other DNA
tacccaccccggccgtagtgcctaggaggggagagccggtgaggctaccgtgcccccaggtaagatggtggtgctttcccggcctccctcgactgctcgcgc

SEQ ID NO: 41 - DNA, 72 bp, synthetic construct
  source 1..72: mol_type = other DNA
gagagccggtgaggctaccgtgcccccaggtaagatggtggtgctttcccggcctccctcgactgctcgcgc

SEQ ID NO: 42 - DNA, 60 bp, synthetic construct
  source 1..60: mol_type = other DNA
ggctaccgtgcccccaggtaagatggtggtgctttcccggcctccctcgactgctcgcgc

SEQ ID NO: 43 - DNA, 52 bp, synthetic construct
  source 1..52: mol_type = other DNA
gtgcccccaggtaagatggtggtgctttcccggcctccctcgactgctcgcg

SEQ ID NO: 44 - protein, 319 aa, synthetic construct
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 45 - protein, 315 aa, synthetic construct
RIYSLIDSQTLMTKGFASEVMRSPEPPKKWDIAKKKGGMRTIYHPSSKVKLIQYWLMNNVFSKLPMHNAAYAFVKNRSIKSNALLHAESKNKYYVKIDLKDFFPSIKFTDFEYAFTRYRDRIEFTTEYDKELLQLIKTICFISDSTLPIGFPTSPLIANFVARELDEKLTQKLNAIDKLNATYTRYADDIIVSTNMKGASKLILDCFKRTMKEIGPDFKINIKKFKICSASGGSIVVTGLKVCHDFHITLHRSMKDKIRLHLSLLSKGILKDEDHNKLSGYIAYAKDIDPHFYTKLNRKYFQEIKWIQNLHNKVE

SEQ ID NO: 46 - protein, 406 aa, synthetic construct
GRPYVTLNLNGMFMDKFKPYSKSNAPITTLEKLSKALSISVEELKAIAELPLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHDLPINKRKTKIFHCSSEPIKVHGLRVDYDSPRLPSDEVKRIRASIHNLKLLAAKNNTKTSVAYRKEFNRCMGRVNKLGRVAHEKYESFKKQLQAIKPMPSKRDVAVIDAAIKSLELSYSKGNQNKHWYKRKYDLTRYKMIILTRSESFKEKLECFKSRLASLKP

SEQ ID NO: 47 - protein, 318 aa, synthetic construct
DATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAW

SEQ ID NO: 48 - protein, 479 aa, synthetic construct
TAKLESHVPAAPPVSAEAPAPTRPDAAKQEARRAHHEALRLRWKAIEEAGGTDAWVRQQLVAKGVAAEEVDFESLSDKQKAAWKEKKKAEATERRAQKRLAWEAWKATHIHHLGVGVHWDEAGGPDKFDVAGREERAKANGLPEGLDSVEALAKALGISVSRLRWFSFHREVDTGTHYQTWEIPKRDGGKRTLTAPKRELKAVQRWVLANVVERLPVHGAAHGFVAGRSILTNALAHQGADVVVKVDMKDFFPSVTWPRVKGLLRKGGLPENLATLLALLSTEAPREVVRFRGETLYVAKGPRALPQGAPTSPALTNALCLRLDKRLSALSKRLGFTYTRYADDLTFSWRRAKKSRQKELPLADAPVALLLARVKGVLEAEGFTLHPDKTRVQRKGSRQRVTGLVVNEAPEGVPGARVPRDVVRRLRAAIHNREQGKPGPTGETLEQLKGLAAFLHMTDAEKGRAFLRRLEALEKRQTA

SEQ ID NO: 49 - DNA, 59 bp, synthetic construct
  source 1..59: mol_type = other DNA
acgcgaagcgtactcaggtacaatgaagcgactttgggtatatggacatactactggcg

SEQ ID NO: 50 - DNA, 42 bp, synthetic construct
  source 1..42: mol_type = other DNA
gtacaatgaagcgactttgggtatatggacatactactggcg

SEQ ID NO: 51 - DNA, 24 bp, synthetic construct
  source 1..24: mol_type = other DNA
gaattcatggacatactactggcg

SEQ ID NO: 52-53 - skipped (2 entries)

SEQ ID NO: 54 - DNA, 86 bp, synthetic construct
  source 1..86: mol_type = other DNA
ccgctacccaccctggccgtagtgcctaggagggagagagccaagaacaggctaccttgcggagagtgtcctgcattccaaccgga

SEQ ID NO: 55 - DNA, 82 bp, synthetic construct
  source 1..82: mol_type = other DNA
tacccaccctggccgtagtgcctaggagggagagagccaagaacaggctaccttgcggagagtgtcctgcattccaaccgga

SEQ ID NO: 56 - DNA, 51 bp, synthetic construct
  source 1..51: mol_type = other DNA
gagagccaagaacaggctaccttgcggagagtgtcctgcattccaaccgga

SEQ ID NO: 57 - DNA, 37 bp, synthetic construct
  source 1..37: mol_type = other DNA
ggctaccttgcggagagtgtcctgcattccaaccgga

SEQ ID NO: 58 - DNA, 30 bp, synthetic construct
  source 1..30: mol_type = other DNA
ttgcggagagtgtcctgcattccaaccgga

SEQ ID NO: 59 - DNA, 48 bp, synthetic construct
  source 1..48: mol_type = other DNA
ccattaaaccatcccgaaggcgcgtaactgtactgagcgcgtcagcgc

SEQ ID NO: 60 - DNA, 33 bp, synthetic construct
  source 1..33: mol_type = other DNA
ccattaaaccatcccgaaggcgcgtaactgtac

SEQ ID NO: 61 - DNA, 18 bp, synthetic construct
  source 1..18: mol_type = other DNA
ccattaaaccatgaattc

SEQ ID NO: 62-63 - skipped (2 entries)

SEQ ID NO: 64 - DNA, 76 bp, synthetic construct
  source 1..76: mol_type = other DNA
ccatcttacctgggggcacggtagcctcaccggctctcccctcctaggcactacggccggggtgggtaaacggcgg

SEQ ID NO: 65 - DNA, 68 bp, synthetic construct
  source 1..68: mol_type = other DNA
ccatcttacctgggggcacggtagcctcaccggctctcccctcctaggcactacggccggggtgggta

SEQ ID NO: 66 - DNA, 38 bp, synthetic construct
  source 1..38: mol_type = other DNA
ccatcttacctgggggcacggtagcctcaccggctctc

SEQ ID NO: 67 - DNA, 26 bp, synthetic construct
  source 1..26: mol_type = other DNA
ccatcttacctgggggcacggtagcc

SEQ ID NO: 68 - DNA, 19 bp, synthetic construct
  source 1..19: mol_type = other DNA
ccatcttacctgggggcac

SEQ ID NO: 69 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 70 - protein, 1796 aa, synthetic construct
MDYKDHDGDYKDHDIDYKDDDDKPAAKRVKLDGGKRTADGSEFESMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKK

SEQ ID NO: 71 - protein, 1781 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKKPKKKRKV

SEQ ID NO: 72 - protein, 1803 aa, synthetic construct
MDYKDHDGDYKDHDIDYKDDDDKPAAKRVKLDGGKRTADGSEFESMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKKPKKKRKV

SEQ ID NO: 73 - protein, 1808 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKK

SEQ ID NO: 74 - protein, 1837 aa, synthetic construct
MDYKDHDGDYKDHDIDYKDDDDKPAAKRVKLDGGKRTADGSEFESMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKKPKKKRKV

SEQ ID NO: 75 - DNA, 42 bp, synthetic construct
  source 1..42: mol_type = other DNA
ttgacgcggttctatctagttacgcgttaaaccaactagaaa

SEQ ID NO: 76 - DNA, 37 bp, synthetic construct
  source 1..37: mol_type = other DNA
cgcggttctatctagttacgcgttaaaccaactagaa

SEQ ID NO: 77 - DNA, 56 bp, synthetic construct
  source 1..56: mol_type = other DNA
gggtcaggagccccccccctgaacccaggataaccctcaaagtcggggggcaaccc

SEQ ID NO: 78 - DNA, 78 bp, synthetic construct
  source 1..78: mol_type = other DNA
tagtcaggccagccggttaggctgccaccgaaggttggtagacggtgctgcctgcgaccaaccccaggaggactgggt

SEQ ID NO: 79 - DNA, 77 bp, synthetic construct
  source 1..77: mol_type = other DNA
agtcaggccagattaatgctgccaccggaagttgagtagacggtgctgcctgcggctcaaccccaggaggactgggt

SEQ ID NO: 80 - DNA, 81 bp, synthetic construct
  source 1..81: mol_type = other DNA
tgtcaggcctgctagtcagccacagtttggggaaagctgtgcagcctgtaacccccccaggagaagctgggaaaccaagct

SEQ ID NO: 81 - DNA, 69 bp, synthetic construct
  source 1..69: mol_type = other DNA
agtcaggccacttgtgccacggtttgagcaaaccgtgctgcctgtagctccgccaataatgggaggcgt

SEQ ID NO: 82 - DNA, 94 bp, synthetic construct
  source 1..94: mol_type = other DNA
tgtcagcccagaaccccacacgagttttgccactgctaagctgtgaggcagtgcaggctgggacagccgacctccaggttgcgaaaaacctggt

SEQ ID NO: 83 - protein, 230 aa, synthetic construct
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDG

SEQ ID NO: 84 - DNA, 692 bp, synthetic construct
  source 1..692: mol_type = other DNA
atggccaaaaagatcttcacatccgctctgggcacagccgagccttacgcctacatcgccaagccagactacggcaacgaggaacggggcttcggaaatcccagaggtgtgtacaaggtggacctgaccatccccaacaaggaccccagatgtcagagaatggtggatgaaatcgtgaagtgccacgaggaggcctacgccgctgctgttgaagagtacgaggctaatcctccagccgtggccagaggcaaaaaacctctgaaaccttacgagggcgatatgcctttcttcgacaacggcgacggcaccaccacctttaagttcaagtgctacgccagttttcaggacaagaagaccaaggaaacaaagcacatcaacctggtcgtggtggacagcaagggcaagaagatggaagatgtccccattatcggcggcggctctaaactgaaggtgaaatacagcctggtgccttataagtggaacaccgccgtgggcgccagcgtgaagctccagctggaatccgtgatgctggtggagctggccacattcggcggcggcgaggacgactgggccgacgaggtggaagaaaacggctacgtggccagcggcagcgccaaggcctctaagcctcgggacgaagagagctgggacgaggacgatgaggaaagcgaggaagctgatgaggatggaga

SEQ ID NO: 85 - protein, 269 aa, synthetic construct
MTKQPPIAKADLQKTQGNRAPAAIKNNDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFNFEFGLDEKLIHRPGENEDAPVTHVYAVARLKDGGTQFEVMTRKQIELVRSQSKAGNNGPWVTHWEEMAKKTAIRRLFKYLPVSIEIQRAVSMDEKEPLTIDPADSSVLTGEYSVIDNSEE

SEQ ID NO: 86 - DNA, 807 bp, synthetic construct
  source 1..807: mol_type = other DNA
atgaccaagcagccccccatcgctaaggccgatctgcagaagacacaaggcaaccgggcccctgctgccatcaagaacaacgacgtgatcagcttcatcaaccagccttctatgaaagaacagctggccgccgctctgcccagacacatgacagccgagcggatgatcagaatcgccaccaccgagatcaggaaggtgccagccctgggcaattgcgacaccatgtcttttgtgtccgcaatcgtgcaatgtagccagctgggcctcgagcctggcagtgctcttggccacgcctatctgctgccttttggcaacaagaatgagaaaagcggaaagaagaatgtgcagctgatcatcggctacagaggaatgatcgacctggccagaagaagcggccagatcgcctctctgagcgctagagtggtgcgggaaggcgacgagttcaacttcgagttcggcctggatgaaaagctgatccacagacctggcgaaaacgaggacgcccctgtgacccacgtgtacgccgtggccagactgaaggacggcggaacccagttcgaggtcatgaccagaaaacagattgagctggtgcggtctcagtcaaaggccggcaacaacggcccttgggtcacacattgggaggaaatggccaagaaaaccgccatccggagactgttcaagtacctgcctgttagcatcgagatccagagagccgtgtccatggacgaaaaggaacccctgaccatcgatcccgccgacagctccgtgctgaccggcgagtacagcgtgattgataacagcgaggaa

SEQ ID NO: 87 - protein, 261 aa, synthetic construct
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 88 - DNA, 783 bp, synthetic construct
  source 1..783: mol_type = other DNA
atgtctacagccctggccaccctggctggcaagctggccgagagagtgggaatggacagcgtggacccccaggagctgatcaccaccctgcggcagaccgccttcaagggcgacgccagcgacgcccagtttatcgccctgctgatcgtcgctaatcaatacggcctgaacccctggaccaaggaaatctatgccttccccgacaaacagaacggcatcgtgcctgtggtgggcgttgacggctggtccagaatcatcaatgagaaccagcagttcgacggaatggatttcgagcaagataacgaaagctgtacatgcagaatctacagaaaggacagaaaccaccctatctgcgtgaccgagtggatggacgaatgtaggcgggaacctttcaaaaccagagagggcagagaaattaccggcccatggcagagccaccccaagcggatgctgagacacaaggccatgatccagtgcgccagactggcctttggcttcgctggcatctacgacaaggatgaggccgagcggatcgtggaaaacaccgcctacaccgctgaacggcaacctgagcgcgacatcacacctgtgaacgacgagacaatgcaggagattaacacactgctcatcgctctggacaagacctgggatgacgatctgctgcctctgtgcagccagatcttcagaagagatatccgggccagcagcgaactgacacaggccgaagccgtgaaggccctgggcttcctgaagcagaaagccgccgagcagaaggtggccgct

SEQ ID NO: 89 - protein, 33 aa, synthetic construct
SGGSSGGSSGSETPGTSESATPESSGGSSGGSS

SEQ ID NO: 90 - protein, 20 aa, synthetic construct
GGGGSGGGGSGGGGSGGGGS

SEQ ID NO: 91 - protein, 15 aa, synthetic construct
GGGGSGGGGSGGGGS

SEQ ID NO: 92 - protein, 5 aa, synthetic construct
GGGGS

SEQ ID NO: 93 - protein, 5 aa, synthetic construct
GGGGA

SEQ ID NO: 94 - protein, 7 aa, synthetic construct
PKKKRKV

SEQ ID NO: 95 - protein, 16 aa, synthetic construct
KRPAATKKAGQAKKKK

SEQ ID NO: 96 - protein, 9 aa, synthetic construct
PAAKRVKLD

SEQ ID NO: 97 - protein, 1752 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTKRPAATKKAGQAKKKK

SEQ ID NO: 98 - protein, 1839 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSGRPYVTLNLNGMFMDKFKPYSKSNAPITTLEKLSKALSISVEELKAIAELPLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHDLPINKRKTKIFHCSSEPIKVHGLRVDYDSPRLPSDEVKRIRASIHNLKLLAAKNNTKTSVAYRKEFNRCMGRVNKLGRVAHEKYESFKKQLQAIKPMPSKRDVAVIDAAIKSLELSYSKGNQNKHWYKRKYDLTRYKMIILTRSESFKEKLECFKSRLASLKPKRPAATKKAGQAKKKK

SEQ ID NO: 99 - protein, 1748 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSRIYSLIDSQTLMTKGFASEVMRSPEPPKKWDIAKKKGGMRTIYHPSSKVKLIQYWLMNNVFSKLPMHNAAYAFVKNRSIKSNALLHAESKNKYYVKIDLKDFFPSIKFTDFEYAFTRYRDRIEFTTEYDKELLQLIKTICFISDSTLPIGFPTSPLIANFVARELDEKLTQKLNAIDKLNATYTRYADDIIVSTNMKGASKLILDCFKRTMKEIGPDFKINIKKFKICSASGGSIVVTGLKVCHDFHITLHRSMKDKIRLHLSLLSKGILKDEDHNKLSGYIAYAKDIDPHFYTKLNRKYFQEIKWIQNLHNKVEKRPAATKKAGQAKKKK

SEQ ID NO: 100 - protein, 1751 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAWKRPAATKKAGQAKKKK

SEQ ID NO: 101 - protein, 1912 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTAKLESHVPAAPPVSAEAPAPTRPDAAKQEARRAHHEALRLRWKAIEEAGGTDAWVRQQLVAKGVAAEEVDFESLSDKQKAAWKEKKKAEATERRAQKRLAWEAWKATHIHHLGVGVHWDEAGGPDKFDVAGREERAKANGLPEGLDSVEALAKALGISVSRLRWFSFHREVDTGTHYQTWEIPKRDGGKRTLTAPKRELKAVQRWVLANVVERLPVHGAAHGFVAGRSILTNALAHQGADVVVKVDMKDFFPSVTWPRVKGLLRKGGLPENLATLLALLSTEAPREVVRFRGETLYVAKGPRALPQGAPTSPALTNALCLRLDKRLSALSKRLGFTYTRYADDLTFSWRRAKKSRQKELPLADAPVALLLARVKGVLEAEGFTLHPDKTRVQRKGSRQRVTGLVVNEAPEGVPGARVPRDVVRRLRAAIHNREQGKPGPTGETLEQLKGLAAFLHMTDAEKGRAFLRRLEALEKRQTAKRPAATKKAGQAKKKK

(19) United States
(12) Patent Application Publication
(10) Pub. No.: US 2026/0091137 A1
(43) Pub. Date: Apr. 2, 2026

(54) RETRON DIRECTED GENE EDITING

(71) Applicant: ENCELADUS BIO, INC., New York, NY (US)

(72) Inventors: Brian Busser, New York, NY (US); Johann Neil Reshan Cooke, New York, NY (US); Robin L. Armstrong, New York, NY (US); Gregory Mazo, New York, NY (US); Glenn Christopher Tan, New York, NY (US); Madyson Scherr, New York, NY (US)

(21) Appl. No.: 19/111,662

(22) PCT Filed: Sep. 15, 2023
(86) PCT No.: PCT/US2023/074333

§ 371 Date: Mar. 13, 2025

Related U.S. Application Data
(60) Provisional application No. 63/375,793, filed on Sep. 15, 2022.

(51) Int. Cl. A61K 48/00; C12N 9/12; C12N 9/22; C12N 15/11; C12N 15/85

(52) U.S. Cl. CPC A61K 48/0058; C12N 9/1276; C12N 9/226; C12N 15/11; C12N 15/85; C12N 2310/20; C12N 2800/107; C12N 2800/22

(57) ABSTRACT

Compounds, polypeptides, compositions, and nucleic acid molecules are provided herein that can be used, for example, to edit the genome of a cell, such as a human cell.

### CROSS-REFERENCE TO RELATED APPLICATIONS

This application claims priority to U.S. Provisional Application No. 63/375,793, filed Sep. 15, 2022, which is hereby incorporated by reference in its entirety.

### REFERENCE TO SEQUENCE LISTING SUBMITTED ELECTRONICALLY

The instant application contains a Sequence Listing which has been submitted electronically in XML filed format and is hereby incorporated by reference in its entirety. Said XML copy, created on Sep. 14, 2023, is named “FIS001WO.XML” and is 111,131 bytes in size.

### BACKGROUND

Genome editing with engineered nucleases has made editing genomic sequences possible. Without being bound to any particular theory, for example, engineered nucleases can be used to generate site-specific double-strand breaks (DSBs) followed by resolution of DSBs by endogenous cellular repair mechanisms. The outcome can be either mutation of a specific site through mutagenic nonhomologous end-joining, creating insertions or deletions at the site of the break, or precise change of a genomic sequence through homologous recombination using an exogenously introduced donor template. One example of this, is the use of the CRISPR/Cas system.

Genome editing remains inefficient, and, therefore, there is a need for compositions and methods to facilitate more efficient genome editing. The present embodiments satisfies these needs as well as others.

### BRIEF SUMMARY

In some embodiments, provided herein are methods of modifying, or inducing one or more sequence modifications in one or more, target nucleic acids of interest at one or more target loci within a genome of a host cell, such as a mammalian cell, comprising: (a) transforming the host cell with one or more vectors encoding a heterologous nucleic acid molecule and a guide RNA (gRNA), wherein the heterologous nucleic acid molecule comprises a first inverted repeat nucleic acid molecule sequence upstream of a coding region and a second inverted repeat nucleic acid molecule sequence downstream of the coding region, wherein the coding region comprises a nucleic acid molecule comprising: (i) an msr locus and an msd locus comprising the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2; and (ii) a donor DNA sequence within the msd locus, wherein the msr locus and the msd locus form a RT binding region; and (b) culturing the host cell or transformed progeny of the host cell under conditions sufficient for expressing from the one or more vectors the heterologous nucleic acid molecule comprising a retron transcript and a gRNA molecule to induce one or more sequence modifications in one or more target nucleic acids of interest at the one or more target loci within the genome.

In some embodiments, a retron-guide RNA (gRNA) cassette is provided, comprising: a retron comprising: a first inverted repeat sequence coding region; an msr locus and an msd locus; a donor DNA sequence within the msd locus; and a second inverted repeat sequence coding region; and optionally a gRNA coding region, wherein the msr locus and the msd locus comprise the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2.

In some embodiments, a polypeptide having the formula of: of (N1)q-C1-L1-(N2)qq-R1-(N3)qqq is provided, wherein C1 is a nuclease, L1 is a peptide linker, R1 is a RT protein, and N1 and N2 are each independently, a NLS sequence, and wherein q, qq, or qqq, are each independently, 0, 1, 2, or 3.

In some embodiments, a polypeptide comprising a SSAP, RT, and a nuclease, is provided, wherein: the C-terminus of the RT is linked to the N-terminus of the nuclease, and the C-terminus of the nuclease is linked to the N-terminus of the SSAP; or the N-terminus of the RT is linked to the C-terminus of the nuclease, and the N-terminus of the nuclease is linked to the C-terminus of the SSAP.

### BRIEF DESCRIPTION OF THE DRAWINGS

FIG. 1 illustrates non-limiting embodiments of a guide RNA linked to a retron RNA in 5′ or 3′ orientation.

FIG. 2 is a bar graph showing NHEJ and HDR efficiency for gene editing constructs in which the gRNA has been placed 5′ or 3′ to the retron.

FIG. 3 illustrates non-limiting embodiments of reverse transcriptase (RT) fusion proteins, where RT is fused or linked to a CAS9 nuclease (e.g., wild-type or nickase mutant) in various configurations and other RT fusion proteins comprising a CAS9 nuclease and a single-stranded annealing protein (SSAP).

FIG. 4 illustrates non-limiting embodiments of a retron RT fused to dCas9 (catalytically inactive) and SSAP; or SSAP linked directly to RT; or as separate proteins.

FIG. 5 is a bar graph showing NHEJ and HDR frequency during retron-mediated gene editing using nickase versions of Cas9, all in HEK293T cells. HEK293T BFP reporter cells. Cas9 nickases were Cas9H840A or Cas9D10A, or Cas9WT; retron RT was from Ec48 or Ec107; lengths of flanking homology are designated in base pairs (e.g., 50/50 or 50/75); and strands are designated as non-target (NTS) or target (TS). Cells were analyzed 7 days post-transfection on a flow cytometer.

FIG. 6 is a bar graph showing repair activity using dead (dCas9) or nickase (D10A or H840A) versions of Cas fused to a single-stranded annealing protein (SSAP; see FIG. 3).

FIG. 7A shows schematic of a retron-gRNA constructs with a structural motifs 3′ to the retron sequences. Structural motifs 5′ to the retron sequences are also feasible.

FIG. 7B is a plot of HDR activity for various retron constructs with and without structural motifs or specific knots.

FIG. 8A is a schematic diagram showing the secondary structure of the msd/msr non-protein-encoding RNA transcript of retron Ec107. The arrows show the insertion point of heterologous targeting sequences for editing, which involve no deletion of the msd/msr region (53S) or progressive deletion of the msd/msr region (36S, 12S, 2S and OS).

FIG. 8A-1 illustrates various embodiments comprising a donor DNA insert in a mutated msr/msd locus as provided for herein.

FIG. 8A-2 illustrates various embodiments comprising a donor DNA insert in a mutated msr/msd locus as provided for herein.

FIG. 8A-3 illustrates non-limiting embodiments of alignments of Coding Regions Comprising Ec107 msr and msd locus with DNA donor insert, shown as D1, inserted between the portions of the msd locus (double underlined portion).

FIG. 8A-4 illustrates non-limiting embodiments of alignments of Coding Regions Comprising Mx162 msr and msd locus with DNA donor insert, shown as D1, inserted between the portions of the msd locus (double underlined portion).

FIG. 8B is a bar graph showing Ec107 retron-mediated NHEJ and HDR efficiency in editing using the constructs described in FIG. 8A. The heterologous or targeting sequence are against the HEK3 locus in transfected HEK293T cells. Genomic DNA was harvested three days post-transfection and analyzed by next generation sequencing.

FIG. 9A is a schematic diagram showing the secondary structure of the msd/msr non-protein-encoding RNA transcript of retron Mx162. The arrows show the insertion point of heterologous targeting sequences for editing, which involve no deletion of the msd/msr region (56S) or progressive deletion of the msd/msr region (52S, 21S, 7S and OS).

FIG. 9B is a bar graph showing Mx162 retron-mediated NHEJ and HDR efficiency in editing using the constructs described in FIG. 9A. The heterologous or targeting sequence are against the HEK3 locus in transfected HEK293T cells. Genomic DNA was harvested three days post-transfection and analyzed by next generation sequencing.

FIG. 10 is a bar graph showing NHEJ and HDR efficiency for various Ec86 retron constructs targeting the HEK locus in transiently transfected HEK293T cells. The parenthetical in the labels for the constructs indicates the left and right homology arm length, so that (50/70) means a targeting sequence having 50-nt homology arm at the 5′ end and 70-nt homology arm at the 3′ end. Ec86 5′ or 3′ refers to the positioning of the gRNA relative to the retron ncRNA. ssODN refers to a single-stranded oligo nucleotide donor that was co-transfected with Cas9 and gRNA.

FIG. 11A are schematics of various fusion proteins containing SpCas9 and Ec107 RT separated by an XTEN peptide linker, with different numbers and types of nuclear localization signals (NLSs) distributed at different positions within the fusion protein. Although, a nuclease, such as CAS9 is illustrated, any other nuclease as provided for herein can be substituted for the nuclease and the RT can be replaced with other RT enzymes.

FIG. 11B is a plot of the NHEJ and HDR frequency for the various constructs shown in FIG. 11A. HEK293T cells were transiently transfected with 1) plasmid expressing Cas9 fused to the Ec86 retron RT with varying NLSs and 2) a plasmid expressing an Ec86 retron guide RNA targeting the HEK3 locus. Genomic DNA was harvested three days post-transfection and analyzed by next generation sequencing.

FIG. 12A-12B are bar graphs showing precise repair rates with RT-D10A and H840A-RT fusion conformations using the BFP/GFP conversion assay. 12A shows EC107 retron. 12B shows FC100 retron. (Assay details: transfection follow by flow analysis; Day 7 time point, precise repair=GFP+, indels=BFP−/GFP−).

FIG. 13A-13B are bar graphs showing precise repair rates with TS repair template with RT-D10A and H840a-RT at multiple loci (VEGFA, SERPINA1, FANCF, BFP/GFP conversion assay). 13A shows EC107-D10A and FC100-D10a. 13B shows H840a-EC107 and H840A-FC100. (Assay details: Transfection follow by MiSeq amplicon sequencing at VEGFA, SERPINA1, FANCF; Day 4 time point, CRISPResso analysis. Transfection follow by flow analysis; Day 7 time point, precise repair=GFP+).

FIG. 14A-14B are bar graphs showing precise repair rates with 4-component unfused nickase editing system than 2-component fused nickase editing system. 14A is EMX1. (Assay details: Transfection follow by MiSeq amplicon sequencing; Day 4 time point, CRISPResso analysis.) 14B is BFP/GFP conversion assay. (Assay details: Transfection follow by flow analysis at; Day 7 time point, precise repair=GFP+, indels=BFP−/GFP−).

### DETAILED DESCRIPTION

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±5% and remain within the scope of the disclosed embodiments. Thus, about 100 means 95 to 105.

As used herein, the term “animal” includes, but is not limited to, humans and non-human vertebrates such as wild, domestic, and farm animals. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused,” “linked,” or “conjugated” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequence, such as the glycine/serine sequences described herein to link the two domains together. Two peptide sequences are linked directly if they are directly connected to one another or indirectly if there is a linker or other structure that links the two regions. A linker can be directly linked to two different peptide sequences or domains.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As provided herein, the therapeutic compounds and compositions can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an auto-immune disease/disorder” means an activity that alleviates or ameliorates any of the primary phenomena or secondary symptoms associated with the auto-immune disease/disorder or other condition described herein. The various disease or conditions are provided herein. The therapeutic treatment can also be administered prophylactically to preventing or reduce the disease or condition before the onset.

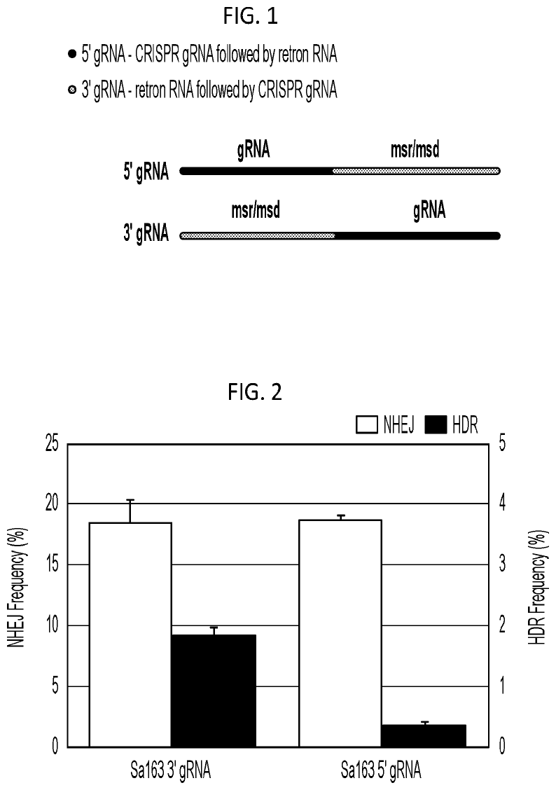

As used herein, unless otherwise specified, the terms “5” and “3” denote the positions of elements or features relative to the overall arrangement of polynucleotide sequence, such as a retron-guide RNA cassettes, vectors, or retron donor DNA-guide molecules, or other polynucleotide sequences encoding for a molecule of interest, such as a fusion protein provided for herein. Positions are not, unless otherwise specified, referred to in the context of the orientation of a particular element or features. For example, FIG. 1 illustrates the msr and msd sequence at oriented to the 5′ end of gRNA sequence or the 3′ end of the gRNA sequence. This is a non-limiting example and the gRNA can be part of a different transcript and expressed from a different vector. Unless otherwise specified, the term “upstream” refers to a position that is 5′ of a point of reference. Conversely, the term “downstream” refers to a position that is 3′ of a point of reference. Thus, in FIG. 1 the msr/msd sequence locus is said to be located downstream (top) or upstream (bottom) of the gRNA sequence.

The term “genome editing” refers to a type of genetic engineering in which DNA is inserted, replaced, or removed from a target DNA (e.g., the genome of a cell) using one or more nucleases and/or nickases. The nucleases can create specific double-strand breaks (DSBs), single strand breaks at desired locations in the genome, and use the cell's endogenous mechanisms to repair the induced break by homology-directed repair (HDR) (e.g., homologous recombination) or by nonhomologous end joining (NHEJ). The nickases create specific single-strand breaks at desired locations in the genome. In one non-limiting example, two nickases can be used to create two single-strand breaks on opposite strands of a target DNA, thereby generating a blunt or a sticky end. Any suitable DNA nuclease can be introduced into a cell to induce genome editing of a target DNA sequence. Genome editing can be performed with a catalytic inactive form of a Cas, which can be referred to as dCAS.

The term “DNA nuclease” refers to an enzyme capable of cleaving the phosphodiester bonds between the nucleotide subunits of DNA, and may be an endonuclease or an exonuclease. According to the present invention, the DNA nuclease may be an engineered (e.g., programmable or targetable) DNA nuclease which can be used to induce genome editing of a target DNA sequence. Any suitable DNA nuclease can be used including, but not limited to, CRISPR-associated protein (Cas) nucleases, other endo- or exo-nucleases, variants thereof, fragments thereof, and combinations thereof. In some embodiments, a DNA nuclease can be mutated to create a single strand break as opposed to a double stranded break. In some embodiments, the DNA nuclease is mutated to be catalytically inactive.

The term “double-strand break” or “double-strand cut” refers to the severing or cleavage of both strands of the DNA double helix. The DSB may result in cleavage of both stands at the same position leading to “blunt ends” or staggered cleavage resulting in a region of single-stranded DNA at the end of each DNA fragment, or “sticky ends”. A DSB may arise from the action of one or more DNA nucleases.

The term “nonhomologous end joining” or “NHEJ” refers to a pathway that repairs double-strand DNA breaks in which the break ends are directly ligated without the need for a homologous template.

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA. The repair can also be referred to as “recombineering,” which can utilize SSAP (as defined herein) to replace/fix a sequence during cell division.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “single nucleotide polymorphism” or “SNP” refers to a change of a single nucleotide within a polynucleotide, including within an allele. This can include the replacement of one nucleotide by another, as well as the deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position.

The term “gene” refers to a portion of DNA involved that encodes a polypeptide chain or other molecule, such as miRNA. The DNA may include regions preceding and following the coding region (leader and trailer) involved in the transcription/translation of the gene product and the regulation of the transcription/translation, as well as intervening sequences (introns) between individual coding segments (exons).

The term “cassette” refers to a heterologous combination of nucleic acid molecule elements that can be introduced as a single element and may function together to achieve a desired result.

The term “operably linked” refers to two or more genetic elements, such as a polynucleotide coding sequence and a promoter, placed in relative positions that permit the proper biological functioning of the elements, such as the promoter directing transcription of the coding sequence.

The term “inducible promoter” refers to a promoter that responds to environmental factors and/or external stimuli that can be artificially controlled in order to modify the expression of, or the level of expression of, a polynucleotide sequence or refers to a combination of elements, for example an exogenous promoter and an additional element such as a trans-activator operably linked to a separate promoter. An inducible promoter may respond to abiotic factors such as oxygen levels or to chemical or biological molecules. In some embodiments, the chemical or biological molecules may be molecules not naturally present in humans.

The terms “vector” and “expression vector” refer to a nucleic acid construct, generated recombinantly or synthetically, with a series of specified nucleic acid elements configured to transcribe a molecule of interest in a cell. An expression vector may be part of a plasmid, viral genome, or nucleic acid fragment. In some embodiments, the expression vector comprises a promoter operably linked to a heterologous polynucleotide sequence. In some embodiments, the vector is DNA, mRNA, or RNA. In some embodiments, the Cas-RT constructs or other constructs that encode for the RT or other proteins provided for herein can be delivered as a mRNA vector and the retron gRNA can be provided as RNA.

The term “promoter” is used herein to refer a nucleic acid sequences that directs, controls, or promotes the transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

“Recombinant” refers to a genetically modified polynucleotide, polypeptide, cell, tissue, or organism. A recombinant expression cassette, for example, can comprise a promoter operably linked to a second polynucleotide (e.g., a coding sequence) and can include a promoter that is heterologous to the second polynucleotide as the result of manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). A recombinant protein is one that is expressed from a recombinant polynucleotide, and recombinant cells, tissues, and organisms are those that comprise recombinant sequences (polynucleotide and/or polypeptide).

As used herein, the term “heterologous” refers to biological material that is introduced, inserted, or incorporated into a host (e.g., cell subject, tissue, etc.) that originates from another source. Heterologous material can include, but is not limited to, nucleic acids, amino acids, peptides, proteins, and structural elements such as genes, promoters, and cassettes. In some embodiments, a host cell is, but is not limited to, a bacterium, a yeast cell, a mammalian cell, or a plant cell. The introduction of heterologous material into a host cell or organism can result, in some instances, in the expression of additional heterologous material in or by the host cell or organism. As a non-limiting example, the transformation, transfection, or transduction of a mammalian host cell with an expression vector that contains DNA sequences encoding a bacterial protein (e.g. CAS9 or variants thereof) may result in the expression of the bacterial protein by the cell. The incorporation of heterologous material may be permanent or transient. Also, the expression of heterologous material may be permanent or transient.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “effective amount” or “sufficient amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the host cell type, the location of the host cell in the subject, the dosing regimen to be followed, whether it is administered in combination with other compounds, timing of administration, and the physical delivery system in which it is carried.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the patient. Non-limiting examples of pharmaceutically acceptable carrier include water, NaCl, normal saline solutions, lactated Ringer's, normal sucrose, normal glucose, cell culture media, and the like. One of skill in the art will recognize that other pharmaceutical carriers are useful in the present invention.

“Percent similarity,” in the context of polynucleotide or peptide sequences, is determined by comparing two optimally aligned sequences over a comparison window, wherein the portion of the sequence (e.g., an msr locus sequence) in the comparison window may comprise additions or deletions (i.e., gaps) as compared to the reference sequence which does not comprise additions or deletions, for optimal alignment of the two sequences. The percentage is calculated by determining the number of positions at which the identical nucleotide or amino acid occurs in both sequences to yield the number of matched positions, dividing the number of matched positions by the total number of positions in the window of comparison and multiplying the result by 100 to yield the percentage of similarity (e.g., sequence similarity).

When a polynucleotide or peptide has at least about 70% similarity (e.g., sequence similarity), preferably at least about 85%, at least about 86%, at least about 87%, at least about 88%, at least about 89%, at least about 90%, at least about 91%, at least about 92%, at least about 93, at least about 94%, at least about 95%, at least about 96%, at least about 97%, at least about 98%, at least about 99%, or at least about 100% similarity, to a reference sequence, when compared and aligned for maximum correspondence over a comparison window, or designated region as measured using one of the following sequence comparison algorithms or by manual alignment and visual inspection, such sequences are then said to be “substantially similar.” In some embodiments, a polynucleotide or peptide has about 70% similarity (e.g., sequence similarity), preferably about 85%, about 86%, about 87%, about 88%, about 89%, about 90%, about 91%, about 92%, about 93, about 94%, about 95%, about 96%, about 97%, about 98%, about 99%, or about 100% similarity, to a reference sequence, when compared and aligned for maximum correspondence over a comparison window, or designated region as measured using one of the following sequence comparison algorithms or by manual alignment and visual inspection, such sequences are then said to be “substantially similar.” In some embodiments, a polynucleotide or peptide has at least 70% similarity (e.g., sequence similarity), preferably at least 85%, at least 86%, at least 87%, at least 88%, at least 89%, at least 90%, at least 91%, at least 92%, at least 93, at least 94%, at least 95%, at least 96%, at least 97%, at least 98%, at least 99%, or 100% similarity, to a reference sequence, when compared and aligned for maximum correspondence over a comparison window, or designated region as measured using one of the following sequence comparison algorithms or by manual alignment and visual inspection, such sequences are then said to be “substantially similar.” With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence similarities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Additional examples of algorithms that are suitable for determining percent sequence similarity are the BLAST and BLAST 2.0 algorithms, which are described in Altschul et al., (1990) J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

Retrons. Retrons have been known for some time as a class of retroelement, first discovered in gram-negative bacteria such as Myxococcus xanthus (e.g., retrons Mx65 and Mx162), Stigmatella aurantiaca (e.g., retron Sa163), and Escherichia coli (e.g., retrons Ec48, Ec67, Ec73, Ec78, Ec83, Ec86, and Ec107). Retrons are also found in Salmonella typhimurium (e.g., retron St85), Salmonella enteritidis, Vibrio cholera (e.g., retron Vc95), Vibrio parahaemolyticus (e.g., retron Vp96), Klebsiella pneumoniae, Proteus mirabilis, Xanthomonas campestris, Rhizobium sp., Bradyrhizobium sp., Ralstonia metallidurans, Nannocystis exedens (e.g., retron Ne144), Geobacter sulfurreducens, Trichodesmium erythraeum, Nostoc punctiforme, Nostoc sp., Staphylococcus aureus, Fusobacterium nucleatum, and Flexibacter elegans. In some embodiments a retron-guide RNA cassette is provided that comprise a retron. In some embodiments, the retron is derived from the E. coli retron Ec86, which is, for example, illustrated in FIG. 2 in U.S. Patent Application Publication No. 20190330619A1, which is hereby incorporated by reference in its entirety.

In some embodiments, the retron comprises a non-coding RNA (ncRNA). In some embodiments, the ncRNA comprises a msr/msd locus. In some embodiments, an msr/msd locus comprises an msr locus and a msd locus. In some embodiments, a ncRNA comprises a first inverted repeat sequence coding region. In some embodiments, a ncRNA comprises a second inverted repeat sequence coding region. In some embodiments, a ncRNA comprises a donor DNA sequence. In some embodiments, a donor DNA sequence is located within an msr/msd locus. In some embodiments, a donor DNA sequence is located within an msd locus. In some embodiments, a retron comprises an msr/msd locus, a first inverted repeat sequence coding region, a donor DNA sequence, and a second inverted repeat sequence coding region. In some embodiments, the ncRNA comprises the msr/msd locus, the first inverted repeat sequence coding region, the donor DNA sequence, and the second inverted repeat sequence coding region. In some embodiments, the retron comprises the msr locus, the first inverted repeat sequence coding region, the msd locus, the donor DNA sequence, and the second inverted repeat sequence coding region. In some embodiments, the ncRNA comprises the msr locus, the first inverted repeat sequence coding region, the msd locus, the donor DNA sequence, and the second inverted repeat sequence coding region. In some embodiments, the retron comprises the first inverted repeat sequence coding region, the msr locus, the msd locus, the second inverted repeat sequence coding region, and the RT. In some embodiments, the retron comprises the first inverted repeat sequence coding region, the msr locus, the msd locus, the donor DNA sequence within the msd locus, the second inverted repeat sequence coding region, and the RT. In some embodiments, the retron comprises an ncRNA and an RT.

In some embodiments, the heterologous nucleic acid molecule comprises a non-coding RNA (ncRNA). In some embodiments, the ncRNA comprises a msr/msd locus. In some embodiments, an msr/msd locus comprises an msr locus and a msd locus. In some embodiments, a ncRNA comprises a first inverted repeat sequence coding region. In some embodiments, a ncRNA comprises a second inverted repeat sequence coding region. In some embodiments, a ncRNA comprises a donor DNA sequence. In some embodiments, a donor DNA sequence is located within an msr/msd locus. In some embodiments, a donor DNA sequence is located within an msd locus. In some embodiments, a heterologous nucleic acid molecule comprises an msr/msd locus, a first inverted repeat sequence coding region, a donor DNA sequence, and a second inverted repeat sequence coding region. In some embodiments, the ncRNA comprises the msr/msd locus, the first inverted repeat sequence coding region, the donor DNA sequence, and the second inverted repeat sequence coding region. In some embodiments, the heterologous nucleic acid molecule comprises the msr locus, the first inverted repeat sequence coding region, the msd locus, the donor DNA sequence, and the second inverted repeat sequence coding region. In some embodiments, the ncRNA comprises the msr locus, the first inverted repeat sequence coding region, the msd locus, the donor DNA sequence, and the second inverted repeat sequence coding region. In some embodiments, the heterologous nucleic acid molecule comprises the first inverted repeat sequence coding region, the msr locus, the msd locus, the second inverted repeat sequence coding region, and the RT. In some embodiments, the heterologous nucleic acid molecule comprises the first inverted repeat sequence coding region, the msr locus, the msd locus, the donor DNA sequence within the msd locus, the second inverted repeat sequence coding region, and the RT. In some embodiments, the heterologous nucleic acid molecule comprises an ncRNA and an RT.

Examples of RT's that can be used, include, but are not limited to:

KSAEYLNTFRLRNLGLPVMNNLHDMSKATRISVETLRLLIYTADFRYRIY
TVEKKGPEKRMRTIYQPSRELKALQGWVLRNILDKLSSSPFSIGFEKHQS
ILNNATPHIGANFILNIDLEDFFPSLTANKVFGVFHSLGYNRLISSVLTK
ICCYKNLLPQGAPSSPKLANLICSKLDYRIQGYAGSRGLIYTRYADDLTL
SAQSMKKVVKARDFLFSIIPSEGLVINSKKTCISGPRSQRKVTGLVISQE
KVGIGREKYKEIRAKIHHIFCGKSSEIEHVRGWLSFILSVDSKSHRRLIT
YISKLEKKYGKNPLNKAKT (Full Name: Retron-
Eco1; Abbrev Name: Ec86; Type: RT;
SEQ ID NO: 44);
RIYSLIDSQTLMTKGFASEVMRSPEPPKKWDIAKKKGGMRTIYHPSSKVK
LIQYWLMNNVFSKLPMHNAAYAFVKNRSIKSNALLHAESKNKYYVKIDLK
DFFPSIKFTDFEYAFTRYRDRIEFTTEYDKELLQLIKTICFISDSTLPIG
FPTSPLIANFVARELDEKLTQKLNAIDKLNATYTRYADDIIVSTNMKGAS
KLILDCFKRTMKEIGPDFKINIKKFKICSASGGSIVVTGLKVCHDFHITL
HRSMKDKIRLHLSLLSKGILKDEDHNKLSGYIAYAKDIDPHFYTKLNRKY
FQEIKWIQNLHNKVE (Full Name: Retron-Eco3;
Abbrev Name: Ec73; Type: RT; SEQ ID
NO: 45);
GRPYVTLNLNGMFMDKFKPYSKSNAPITTLEKLSKALSISVEELKAIAEL
PLDEKYTLKEIPKIDGSKRIVYSLHPKMRLLQSRINKRIFKELVVFPSFL
FGSVPSKNDVLNSNVKRDYVSCAKAHCGAKTVLKVDISNFFDNIHRDLVR
SVFEEILHIKDEALEYLVDICTKDDFVVQGALTSSYIATLCLFAVEGDVV
RRAQRKGLVYTRLVDDITVSSKISNYDFSQMQSHIERMLSEHDLPINKRK
TKIFHCSSEPIKVHGLRVDYDSPRLPSDEVKRIRASIHNLKLLAAKNNTK
TSVAYRKEFNRCMGRVNKLGRVAHEKYESFKKQLQAIKPMPSKRDVAVID
AAIKSLELSYSKGNQNKHWYKRKYDLTRYKMIILTRSESFKEKLECFKSR
LASLKP (Full Name: Retron-Eco6; Abbrev
Name: Ec48; Type: RT; SEQ ID NO:
46);
DATRTTLLALDLFGSPGWSADKEIQRLHALSNHAGRHYRRIILSKRHGGQ
RLVLAPDYLLKTVQRNILKNVLSQFPLSPFATAYRPGCPIVSNAQPHCQQ
PQILKLDIENFFDSISWLQVWRVFRQAQLPRNVVTMLTWICCYNDALPQG
APTSPAISNLVMRRFDERIGEWCQARGITYTRYCDDMTFSGHFNARQVKN
KVCGLLAELGLSLNKRKGCLIAACKRQQVTGIVVNHKPQLAREARRALRQ
EVHLCQKYGVISHLSHRGELDPSGDLHAQATAYLYALQGRINWLLQINPE
DEAFQQARESVKRMLVAW (Full Name: Retron-
Eco5; Abbrev Name: Ec107; Type: RT;
SEQ ID NO: 47);
and
TAKLESHVPAAPPVSAEAPAPTRPDAAKQEARRAHHEALRLRWKAIEEAG
GTDAWVRQQLVAKGVAAEEVDFESLSDKQKAAWKEKKKAEATERRAQKRL
AWEAWKATHIHHLGVGVHWDEAGGPDKFDVAGREERAKANGLPEGLDSVE
ALAKALGISVSRLRWFSFHREVDTGTHYQTWEIPKRDGGKRTLTAPKREL
KAVQRWVLANVVERLPVHGAAHGFVAGRSILTNALAHQGADVVVKVDMKD
FFPSVTWPRVKGLLRKGGLPENLATLLALLSTEAPREVVRFRGETLYVAK
GPRALPQGAPTSPALTNALCLRLDKRLSALSKRLGFTYTRYADDLTFSWR
RAKKSRQKELPLADAPVALLLARVKGVLEAEGFTLHPDKTRVQRKGSRQR
VTGLVVNEAPEGVPGARVPRDVVRRLRAAIHNREQGKPGPTGETLEQLKG
LAAFLHMTDAEKGRAFLRRLEALEKRQTA (Full Name:
Retron-Saul; Abbrev Name: Sal63; Type:
RT; SEQ ID NO: 48).

Without being bound by an particular theory, retrons mediate the synthesis in host cells of multicopy single-stranded DNA (msDNA) molecules, which result from the reverse transcription of a retron transcript and typically include a DNA component and an RNA component. The native msDNA molecules exist as single-stranded DNA-RNA hybrids, characterized by a structure which comprises a single-stranded DNA branching out of an internal guanosine residue of a single-stranded RNA molecule at a 2′,5′-phosphodiester linkage. In some embodiments of the present invention, at least some of the RNA content of the msDNA molecule is degraded. In some instances, the RNA content is degraded by RNase H.

Retrons have been found to consist of the gene for RT and msr and msd loci under the control of a single promoter. In some embodiments, a vector comprising a retron-guide RNA cassette is provided. In some embodiments, the cassette does not comprise a sequence encoding for a RT. Thus, in some embodiments, methods are provided wherein the RT is encoded on a separate plasmid from the retron-guide RNA cassette. In some embodiments, the RT is encoded in a sequence that has been integrated into the host cell genome.

In some embodiments, the msd region of a retron transcript typically codes for the DNA component of msDNA, and the msr region of a retron transcript typically codes for the RNA component of msDNA. In some embodiments of the retrons, the msr and msd loci have overlapping ends, and may be oriented opposite one another with a promoter located upstream of the msr locus which transcribes through the msr and msd loci. Examples of msd locus sequences are set forth in SEQ ID NOS: 19 and 30 in U.S. Patent Application Publication No. 20190330619A1, which is hereby incorporated by reference in its entirety. However, the specific sequence can vary due to the a specific donor DNA sequence that is located within the msd locus.

In some embodiments, an msd and msr region comprises an msd locus which is expressed in trans to an msr locus. In some embodiments, an msd and msr region comprises an msr locus that is expressed in trans to the msd locus. In some embodiments, an msd and msr region comprises an msr locus and an msd locus that is expressed in tandem to another msr locus and msd locus. In some embodiments, the msr locus and the msd locus that is expressed in tandem to another msr locus and msd locus, is expressed in trans. Non-limiting examples of msd and msr regions comprising an msd locus which is expressed in trans to an msr locus may be found in González-Delgado A, Lopez SC, Rojas-Montero M, Fishman CB, Shipman SL. Simultaneous multi-site editing of individual genomes using retron arrays. bioRxiv [Preprint]. 2023 Jul. 17:2023.07.17.549397. doi: 10.1101/2023.07.17.549397. PMID: 37503029; PMCID: PMC10370050, which is hereby incorporated by reference in its entirety.

A non-limiting example of an msr locus sequence is set forth in sequence identifier number 18 in U.S. Patent Application Publication No. 20190330619A1, which is hereby incorporated by reference in its entirety. In some embodiments, the msr locus within the retron of a retron-gRNA cassette of comprises the nucleotide sequence set forth in SEQ ID NO: 18 in U.S. Patent Application Publication No. 20190330619A1, which is hereby incorporated by reference in its entirety. In some embodiments, the msr locus comprises a nucleotide sequence that has at least 50% to about 99% similarity (e.g., at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similarity) to the nucleotide sequence set forth in sequence identifier eighteen (18) in U.S. Patent Application Publication No. 20190330619A1, which is hereby incorporated by reference in its entirety. The sequence of sequence identified as sequence 18 in the '619 publication is listed as:

(SEQ ID NO: 3)
atgcgcacccttagcgagaggtttatcattaaggtcaacctctggatgtt
gtttcggcatcctgcattgaatctgagttact.

In some embodiments, the msr and msd locus within the retron-gRNA cassette is as provided for herein, such as, but not limited to:

CGCCAGCAGTGGCAATAGCGTTTCCGGCCTTTTGTGCCGGGAGGGTCGGC
GAGTCGCTGACTTAACGCCAGTAGTATGTCCATATACCCAAAGTCGCTTC
ATTGTACCTGAGTACGCTTCGCGTGCGCTGACGCGCTCAGTACAGTTACG
CGCCTTCGGGATGGTTTAATGGTATTGCCGCTGTTGGCG (SEQ ID
NO: 1-Ec107 full length msr/msd locus);
or
GCGCGAGCAGCCGAGAGAGGTCCGGAGTGCATCAGCCTGAGCGCCTCGAG
CGGCGGAGCGGCGTTGCGCCGCTCCGGTTGGAATGCAGGACACTCTCCGC
AAGGTAGCCTGTTCTTGGCTCTCTCCCTCCTAGGCACTACGGCCAGGGTG
GGTAGCGGCCGCCGTTTACCCACCCCGGCCGTAGTGCCTAGGAGGGGAGA
GCCGGTGAGGCTACCGTGCCCCCAGGTAAGATGGTGGTGCTTTCCCGGCC
TCCCTCGACTGCTCGCGC (SEQ ID NO: 2--full length
Mx162 msr/msd locus).

In some embodiments, the msr/msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the msr/msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. In some embodiments, the msr/msd locus comprises the nucleic acid sequence of SEQ ID NO: 1. In some embodiments, the msr/msd locus comprises the nucleic acid sequence of SEQ ID NO: 2.

As provided for herein, the msr/msd locus can be modified (mutated) and still be used to initiate reverse transcription. Thus, in some embodiments, the mutated or modified msr/msd locus can be within the retron. In some embodiments, the retron comprises a mutated msd locus, such as provided for herein. In some embodiments, the retron comprises a mutated msr locus, such as provided for herein. In some embodiments, the retron comprises a mutated msd locus and msr locus that does not comprise any mutations or modifications.

In some embodiments, the msd and msr regions of retron transcripts contain first and second inverted repeat sequences, which can form a stable stem structure. Without being bound to any particular theory, the combined msr-msd region of the retron transcript serves not only as a template for reverse transcription but, by virtue of its secondary structure, also serves as a primer (i.e., self-priming) for msDNA synthesis by a RT. In some embodiments of retron-guide RNA cassettes, the first inverted repeat sequence coding region is located within the 5′ end of the msr locus. In other embodiments, the second inverted repeat sequence coding region is located 3′ of the msd locus. In some embodiments of retron donor DNA-guide molecules of the present invention, the first inverted repeat sequence is located within the 5′ end of the msr region. In other embodiments, the second inverted repeat sequence is located 3′ of the msd region. A non-limiting example is shown in FIG. 4 as illustrated in U.S. Patent Application Publication No. 20190330619A1, which is hereby incorporated by reference in its entirety, wherein the msr and msd loci are arranged in opposite orientations. The first inverted sequence repeat coding region is shown at the 5′ end of the cassette, while the second inverted sequence repeat coding region is shown near the 3′ end of the cassette.

Non-limiting sequences for inverted repeat sequence coding regions are set forth in SEQ ID NOS: sixteen and seventeen in U.S. Patent Application Publication No. 20190330619A1, which is hereby incorporated by reference in its entirety. In some embodiments, a retron found within a retron-gRNA cassette of the present invention contains an inverted repeat sequence coding region that comprises the nucleotide sequence set forth in SEQ ID NO: 16 or 17. As a non-limiting example, the retron can contain a first inverted repeat sequence coding region that comprises SEQ ID NO: 16 and a second inverted repeat sequence coding region that comprises SEQ ID NO: 17, or vice versa. In other embodiments, an inverted repeat sequence coding region comprises a nucleotide sequence that has at least about 50% to about 99% similarity (e.g., at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similarity) to the nucleotide sequence set forth in SEQ ID NO: 16 or 17 in U.S. Patent Application Publication No. 20190330619A1, which is hereby incorporated by reference in its entirety. As a non-limiting example, the retron can contain a first inverted repeat sequence coding region that has at least about 70 to about 99 percent similarity to SEQ ID NO: sixteen and a second inverted repeat sequence coding region that has at least about 70 to about 99 percent similarity to SEQ ID NO: seventeen, or vice versa. One of ordinary skill in the art will understand that the sequence of an inverted repeat sequence coding region can be varied, so long as the sequence of the counterpart inverted repeat sequence coding region within the same retron is also varied such that the two resulting inverted repeat sequences (i.e., present within a retron transcript) are complementary and allow for the formation of a stable stem structure.

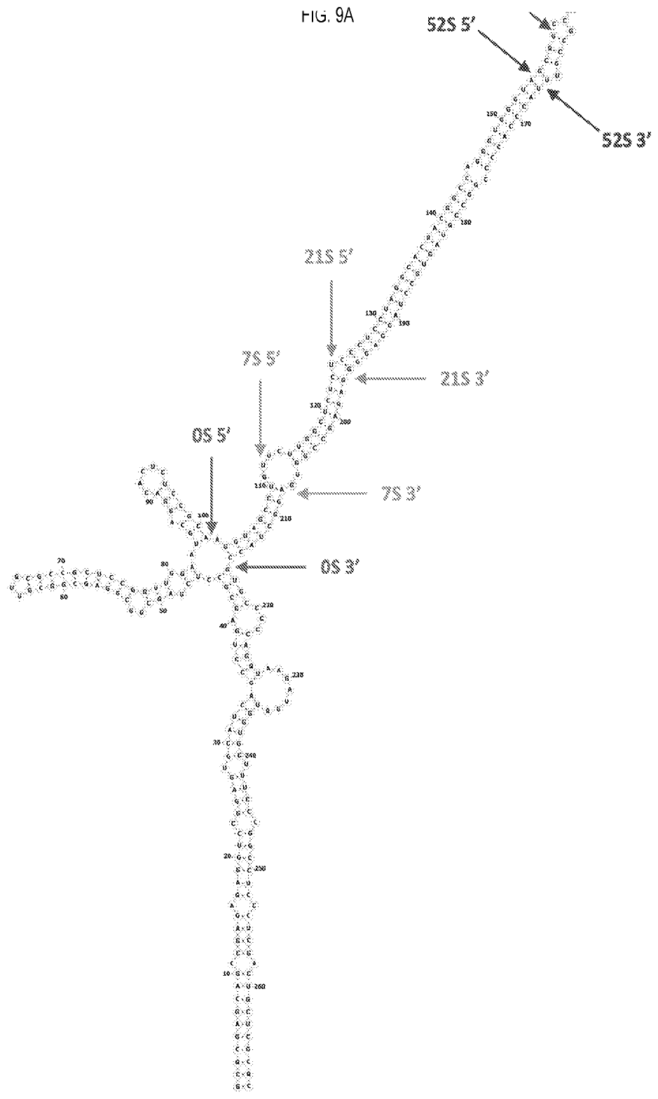

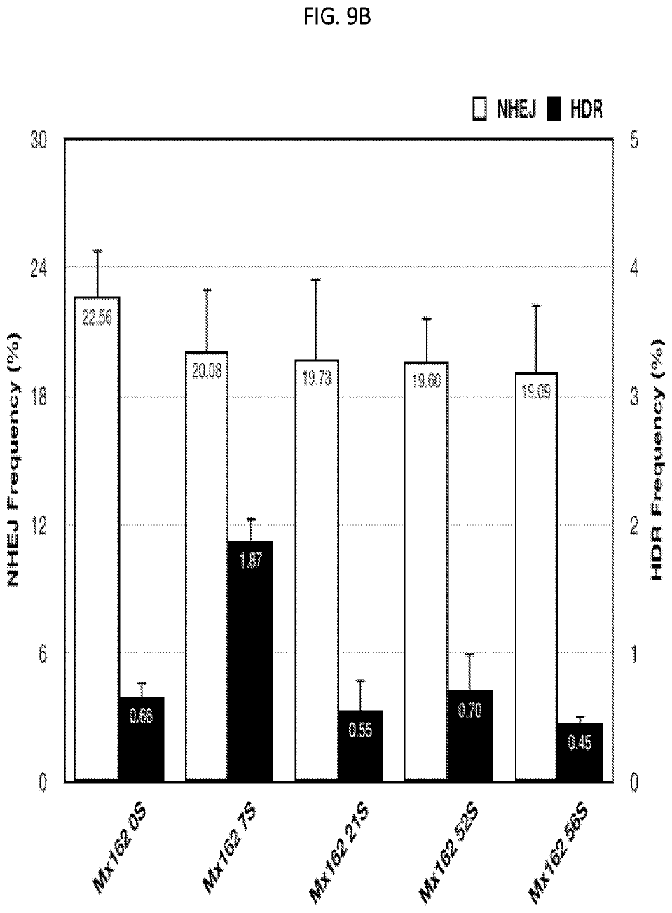

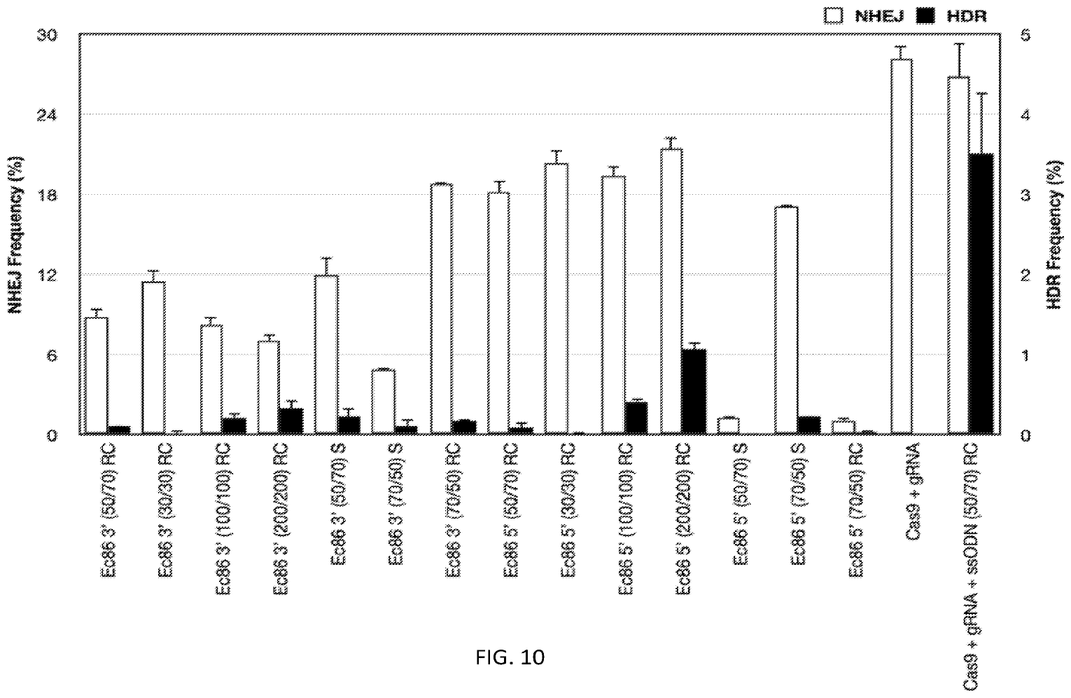

Without being bound to any particular theory, the stable stem structure can be used as a starting point for reverse transcription and the RT can initiate the transcription event by binding to this stem loop. For example, in some embodiments, RT can bind to the structure that is form between nucleotides of the msr locus. However, the embodiments, provided for herein have demonstrated that the sequence of the msr/msd locus can be modified (e.g, mutated, deleted, and insertions) such that the full-length msr/msd locus is not require to initiate reverse transcription. In contrast, a mutated msr/msd locus can be used to achieve greater efficiency in gene editing. In some embodiments, the msr locus sequence is not mutated or modified. In some embodiments, only the msd locus is mutated or modified as provided for herein. The mutated msr/msd locus can, in some embodiments, comprise the portion that binds to RT and where the reverse transcription can be initiated from. This portion of the locus is illustrated, for example, on FIG. 9A as 5′ end and the 3′ end interacting with one other. This region, which is formed by, as represented in the non-limiting embodiment illustrated in FIG. 9A, nucleotides 1-18, 1-19, or 1-20 and nucleotides 170-190, 171-190, and 172-190. What is illustrated in FIG. 9A is simply one example of locus that can be used and other msr/msd loci can be used to initiate RT as provided for herein.

Accordingly, is some embodiments, method of modifying, or inducing one or more sequence modifications in one or more, target nucleic acids of interest at one or more target loci within a genome of a host cell, such as a mammalian cell are provided. In some embodiments, the methods comprise:
(a) transforming the host cell with one or more vectors encoding a retron and a guide RNA (gRNA), which can be optionally, positioned to the 3′ or 5′ end of positioned of the retron when part of the same transcript, wherein the vector comprises a first inverted repeat nucleic acid molecule sequence upstream of a coding region and a second inverted repeat nucleic acid molecule sequence downstream of the coding region, wherein the coding region comprises a nucleic acid molecule comprising:
   (i) a msr locus, such as a mutated msr locus;
   (ii) a msd locus, such as a mutated msd locus;
   (iii) a donor DNA sequence,
wherein the donor DNA sequence is inserted in the msd locus; and
wherein the msr locus (including, if present, the mutated msr locus) and the msd locus (including, if present, the mutated msd locus) form a RT binding region; and
(b) culturing the host cell or transformed progeny of the host cell under conditions sufficient for expressing from the vector a retron donor DNA molecule comprising a retron transcript and a guide RNA (gRNA) molecule to induce one or more sequence modifications in one or more target nucleic acids of interest at the one or more target loci within the genome.

In some embodiments, the method comprises:
(a) transforming the host cell with one or more vectors encoding a retron and a guide RNA (gRNA), wherein the vector comprises a first inverted repeat nucleic acid molecule sequence upstream of a coding region and a second inverted repeat nucleic acid molecule sequence downstream of the coding region, wherein the coding region comprises a nucleic acid molecule comprising:
   (i) an msr locus and an msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2; and
   (ii) a donor DNA sequence within the msd locus,
   wherein the msr locus and the msd locus form a RT binding region; and
(b) culturing the host cell or transformed progeny of the host cell under conditions sufficient for expressing from the one or more vectors a retron donor DNA molecule comprising a retron transcript and a gRNA molecule to induce one or more sequence modifications in one or more target nucleic acids of interest at the one or more target loci within the genome.

In some embodiments, the method comprises:
(a) transforming the host cell with one or more vectors encoding a heterologous nucleic acid molecule and a guide RNA (gRNA), wherein the heterologous nucleic acid molecule comprises a first inverted repeat nucleic acid molecule sequence upstream of a coding region and a second inverted repeat nucleic acid molecule sequence downstream of the coding region, wherein the coding region comprises a nucleic acid molecule comprising:
(i) an msr locus and an msd locus comprising the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2; and
(ii) a donor DNA sequence within the msd locus,
wherein the msr locus and the msd locus form a RT binding region; and
(b) culturing the host cell or transformed progeny of the host cell under conditions sufficient for expressing from the one or more vectors the heterologous nucleic acid molecule comprising a retron transcript and a gRNA molecule to induce one or more sequence modifications in one or more target nucleic acids of interest at the one or more target loci within the genome.

In some embodiments, a retron-guide RNA (gRNA) cassette comprises:
a retron comprising:
   a first inverted repeat sequence coding region;
   an msr locus and an msd locus;
   a donor DNA sequence within the msd locus; and
   a second inverted repeat sequence coding region; and
optionally a gRNA coding region,
   wherein the msr locus and the msd locus comprise the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2.

In some embodiments, the msr and msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the msr and msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. In some embodiments, the msr and msd locus comprises the nucleic acid sequence of SEQ ID NO: 1. In some embodiments, the msr and msd locus comprises the nucleic acid sequence of SEQ ID NO: 2.

In some embodiments, the msr locus is a mutated msr locus and/or the msd locus is a mutated msd locus. In some embodiments, the mutated msr or msd locus comprises a deletion of at least 1 nucleotide as compared to the wild-type.

In some embodiments, the mutated msr locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to the wild-type. In some embodiments, the msr locus is not mutated. In some embodiments, the msr locus does not comprise a deletion in the first 10, 20, 30, 40, or 50 nucleotides from the 5′ end of the msr locus or the last 10, 20, 30, 40, or 50 nucleotides from the 3′ end of the msr locus.

In some embodiments, the mutated msd locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to the wild-type. In some embodiments, the msd locus comprises, consists, or consists essentially of the first 1, 2, 3, 4, 5, 10, 15, 20, 30, 1-30, 1-20, 1-10, 5-30, 5-20, 5-15, or 5-10 nucleotides, or any nucleotide number in the range (including the endpoints), of the 5′ end of the msd locus. In some embodiments, the msd locus comprises, consists, or consists essentially of the first 1, 2, 3, 4, 5, 10, 15, 20, 30, 1-30, 1-20, 1-10, 5-30, 5-20, 5-15, or 5-10 nucleotides, or any nucleotide number in the range (including the endpoints), of the 3′ end of the msd locus.

In some embodiments, the msr/msd locus comprises a mutation in the msd locus but does not comprise a mutation in the msr locus. The mutation in the msd locus can be as provided for herein.

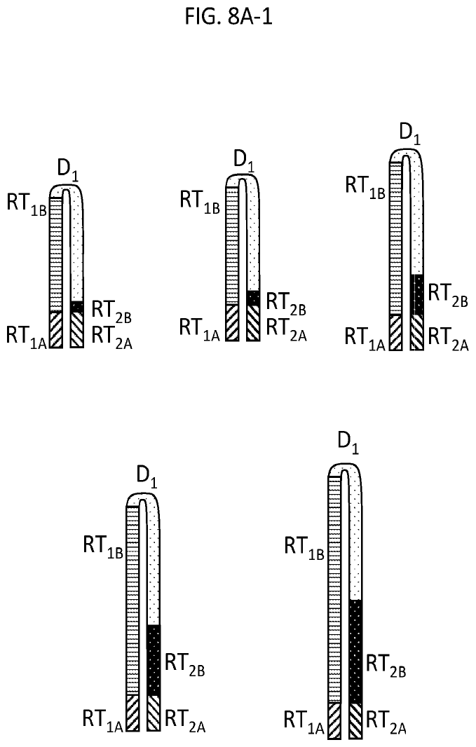

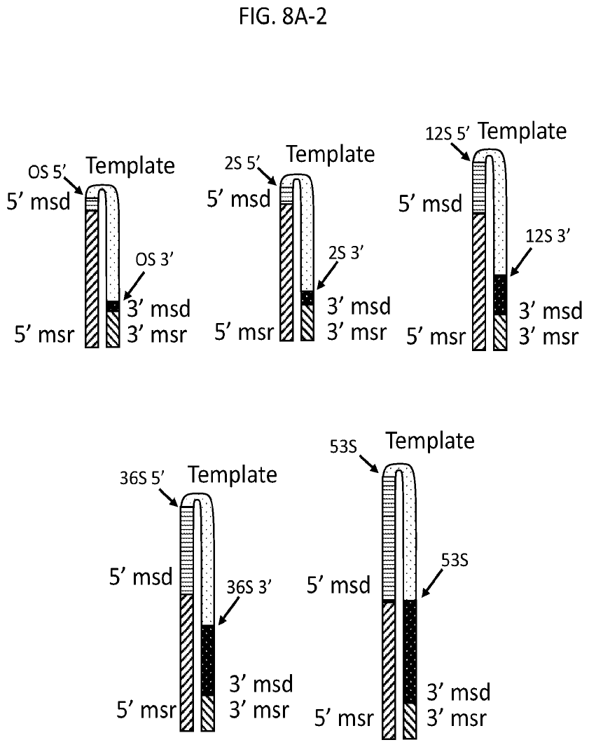

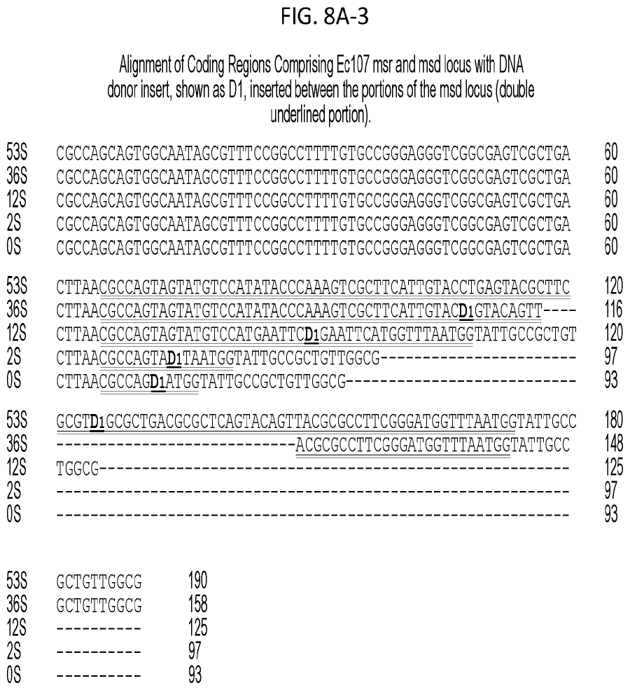

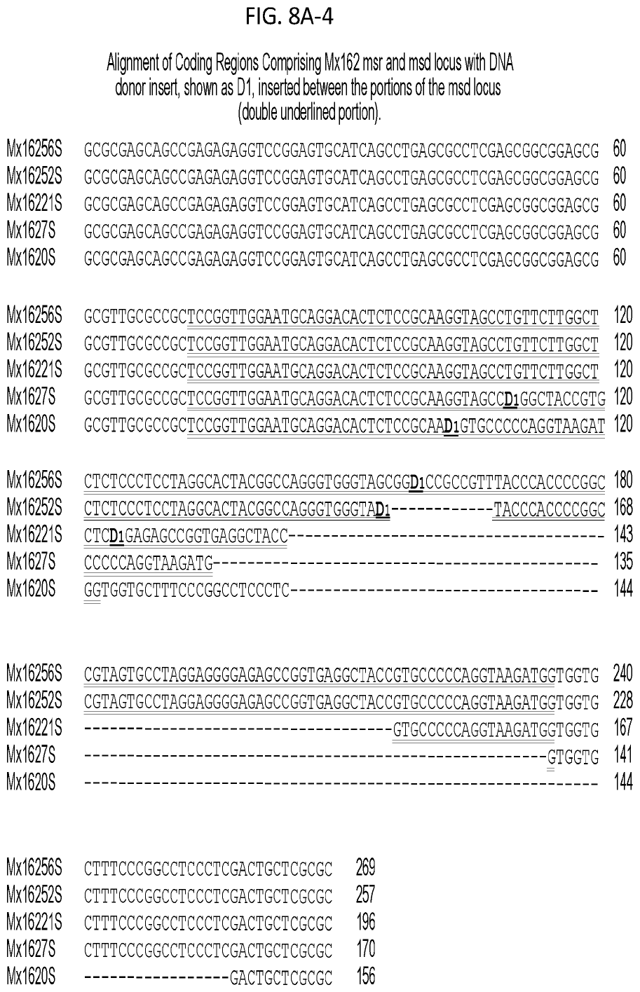

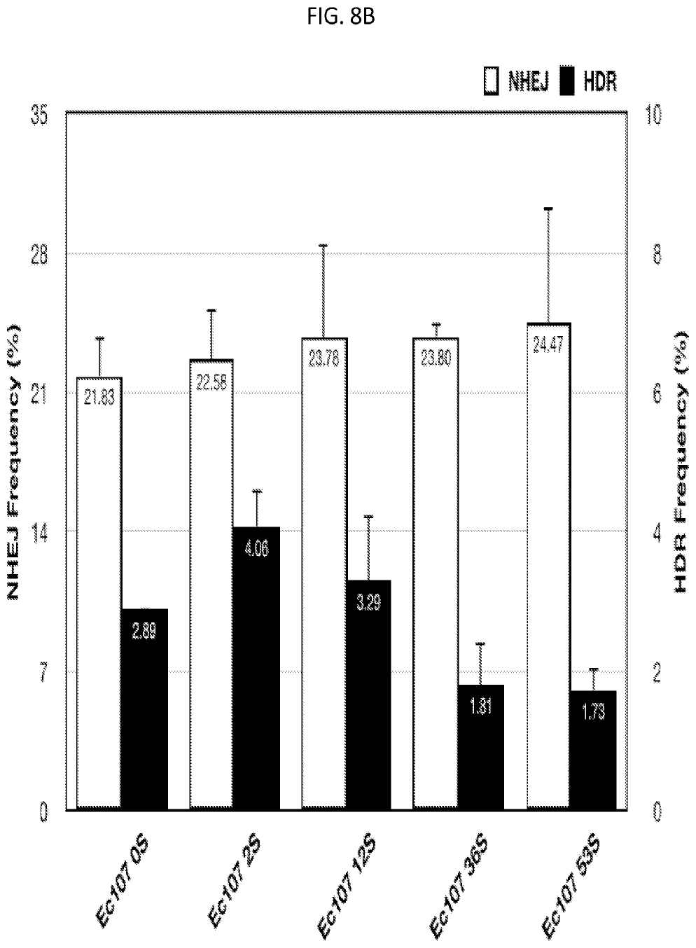

As provided for herein, the donor DNA sequence is inserted into the msr/msd locus and when the locus is reverse transcribed the donor DNA sequence is also reverse transcribed. The location of the insert can be anywhere downstream of the where the RT binds to. By downstream of where the RT binds to means that that the donor DNA insert is 3′ to the stem loop like structure that the RT binds to. For example, as illustrated in FIG. 8A, the donor DNA insert can be inserted at or after nucleotides, 18, 19, or 20. Conversely, the donor DNA insert is upstream of nucleotides 170, 171, or 172 as illustrated in FIG. 8A. The remaining portion of the msr/msd locus can be truncated by deletions or mutations to shorten the overall msr/msd locus, thereby allowing for the donor DNA insert to be, for example longer in length (more nucleotides).

In some embodiments, the msd locus upstream (to the 5′ of) of the donor DNA sequence comprises a nucleic acid sequence of:

(SEQ ID NO: 4)
CGCCAGTAGTATGTCCATATACCCAAAGTCGCTTCATTGTACCTGAGTAC
GCTTCGCGT;
(SEQ ID NO: 5)
CGCCAGTAGTATGTCCATATACCCAAAGTCGCTTCATTGTAC;
(SEQ ID NO: 6)
CGCCAGTAGTATGTCCATGAATTC;
(SEQ ID NO: 7)
CGCCAGTA;
(SEQ ID NO: 8)
CGCCAG;
(SEQ ID NO: 14)
TCCGGTTGGAATGCAGGACACTCTCCGCAAGGTAGCCTGTTCTTGGCTCT
CTCCCTCCTAGGCACTACGGCCAGGGGGGTAGCGG;
(SEQ ID NO: 15)
TCCGGTTGGAATGCAGGACACTCTCCGCAAGGTAGCCTGTTCTTGGCTCT
CTCCCTCCTAGGCACTACGGCCAGGGTGGGTA;
(SEQ ID NO: 16)
TCCGGTTGGAATGCAGGACACTCTCCGCAAGGTAGCCTGTTCTTGGCTCT
C;
(SEQ ID NO: 17)
TCCGGTTGGAATGCAGGACACTCTCCGCAAGGTAGCC;
or
(SEQ ID NO: 18)
TCCGGTTGGAATGCAGGACACTCTCCGCAA,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 4. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 5. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 6. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 7. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 8. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 14. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 15. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 16. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 17. In some embodiments, the msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 18. In some embodiments, the msd locus upstream of the donor DNA sequence comprises the nucleic acid sequence of SEQ ID NO: 4. In some embodiments, the msd locus upstream of the donor DNA sequence comprises the nucleic acid sequence of SEQ ID NO: 5. In some embodiments, the msd locus upstream of the donor DNA sequence comprises the nucleic acid sequence of SEQ ID NO: 6. In some embodiments, the msd locus upstream of the donor DNA sequence comprises the nucleic acid sequence of SEQ ID NO: 7. In some embodiments, the msd locus upstream of the donor DNA sequence comprises the nucleic acid sequence of SEQ ID NO: 8. In some embodiments, the msd locus upstream of the donor DNA sequence comprises the nucleic acid sequence of SEQ ID NO: 14. In some embodiments, the msd locus upstream of the donor DNA sequence comprises the nucleic acid sequence of SEQ ID NO: 15. In some embodiments, the msd locus upstream of the donor DNA sequence comprises the nucleic acid sequence of SEQ ID NO: 16. In some embodiments, the msd locus upstream of the donor DNA sequence comprises the nucleic acid sequence of SEQ ID NO: 17. In some embodiments, the msd locus upstream of the donor DNA sequence comprises the nucleic acid sequence of SEQ ID NO: 18.

In some embodiments, the mature retron comprises a msd reverse transcript upstream (to the 5′ of) of the donor DNA sequence comprises a nucleic acid sequence of:

(SEQ ID NO: 49)
ACGCGAAGCGTACTCAGGTACAATGAAGCGACTTTGGGTATATGGACATA
CTACTGGCG;
(SEQ ID NO: 50)
GTACAATGAAGCGACTTTGGGTATATGGACATACTACTGGCG;
(SEQ ID NO: 51)
GAATTCATGGACATACTACTGGCG;
(SEQ ID NO: 52)
TACTGGCG;
(SEQ ID NO: 53)
CTGGCG;
(SEQ ID NO: 54)
CCGCTACCCACCCTGGCCGTAGTGCCTAGGAGGGAGAGAGCCAAGAACAG
GCTACCTTGCGGAGAGTGTCCTGCATTCCAACCGGA;
(SEQ ID NO: 55)
TACCCACCCTGGCCGTAGTGCCTAGGAGGGAGAGAGCCAAGAACAGGCTA
CCTTGCGGAGAGTGTCCTGCATTCCAACCGGA;
(SEQ ID NO: 56)
GAGAGCCAAGAACAGGCTACCTTGCGGAGAGTGTCCTGCATTCCAACCGG
A;
(SEQ ID NO: 57)
GGCTACCTTGCGGAGAGTGTCCTGCATTCCAACCGGA;
or
(SEQ ID NO: 58)
TTGCGGAGAGTGTCCTGCATTCCAACCGGA,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 49. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 50. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 51. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 52. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 53. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 54. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 55. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 56. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 57. In some embodiments, the msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 58. In some embodiments, the msd reverse transcript upstream of the donor DNA sequence comprises the nucleic acid sequence of SEQ ID NO: 49. In some embodiments, the msd reverse transcript upstream of the donor DNA sequence comprises the nucleic acid sequence of SEQ ID NO: 50. In some embodiments, the msd reverse transcript upstream of the donor DNA sequence comprises the nucleic acid sequence of SEQ ID NO: 51. In some embodiments, the msd reverse transcript upstream of the donor DNA sequence comprises the nucleic acid sequence of SEQ ID NO: 52. In some embodiments, the msd reverse transcript upstream of the donor DNA sequence comprises the nucleic acid sequence of SEQ ID NO: 53. In some embodiments, the msd reverse transcript upstream of the donor DNA sequence comprises the nucleic acid sequence of SEQ ID NO: 54. In some embodiments, the msd reverse transcript upstream of the donor DNA sequence comprises the nucleic acid sequence of SEQ ID NO: 55. In some embodiments, the msd reverse transcript upstream of the donor DNA sequence comprises the nucleic acid sequence of SEQ ID NO: 56. In some embodiments, the msd reverse transcript upstream of the donor DNA sequence comprises the nucleic acid sequence of SEQ ID NO: 57. In some embodiments, the msd reverse transcript upstream of the donor DNA sequence comprises the nucleic acid sequence of SEQ ID NO: 58.

In some embodiments, the msd locus downstream (to the 3′ of) of the donor DNA sequence comprises a nucleic acid sequence of:

(SEQ ID NO: 9)
GCGCTGACGCGCTCAGTACAGTTACGCGCCTTCGGGATGGTTTAATGG;
(SEQ ID NO: 10)
GTACAGTTACGCGCCTTCGGGATGGTTTAATGG;
(SEQ ID NO: 11)
GAATTCATGGTTTAATGG;
(SEQ ID NO: 12)
TAATGG;
(SEQ ID NO: 13)
ATGG;
(SEQ ID NO: 19)
CCGCCGTTTACCCACCCCGGCCGTAGTGCCTAGGAGGGGAGAGCCGGTGA
GGCTACCGTGCCCCCAGGTAAGATGG;
(SEQ ID NO: 20)
TACCCACCCCGGCCGTAGTGCCTAGGAGGGGAGAGCCGGTGAGGCTACCG
TGCCCCCAGGTAAGATGG;
(SEQ ID NO: 21)
GAGAGCCGGTGAGGCTACCGTGCCCCCAGGTAAGATGG;
(SEQ ID NO: 22)
GGCTACCGTGCCCCCAGGTAAGATGG;
or
(SEQ ID NO: 23)
GTGCCCCCAGGTAAGATGG,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 9. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 11. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 12. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 13. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 19. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 21. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 22. In some embodiments, the msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the msd locus downstream of the donor DNA sequence comprises the nucleic acid sequence of SEQ ID NO: 9. In some embodiments, the msd locus downstream of the donor DNA sequence comprises the nucleic acid sequence of SEQ ID NO: 10. In some embodiments, the msd locus downstream of the donor DNA sequence comprises the nucleic acid sequence of SEQ ID NO: 11. In some embodiments, the msd locus downstream of the donor DNA sequence comprises the nucleic acid sequence of SEQ ID NO: 12. In some embodiments, the msd locus downstream of the donor DNA sequence comprises the nucleic acid sequence of SEQ ID NO: 13. In some embodiments, the msd locus downstream of the donor DNA sequence comprises the nucleic acid sequence of SEQ ID NO: 19. In some embodiments, the msd locus downstream of the donor DNA sequence comprises the nucleic acid sequence of SEQ ID NO: 20. In some embodiments, the msd locus downstream of the donor DNA sequence comprises the nucleic acid sequence of SEQ ID NO: 21. In some embodiments, the msd locus downstream of the donor DNA sequence comprises the nucleic acid sequence of SEQ ID NO: 22. In some embodiments, the msd locus downstream of the donor DNA sequence comprises the nucleic acid sequence of SEQ ID NO: 23.

In some embodiments, the mature retron comprises a msd reverse transcript downstream (to the 3′ of) of the donor DNA sequence comprises a nucleic acid sequence of:

(SEQ ID NO: 59)
CCATTAAACCATCCCGAAGGCGCGTAACTGTACTGAGCGCGTCAGCGC;
(SEQ ID NO: 60)
CCATTAAACCATCCCGAAGGCGCGTAACTGTAC;
(SEQ ID NO: 61)
CCATTAAACCATGAATTC;
(SEQ ID NO: 62)
CCATTA;
(SEQ ID NO: 63)
CCAT;
(SEQ ID NO: 64)
CCATCTTACCTGGGGGCACGGTAGCCTCACCGGCTCTCCCCTCCTAGGCA
CTACGGCCGGGGTGGGTAAACGGCGG;
(SEQ ID NO: 65)
CCATCTTACCTGGGGGCACGGTAGCCTCACCGGCTCTCCCCTCCTAGGCA
CTACGGCCGGGGTGGGTA;
(SEQ ID NO: 66)
CCATCTTACCTGGGGGCACGGTAGCCTCACCGGCTCTC;
(SEQ ID NO: 67)
CCATCTTACCTGGGGGCACGGTAGCC;
or
(SEQ ID NO: 68)
CCATCTTACCTGGGGGCAC,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 59. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 60. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 61. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 62. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 63. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 64. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 65. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 66. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 67. In some embodiments, the msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 68. In some embodiments, the msd reverse transcript downstream of the donor DNA sequence comprises the nucleic acid sequence of SEQ ID NO: 59. In some embodiments, the msd reverse transcript downstream of the donor DNA sequence comprises the nucleic acid sequence of SEQ ID NO: 60. In some embodiments, the msd reverse transcript downstream of the donor DNA sequence comprises the nucleic acid sequence of SEQ ID NO: 61. In some embodiments, the msd reverse transcript downstream of the donor DNA sequence comprises the nucleic acid sequence of SEQ ID NO: 62. In some embodiments, the msd reverse transcript downstream of the donor DNA sequence comprises the nucleic acid sequence of SEQ ID NO: 63. In some embodiments, the msd reverse transcript downstream of the donor DNA sequence comprises the nucleic acid sequence of SEQ ID NO: 64. In some embodiments, the msd reverse transcript downstream of the donor DNA sequence comprises the nucleic acid sequence of SEQ ID NO: 65. In some embodiments, the msd reverse transcript downstream of the donor DNA sequence comprises the nucleic acid sequence of SEQ ID NO: 66. In some embodiments, the msd reverse transcript downstream of the donor DNA sequence comprises the nucleic acid sequence of SEQ ID NO: 67. In some embodiments, the msd reverse transcript downstream of the donor DNA sequence comprises the nucleic acid sequence of SEQ ID NO: 68.

In some embodiments, the mutated msd locus upstream (to the 5′ of) of the donor DNA sequence comprises a nucleic acid sequence of:

(SEQ ID NO: 4)
CGCCAGTAGTATGTCCATATACCCAAAGTCGCTTCATTGTACCTGAGTAC
GCTTCGCGT;
(SEQ ID NO: 5)
CGCCAGTAGTATGTCCATATACCCAAAGTCGCTTCATTGTAC;
(SEQ ID NO: 6)
CGCCAGTAGTATGTCCATGAATTC;
(SEQ ID NO: 7)
CGCCAGTA;
(SEQ ID NO: 8)
CGCCAG;
(SEQ ID NO: 14)
TCCGGTTGGAATGCAGGACACTCTCCGCAAGGTAGCCTGTTCTTGGCTCT
CTCCCTCCTAGGCACTACGGCCAGGGTGGGTAGCGG;
(SEQ ID NO: 15)
TCCGGTTGGAATGCAGGACACTCTCCGCAAGGTAGCCTGTTCTTGGCTCT
CTCCCTCCTAGGCACTACGGCCAGGGTGGGTA;
(SEQ ID NO: 16)
TCCGGTTGGAATGCAGGACACTCTCCGCAAGGTAGCCTGTTCTTGGCTCT
C;
(SEQ ID NO: 17)
TCCGGTTGGAATGCAGGACACTCTCCGCAAGGTAGCC;
or
(SEQ ID NO: 18)
TCCGGTTGGAATGCAGGACACTCTCCGCAA,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 4. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 5. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 6. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 7. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 8. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 14. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 15. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 16. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 17. In some embodiments, the mutated msd locus upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 18. In some embodiments, the mutated msd locus upstream of the donor DNA sequence comprises the nucleic acid sequence of SEQ ID NO: 4. In some embodiments, the mutated msd locus upstream of the donor DNA sequence comprises the nucleic acid sequence of SEQ ID NO: 5. In some embodiments, the mutated msd locus upstream of the donor DNA sequence comprises the nucleic acid sequence of SEQ ID NO: 6. In some embodiments, the mutated msd locus upstream of the donor DNA sequence comprises the nucleic acid sequence of SEQ ID NO: 7. In some embodiments, the mutated msd locus upstream of the donor DNA sequence comprises the nucleic acid sequence of SEQ ID NO: 8. In some embodiments, the mutated msd locus upstream of the donor DNA sequence comprises the nucleic acid sequence of SEQ ID NO: 14. In some embodiments, the mutated msd locus upstream of the donor DNA sequence comprises the nucleic acid sequence of SEQ ID NO: 15. In some embodiments, the mutated msd locus upstream of the donor DNA sequence comprises the nucleic acid sequence of SEQ ID NO: 16. In some embodiments, the mutated msd locus upstream of the donor DNA sequence comprises the nucleic acid sequence of SEQ ID NO: 17. In some embodiments, the mutated msd locus upstream of the donor DNA sequence comprises the nucleic acid sequence of SEQ ID NO: 18.

In some embodiments, the mature retron comprises a mutated msd reverse transcript upstream (to the 5′ of) of the donor DNA sequence comprises a nucleic acid sequence of:

(SEQ ID NO: 49)
ACGCGAAGCGTACTCAGGTACAATGAAGCGACTTTGGGTATATGGA
CATACTACTGGCG;
(SEQ ID NO: 50)
GTACAATGAAGCGACTTTGGGTATATGGACATACTACTGGCG;
(SEQ ID NO: 51)
GAATTCATGGACATACTACTGGCG;
(SEQ ID NO: 52)
TACTGGCG;
(SEQ ID NO: 53)
CTGGCG;
(SEQ ID NO: 54)
CCGCTACCCACCCTGGCCGTAGTGCCTAGGAGGGAGAGAGCCAAGA
ACAGGCTACCTTGCGGAGAGTGTCCTGCATTCCAACCGGA;
(SEQ ID NO: 55)
TACCCACCCTGGCCGTAGTGCCTAGGAGGGAGAGAGCCAAGAACAG
GCTACCTTGCGGAGAGTGTCCTGCATTCCAACCGGA;
(SEQ ID NO: 56)
GAGAGCCAAGAACAGGCTACCTTGCGGAGAGTGTCCTGCATTCCAA
CCGGA;
(SEQ ID NO: 57)
GGCTACCTTGCGGAGAGTGTCCTGCATTCCAACCGGA;
or
(SEQ ID NO: 58)
TTGCGGAGAGTGTCCTGCATTCCAACCGGA,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 49. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 50. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 51. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 52. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 53. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 54. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 55. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 56. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 57. In some embodiments, the mutated msd reverse transcript upstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 58. In some embodiments, the mutated msd reverse transcript upstream of the donor DNA sequence comprises the nucleic acid sequence of SEQ ID NO: 49. In some embodiments, the mutated msd reverse transcript upstream of the donor DNA sequence comprises the nucleic acid sequence of SEQ ID NO: 50. In some embodiments, the mutated msd reverse transcript upstream of the donor DNA sequence comprises the nucleic acid sequence of SEQ ID NO: 51. In some embodiments, the mutated msd reverse transcript upstream of the donor DNA sequence comprises the nucleic acid sequence of SEQ ID NO: 52. In some embodiments, the mutated msd reverse transcript upstream of the donor DNA sequence comprises the nucleic acid sequence of SEQ ID NO: 53. In some embodiments, the mutated msd reverse transcript upstream of the donor DNA sequence comprises the nucleic acid sequence of SEQ ID NO: 54. In some embodiments, the mutated msd reverse transcript upstream of the donor DNA sequence comprises the nucleic acid sequence of SEQ ID NO: 55. In some embodiments, the mutated msd reverse transcript upstream of the donor DNA sequence comprises the nucleic acid sequence of SEQ ID NO: 56. In some embodiments, the mutated msd reverse transcript upstream of the donor DNA sequence comprises the nucleic acid sequence of SEQ ID NO: 57. In some embodiments, the mutated msd reverse transcript upstream of the donor DNA sequence comprises the nucleic acid sequence of SEQ ID NO: 58.

In some embodiments, the mutated msd locus downstream (to the 3′ of) of the donor DNA sequence comprises a nucleic acid sequence of:

(SEQ ID NO: 9)
GCGCTGACGCGCTCAGTACAGTTACGCGCCTTCGGGATGGTTTAAT
GG;
(SEQ ID NO: 10)
GTACAGTTACGCGCCTTCGGGATGGTTTAATGG;
(SEQ ID NO: 11)
GAATTCATGGTTTAATGG;
(SEQ ID NO: 12)
TAATGG;
(SEQ ID NO: 13)
ATGG;
(SEQ ID NO: 19)
CCGCCGTTTACCCACCCCGGCCGTAGTGCCTAGGAGGGGAGAGCCG
GTGAGGCTACCGTGCCCCCAGGTAAGATGG;
(SEQ ID NO: 20)
TACCCACCCCGGCCGTAGTGCCTAGGAGGGGAGAGCCGGTGAGGCT
ACCGTGCCCCCAGGTAAGATGG;
(SEQ ID NO: 21)
GAGAGCCGGTGAGGCTACCGTGCCCCCAGGTAAGATGG;
(SEQ ID NO: 22)
GGCTACCGTGCCCCCAGGTAAGATGG;
or
(SEQ ID NO: 23)
GTGCCCCCAGGTAAGATGG,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 9. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 10. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 11. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 12. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 13. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 19. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 20. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 21. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 22. In some embodiments, the mutated msd locus downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the mutated msd locus downstream of the donor DNA sequence comprises the nucleic acid sequence of SEQ ID NO: 9. In some embodiments, the mutated msd locus downstream of the donor DNA sequence comprises the nucleic acid sequence of SEQ ID NO: 10. In some embodiments, the mutated msd locus downstream of the donor DNA sequence comprises the nucleic acid sequence of SEQ ID NO: 11. In some embodiments, the mutated msd locus downstream of the donor DNA sequence comprises the nucleic acid sequence of SEQ ID NO: 12. In some embodiments, the mutated msd locus downstream of the donor DNA sequence comprises the nucleic acid sequence of SEQ ID NO: 13. In some embodiments, the mutated msd locus downstream of the donor DNA sequence comprises the nucleic acid sequence of SEQ ID NO: 19. In some embodiments, the mutated msd locus downstream of the donor DNA sequence comprises the nucleic acid sequence of SEQ ID NO: 20. In some embodiments, the mutated msd locus downstream of the donor DNA sequence comprises the nucleic acid sequence of SEQ ID NO: 21. In some embodiments, the mutated msd locus downstream of the donor DNA sequence comprises the nucleic acid sequence of SEQ ID NO: 22. In some embodiments, the mutated msd locus downstream of the donor DNA sequence comprises the nucleic acid sequence of SEQ ID NO: 23.

In some embodiments, the mature retron comprises a mutated msd reverse transcript downstream (to the 3′ of) of the donor DNA sequence comprises a nucleic acid sequence of:

(SEQ ID NO: 59)
CCATTAAACCATCCCGAAGGCGCGTAACTGTACTGAGCGCGTCAGC
GC;
(SEQ ID NO: 60)
CCATTAAACCATCCCGAAGGCGCGTAACTGTAC;
(SEQ ID NO: 61)
CCATTAAACCATGAATTC;
(SEQ ID NO: 62)
CCATTA;
(SEQ ID NO: 63)
CCAT;
(SEQ ID NO: 64)
CCATCTTACCTGGGGGCACGGTAGCCTCACCGGCTCTCCCCTCCTA
GGCACTACGGCCGGGGTGGGTAAACGGCGG;
(SEQ ID NO: 65)
CCATCTTACCTGGGGGCACGGTAGCCTCACCGGCTCTCCCCTCCTA
GGCACTACGGCCGGGGTGGGTA;
(SEQ ID NO: 66)
CCATCTTACCTGGGGGCACGGTAGCCTCACCGGCTCTC;
(SEQ ID NO: 67)
CCATCTTACCTGGGGGCACGGTAGCC;
or
(SEQ ID NO: 68)
CCATCTTACCTGGGGGCAC,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any of the foregoing.

In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 59. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 60. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 61. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 62. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 63. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 64. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 65. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 66. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 67. In some embodiments, the mutated msd reverse transcript downstream of the donor DNA sequence comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 68. In some embodiments, the mutated msd reverse transcript downstream of the donor DNA sequence comprises the nucleic acid sequence of SEQ ID NO: 59. In some embodiments, the mutated msd reverse transcript downstream of the donor DNA sequence comprises the nucleic acid sequence of SEQ ID NO: 60. In some embodiments, the mutated msd reverse transcript downstream of the donor DNA sequence comprises the nucleic acid sequence of SEQ ID NO: 61. In some embodiments, the mutated msd reverse transcript downstream of the donor DNA sequence comprises the nucleic acid sequence of SEQ ID NO: 62. In some embodiments, the mutated msd reverse transcript downstream of the donor DNA sequence comprises the nucleic acid sequence of SEQ ID NO: 63. In some embodiments, the mutated msd reverse transcript downstream of the donor DNA sequence comprises the nucleic acid sequence of SEQ ID NO: 64. In some embodiments, the mutated msd reverse transcript downstream of the donor DNA sequence comprises the nucleic acid sequence of SEQ ID NO: 65. In some embodiments, the mutated msd reverse transcript downstream of the donor DNA sequence comprises the nucleic acid sequence of SEQ ID NO: 66. In some embodiments, the mutated msd reverse transcript downstream of the donor DNA sequence comprises the nucleic acid sequence of SEQ ID NO: 67. In some embodiments, the mutated msd reverse transcript downstream of the donor DNA sequence comprises the nucleic acid sequence of SEQ ID NO: 68.

As used herein, a “fragment” can refer to a sequence (polynucleotide or amino acid) that has 1, 2, 3, 4, 5, 6, 7, 8, 9 or 10 fewer nucleotides or residues, which can be referred to as a deletion. The nucleotides can be removed from either the 5′ end (e.g., 5′ deletion) or the 3′ end (e.g., 3′ deletion) of the recited sequence. In some embodiments, the nucleotides can be removed internally.

In some embodiments, the coding region comprises a nucleic acid molecule comprising a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18 upstream of the donor DNA nucleic acid sequence and a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23 downstream of the donor DNA nucleic acid sequence.

In some embodiments, the coding region comprises a nucleic acid molecule comprising a nucleic acid sequence selected from any one of SEQ ID NO: 4, 5, 6, 7, 8, 14, 15, 16, 17, or 18 upstream of the donor DNA nucleic acid sequence and a nucleic acid sequence selected from any one of SEQ ID NO: 9, 10, 11, 12, 13, 19, 20, 21, 22, or 23 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 4 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 5 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 6 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 7 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 8 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 14 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 15 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 16 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 17 upstream of the donor DNA nucleic acid sequence and a nucleic acid sequence of SEQ ID NO: 23 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 9 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 10 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 11 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 12 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 13 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 19 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 20 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 21 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 22 downstream of the donor DNA nucleic acid sequence. In some embodiments, the coding region comprises a nucleic acid molecule comprising a nucleic acid sequence of SEQ ID NO: 18 upstream of the donor DNA nucleic acid sequence and a nucleic acid sequence of SEQ ID NO: 23 downstream of the donor DNA nucleic acid sequence.

In some embodiments, the coding region comprises a nucleic acid molecule comprising a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 49, 50, 51, 52, 53, 54, 55, 56, 57, or 58 upstream of the donor DNA nucleic acid sequence and a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 509, 60, 61, 62, 63, 64, 65, 66, 67, or 68 downstream of the donor DNA nucleic acid sequence.

In some embodiments, the coding region comprises a nucleic acid molecule comprising a nucleic acid sequence selected from any one of SEQ ID NO: 49, 5, 6, 7, 8, 14, 15, 16, 17, or 18 upstream of the donor DNA nucleic acid sequence and a nucleic acid sequence selected from any one of SEQ ID NO: 59, 10, 11, 12, 13, 19, 20, 21, 22, or 23 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 49 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 50 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 51 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 52 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 53 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 54 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 55 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 56 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 57 upstream of the donor DNA nucleic acid sequence and a nucleic acid sequence of SEQ ID NO: 68 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 59 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 60 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 61 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 62 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 63 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 64 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 65 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 66 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 67 downstream of the donor DNA nucleic acid sequence. In some embodiments, the coding region comprises a nucleic acid molecule comprising a nucleic acid sequence of SEQ ID NO: 58 upstream of the donor DNA nucleic acid sequence and a nucleic acid sequence of SEQ ID NO: 68 downstream of the donor DNA nucleic acid sequence.

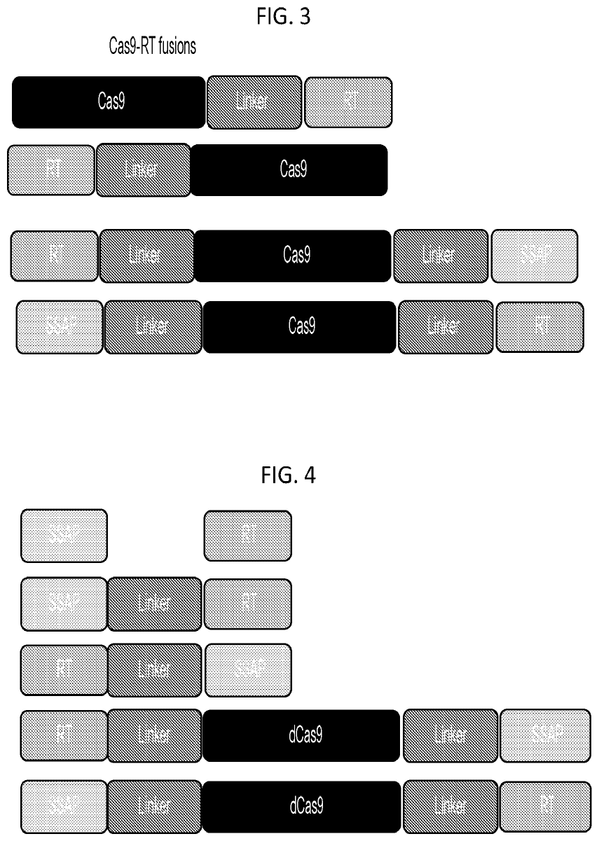

The sequences provided for herein are exemplary in nature and other msr/msd loci can be used and mutated versions thereof. The structure of such mutated loci can be illustrated as shown in FIG. 8A-1 and FIG. 8A-2. These are illustrative in nature only and are not intended to represent the actual topology, folding or 3-dimensional structure of the nucleotide molecule that is transcribed and subject to reverse transcription by a RT. FIG. 8A-1 illustrates the donor DNA sequence (D1) in a msr/msd locus, which includes a RT binding region formed by RT_{1A} and RT_{2A}. The panels in FIG. 8A-1 illustrate various mutants that have deletions in the locus changing the overall length of the msr/msd locus and the position (number of nucleotides away from the 5′ or 3′ end of the transcript) of the donor DNA insert as it relates to the 5′ or 3′ end of the msr/msd locus. Other variants can also be made. FIG. 8A-2 illustrates, non-limiting embodiments that were tested. The panel labeled with 53S is the full length msr/msd locus and the others are various mutations (deletions) of the locus that were found to, surprisingly, increase gene editing efficiency. The various mutant loci are also illustrated in an alignments, which are shown in, for example, FIG. 8A-3 and FIG. 8A-4. These alignments illustrate the different lengths of the loci and the position of the donor DNA insert which is represented by D1 in the figure. The sequences that are double underlined are the msd locus that are present in these non-limiting examples. As can be seen in the examples provided for herein, Homology-Directed Repair (HDR) efficiency can be increased 2-3 fold by modifying the msr/msd locus and the location of the donor DNA insert in the coding region. Accordingly, in some embodiments, the methods comprise increasing HDR by at least 1, 2, 3, 4, or 5 fold with the mutated msr/msd locus, which may just include mutations in the msd locus as provided for herein in non-limiting embodiments.

Thus, in some embodiments, the coding region can be represented by a formula. In some embodiments, the coding region comprises a nucleic acid molecule having a formula of 5′-M1-X1-M2-3′, wherein M1 is a fragment of the msr/msd locus, X1 is the donor DNA sequence; and M2 is a fragment of the msd/msr locus. M1 can, for example, comprise the sequences represented by RT_{1A} and RT_{1B} in FIG. 8A-1. M2 can, for example, comprise the sequences represented by RT_{2A} and RT_{2B} in FIG. 8A-1.

In some embodiments, M1 comprises a sequence of:

(SEQ ID NO: 24)
CGCCAGCAGTGGCAATAGCGTTTCCGGCCTTTTGTGCCGGGAGGGT
CGGCGAGTCGCTGACTTAACGCCAGTAGTATGTCCATATACCCAAA
GTCGCTTCATTGTACCTGAGTACGCTTCGCGT;
(SEQ ID NO: 25)
CGCCAGCAGTGGCAATAGCGTTTCCGGCCTTTTGTGCCGGGAGGGT
CGGCGAGTCGCTGACTTAACGCCAGTAGTATGTCCATATACCCAAA
GTCGCTTCATTGTAC;
(SEQ ID NO: 26)
CGCCAGCAGTGGCAATAGCGTTTCCGGCCTTTTGTGCCGGGAGGGT
CGGCGAGTCGCTGACTTAACGCCAGTAGTATGTCCATGAATTC;
(SEQ ID NO: 27)
CGCCAGCAGTGGCAATAGCGTTTCCGGCCTTTTGTGCCGGGAGGGT
CGGCGAGTCGCTGACTTAACGCCAGTA;
(SEQ ID NO: 28)
CGCCAGCAGTGGCAATAGCGTTTCCGGCCTTTTGTGCCGGGAGGGT
CGGCGAGTCGCTGACTTAACGCCAG;
(SEQ ID NO: 29)
GCGCGAGCAGCCGAGAGAGGTCCGGAGTGCATCAGCCTGAGCGCCT
CGAGCGGCGGAGCGGCGTTGCGCCGCTCCGGTTGGAATGCAGGACA
CTCTCCGCAAGGTAGCCTGTTCTTGGCTCTCTCCCTCCTAGGCACT
ACGGCCAGGGTGGGTAGCGG;
(SEQ ID NO: 30)
GCGCGAGCAGCCGAGAGAGGTCCGGAGTGCATCAGCCTGAGCGCCT
CGAGCGGCGGAGCGGCGTTGCGCCGCTCCGGTTGGAATGCAGGACA
CTCTCCGCAAGGTAGCCTGTTCTTGGCTCTCTCCCTCCTAGGCACT
ACGGCCAGGGTGGGTA;
(SEQ ID NO: 31)
GCGCGAGCAGCCGAGAGAGGTCCGGAGTGCATCAGCCTGAGCGCCT
CGAGCGGCGGAGCGGCGTTGCGCCGCTCCGGTTGGAATGCAGGACA
CTCTCCGCAAGGTAGCCTGTTCTTGGCTCTC;
(SEQ ID NO: 32)
GCGCGAGCAGCCGAGAGAGGTCCGGAGTGCATCAGCCTGAGCGCCT
CGAGCGGCGGAGCGGCGTTGCGCCGCTCCGGTTGGAATGCAGGACA
CTCTCCGCAAGGTAGCC;
or
(SEQ ID NO: 33)
GCGCGAGCAGCCGAGAGAGGTCCGGAGTGCATCAGCCTGAGCGCCT
CGAGCGGCGGAGCGGCGTTGCGCCGCTCCGGTTGGAATGCAGGACA
CTCTCCGCAA,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any of the foregoing.

In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 24. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 25. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 26. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 27. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 28. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 29. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 30. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 31. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 32. In some embodiments, the M1 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 33. In some embodiments, the M1 comprises the nucleic acid sequence of SEQ ID NO: 24. In some embodiments, the M1 comprises the nucleic acid sequence of SEQ ID NO: 25. In some embodiments, the M1 comprises the nucleic acid sequence of SEQ ID NO: 26. In some embodiments, the M1 comprises the nucleic acid sequence of SEQ ID NO: 27. In some embodiments, the M1 comprises the nucleic acid sequence of SEQ ID NO: 28. In some embodiments, the M1 comprises the nucleic acid sequence of SEQ ID NO: 29. In some embodiments, the M1 comprises the nucleic acid sequence of SEQ ID NO: 30. In some embodiments, the M1 comprises the nucleic acid sequence of SEQ ID NO: 31. In some embodiments, the M1 comprises the nucleic acid sequence of SEQ ID NO: 32. In some embodiments, the M1 comprises the nucleic acid sequence of SEQ ID NO: 33.

In some embodiments, M2 comprises a sequence of:

(SEQ ID NO: 34)
GCGCTGACGCGCTCAGTACAGTTACGCGCCTTCGGGATGGTTTAAT
GGTATTGCCGCTGTTGGCG;
(SEQ ID NO: 35)
GTACAGTTACGCGCCTTCGGGATGGTTTAATGGTATTGCCGCTGTT
GGCG;
(SEQ ID NO: 36)
GAATTCATGGTTTAATGGTATTGCCGCTGTTGGCG;
(SEQ ID NO: 37)
TAATGGTATTGCCGCTGTTGGCG;
(SEQ ID NO: 38)
ATGGTATTGCCGCTGTTGGCG;
(SEQ ID NO: 39)
CCGCCGTTTACCCACCCCGGCCGTAGTGCCTAGGAGGGGAGAGCCG
GTGAGGCTACCGTGCCCCCAGGTAAGATGGTGGTGCTTTCCCGGCC
TCCCTCGACTGCTCGCGC;
(SEQ ID NO: 40)
TACCCACCCCGGCCGTAGTGCCTAGGAGGGGAGAGCCGGTGAGGCT
ACCGTGCCCCCAGGTAAGATGGTGGTGCTTTCCCGGCCTCCCTCGA
CTGCTCGCGC;
(SEQ ID NO: 41)
GAGAGCCGGTGAGGCTACCGTGCCCCCAGGTAAGATGGTGGTGCTT
TCCCGGCCTCCCTCGACTGCTCGCGC;
(SEQ ID NO: 42)
GGCTACCGTGCCCCCAGGTAAGATGGTGGTGCTTTCCCGGCCTCCC
TCGACTGCTCGCGC;
(SEQ ID NO: 43)
GTGCCCCCAGGTAAGATGGTGGTGCTTTCCCGGCCTCCCTCGACTG
CTCGCG,

or a fragment of any of the foregoing or a sequence that has at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any of the foregoing.

In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 34. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 35. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 36. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 37. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 38. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 39. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 40. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 41. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 42. In some embodiments, the M2 comprises the nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 43. In some embodiments, the M2 comprises the nucleic acid sequence of SEQ ID NO: 34. In some embodiments, the M2 comprises the nucleic acid sequence of SEQ ID NO: 35. In some embodiments, the M2 comprises the nucleic acid sequence of SEQ ID NO: 36. In some embodiments, the M2 comprises the nucleic acid sequence of SEQ ID NO: 37. In some embodiments, the M2 comprises the nucleic acid sequence of SEQ ID NO: 38. In some embodiments, the M2 comprises the nucleic acid sequence of SEQ ID NO: 39. In some embodiments, the M2 comprises the nucleic acid sequence of SEQ ID NO: 40. In some embodiments, the M2 comprises the nucleic acid sequence of SEQ ID NO: 41. In some embodiments, the M2 comprises the nucleic acid sequence of SEQ ID NO: 42. In some embodiments, the M2 comprises the nucleic acid sequence of SEQ ID NO: 43.

In some embodiments, the nucleic acid molecule comprises an M1 having a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and an M2 having a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.

In some embodiments, the nucleic acid molecule comprises an M1 having a nucleic acid sequence of SEQ ID NO: 24; and an M2 having a nucleic acid sequence of SEQ ID NO: 34. In some embodiments, the nucleic acid molecule comprises an M1 having a nucleic acid sequence of SEQ ID NO: 24; and an M2 having a nucleic acid sequence of SEQ ID NO: 35. In some embodiments, the nucleic acid molecule comprises an M1 having a nucleic acid sequence of SEQ ID NO: 24; and an M2 having a nucleic acid sequence of SEQ ID NO: 36. In some embodiments, the nucleic acid molecule comprises an M1 having a nucleic acid sequence of SEQ ID NO: 24; and an M2 having a nucleic acid sequence of SEQ ID NO: 37. In some embodiments, the nucleic acid molecule comprises an M1 having a nucleic acid sequence of SEQ ID NO: 24; and an M2 having a nucleic acid sequence of SEQ ID NO: 38. In some embodiments, the nucleic acid molecule comprises an M1 having a nucleic acid sequence of SEQ ID NO: 24; and an M2 having a nucleic acid sequence of SEQ ID NO: 39. In some embodiments, the nucleic acid molecule comprises an M1 having a nucleic acid sequence of SEQ ID NO: 24; and an M2 having a nucleic acid sequence of SEQ ID NO: 40. In some embodiments, the nucleic acid molecule comprises an M1 having a nucleic acid sequence of SEQ ID NO: 24; and an M2 having a nucleic acid sequence of SEQ ID NO: 41. In some embodiments, the nucleic acid molecule comprises an M1 having a nucleic acid sequence of SEQ ID NO: 24; and an M2 having a nucleic acid sequence of SEQ ID NO: 42. In some embodiments, the nucleic acid molecule comprises an M1 having a nucleic acid sequence of SEQ ID NO: 24; and an M2 having a nucleic acid sequence of SEQ ID NO: 43. In some embodiments, the nucleic acid molecule comprises an M1 having a nucleic acid sequence of SEQ ID NO: 25; and an M2 having a nucleic acid sequence of SEQ ID NO: 34. In some embodiments, the nucleic acid molecule comprises an M1 having a nucleic acid sequence of SEQ ID NO: 25; and an M2 having a nucleic acid sequence of SEQ ID NO: 35. In some embodiments, the nucleic acid molecule comprises an M1 having a nucleic acid sequence of SEQ ID NO: 25; and an M2 having a nucleic acid sequence of SEQ ID NO: 36. In some embodiments, the nucleic acid molecule comprises an M1 having a nucleic acid sequence of SEQ ID NO: 25; and an M2 having a nucleic acid sequence of SEQ ID NO: 37. In some embodiments, the nucleic acid molecule comprises an M1 having a nucleic acid sequence of SEQ ID NO: 25; and an M2 having a nucleic acid sequence of SEQ ID NO: 38. In some embodiments, the nucleic acid molecule comprises an M1 having a nucleic acid sequence of SEQ ID NO: 25; and an M2 having a nucleic acid sequence of SEQ ID NO: 39. In some embodiments, the nucleic acid molecule comprises an M1 having a nucleic acid sequence of SEQ ID NO: 25; and an M2 having a nucleic acid sequence of SEQ ID NO: 40. In some embodiments, the nucleic acid molecule comprises an M1 having a nucleic acid sequence of SEQ ID NO: 25; and an M2 having a nucleic acid sequence of SEQ ID NO: 41. In some embodiments, the nucleic acid molecule comprises an M1 having a nucleic acid sequence of SEQ ID NO: 25; and an M2 having a nucleic acid sequence of SEQ ID NO: 42. In some embodiments, the nucleic acid molecule comprises an M1 having a nucleic acid sequence of SEQ ID NO: 25; and an M2 having a nucleic acid sequence of SEQ ID NO: 43. In some embodiments, the nucleic acid molecule comprises an M1 having a nucleic acid sequence of SEQ ID NO: 26; and an M2 having a nucleic acid sequence of SEQ ID NO: 34. In some embodiments, the nucleic acid molecule comprises an M1 having a nucleic acid sequence of SEQ ID NO: 26; and an M2 having a nucleic acid sequence of SEQ ID NO: 35. In some embodiments, the nucleic acid molecule comprises an M1 having a nucleic acid sequence of SEQ ID NO: 26; and an M2 having a nucleic acid sequence of SEQ ID NO: 36. In some embodiments, the nucleic acid molecule comprises an M1 having a nucleic acid sequence of SEQ ID NO: 26; and an M2 having a nucleic acid sequence of SEQ ID NO: 37. In some embodiments, the nucleic acid molecule comprises an M1 having a nucleic acid sequence of SEQ ID NO: 26; and an M2 having a nucleic acid sequence of SEQ ID NO: 38. In some embodiments, the nucleic acid molecule comprises an M1 having a nucleic acid sequence of SEQ ID NO: 26; and an M2 having a nucleic acid sequence of SEQ ID NO: 39. In some embodiments, the nucleic acid molecule comprises an M1 having a nucleic acid sequence of SEQ ID NO: 26; and an M2 having a nucleic acid sequence of SEQ ID NO: 40. In some embodiments, the nucleic acid molecule comprises an M1 having a nucleic acid sequence of SEQ ID NO: 26; and an M2 having a nucleic acid sequence of SEQ ID NO: 41. In some embodiments, the nucleic acid molecule comprises an M1 having a nucleic acid sequence of SEQ ID NO: 26; and an M2 having a nucleic acid sequence of SEQ ID NO: 42. In some embodiments, the nucleic acid molecule comprises an M1 having a nucleic acid sequence of SEQ ID NO: 26; and an M2 having a nucleic acid sequence of SEQ ID NO: 43. In some embodiments, the nucleic acid molecule comprises an M1 having a nucleic acid sequence of SEQ ID NO: 27; and an M2 having a nucleic acid sequence of SEQ ID NO: 34. In some embodiments, the nucleic acid molecule comprises an M1 having a nucleic acid sequence of SEQ ID NO: 27; and an M2 having a nucleic acid sequence of SEQ ID NO: 35. In some embodiments, the nucleic acid molecule comprises an M1 having a nucleic acid sequence of SEQ ID NO: 27; and an M2 having a nucleic acid sequence of SEQ ID NO: 36. In some embodiments, the nucleic acid molecule comprises an M1 having a nucleic acid sequence of SEQ ID NO: 27; and an M2 having a nucleic acid sequence of SEQ ID NO: 37. In some embodiments, the nucleic acid molecule comprises an M1 having a nucleic acid sequence of SEQ ID NO: 27; and an M2 having a nucleic acid sequence of SEQ ID NO: 38. In some embodiments, the nucleic acid molecule comprises an M1 having a nucleic acid sequence of SEQ ID NO: 27; and an M2 having a nucleic acid sequence of SEQ ID NO: 39. In some embodiments, the nucleic acid molecule comprises an M1 having a nucleic acid sequence of SEQ ID NO: 27; and an M2 having a nucleic acid sequence of SEQ ID NO: 40. In some embodiments, the nucleic acid molecule comprises an M1 having a nucleic acid sequence of SEQ ID NO: 27; and an M2 having a nucleic acid sequence of SEQ ID NO: 41. In some embodiments, the nucleic acid molecule comprises an M1 having a nucleic acid sequence of SEQ ID NO: 27; and an M2 having a nucleic acid sequence of SEQ ID NO: 42. In some embodiments, the nucleic acid molecule comprises an M1 having a nucleic acid sequence of SEQ ID NO: 27; and an M2 having a nucleic acid sequence of SEQ ID NO: 43. In some embodiments, the nucleic acid molecule comprises an M1 having a nucleic acid sequence of SEQ ID NO: 28; and an M2 having a nucleic acid sequence of SEQ ID NO: 34. In some embodiments, the nucleic acid molecule comprises an M1 having a nucleic acid sequence of SEQ ID NO: 28; and an M2 having a nucleic acid sequence of SEQ ID NO: 35. In some embodiments, the nucleic acid molecule comprises an M1 having a nucleic acid sequence of SEQ ID NO: 28; and an M2 having a nucleic acid sequence of SEQ ID NO: 36. In some embodiments, the nucleic acid molecule comprises an M1 having a nucleic acid sequence of SEQ ID NO: 28; and an M2 having a nucleic acid sequence of SEQ ID NO: 37. In some embodiments, the nucleic acid molecule comprises an M1 having a nucleic acid sequence of SEQ ID NO: 28; and an M2 having a nucleic acid sequence of SEQ ID NO: 38. In some embodiments, the nucleic acid molecule comprises an M1 having a nucleic acid sequence of SEQ ID NO: 28; and an M2 having a nucleic acid sequence of SEQ ID NO: 39. In some embodiments, the nucleic acid molecule comprises an M1 having a nucleic acid sequence of SEQ ID NO: 28; and an M2 having a nucleic acid sequence of SEQ ID NO: 40. In some embodiments, the nucleic acid molecule comprises an M1 having a nucleic acid sequence of SEQ ID NO: 28; and an M2 having a nucleic acid sequence of SEQ ID NO: 41. In some embodiments, the nucleic acid molecule comprises an M1 having a nucleic acid sequence of SEQ ID NO: 28; and an M2 having a nucleic acid sequence of SEQ ID NO: 42. In some embodiments, the nucleic acid molecule comprises an M1 having a nucleic acid sequence of SEQ ID NO: 28; and an M2 having a nucleic acid sequence of SEQ ID NO: 43. In some embodiments, the nucleic acid molecule comprises an M1 having a nucleic acid sequence of SEQ ID NO: 29; and an M2 having a nucleic acid sequence of SEQ ID NO: 34. In some embodiments, the nucleic acid molecule comprises an M1 having a nucleic acid sequence of SEQ ID NO: 29; and an M2 having a nucleic acid sequence of SEQ ID NO: 35. In some embodiments, the nucleic acid molecule comprises an M1 having a nucleic acid sequence of SEQ ID NO: 29; and an M2 having a nucleic acid sequence of SEQ ID NO: 36. In some embodiments, the nucleic acid molecule comprises an M1 having a nucleic acid sequence of SEQ ID NO: 29; and an M2 having a nucleic acid sequence of SEQ ID NO: 37. In some embodiments, the nucleic acid molecule comprises an M1 having a nucleic acid sequence of SEQ ID NO: 29; and an M2 having a nucleic acid sequence of SEQ ID NO: 38. In some embodiments, the nucleic acid molecule comprises an M1 having a nucleic acid sequence of SEQ ID NO: 29; and an M2 having a nucleic acid sequence of SEQ ID NO: 39. In some embodiments, the nucleic acid molecule comprises an M1 having a nucleic acid sequence of SEQ ID NO: 29; and an M2 having a nucleic acid sequence of SEQ ID NO: 40. In some embodiments, the nucleic acid molecule comprises an M1 having a nucleic acid sequence of SEQ ID NO: 29; and an M2 having a nucleic acid sequence of SEQ ID NO: 41. In some embodiments, the nucleic acid molecule comprises an M1 having a nucleic acid sequence of SEQ ID NO: 29; and an M2 having a nucleic acid sequence of SEQ ID NO: 42. In some embodiments, the nucleic acid molecule comprises an M1 having a nucleic acid sequence of SEQ ID NO: 29; and an M2 having a nucleic acid sequence of SEQ ID NO: 43. In some embodiments, the nucleic acid molecule comprises an M1 having a nucleic acid sequence of SEQ ID NO: 30; and an M2 having a nucleic acid sequence of SEQ ID NO: 34. In some embodiments, the nucleic acid molecule comprises an M1 having a nucleic acid sequence of SEQ ID NO: 30; and an M2 having a nucleic acid sequence of SEQ ID NO: 35. In some embodiments, the nucleic acid molecule comprises an M1 having a nucleic acid sequence of SEQ ID NO: 30; and an M2 having a nucleic acid sequence of SEQ ID NO: 36. In some embodiments, the nucleic acid molecule comprises an M1 having a nucleic acid sequence of SEQ ID NO: 30; and an M2 having a nucleic acid sequence of SEQ ID NO: 37. In some embodiments, the nucleic acid molecule comprises an M1 having a nucleic acid sequence of SEQ ID NO: 30; and an M2 having a nucleic acid sequence of SEQ ID NO: 38. In some embodiments, the nucleic acid molecule comprises an M1 having a nucleic acid sequence of SEQ ID NO: 30; and an M2 having a nucleic acid sequence of SEQ ID NO: 39. In some embodiments, the nucleic acid molecule comprises an M1 having a nucleic acid sequence of SEQ ID NO: 30; and an M2 having a nucleic acid sequence of SEQ ID NO: 40. In some embodiments, the nucleic acid molecule comprises an M1 having a nucleic acid sequence of SEQ ID NO: 30; and an M2 having a nucleic acid sequence of SEQ ID NO: 41. In some embodiments, the nucleic acid molecule comprises an M1 having a nucleic acid sequence of SEQ ID NO: 30; and an M2 having a nucleic acid sequence of SEQ ID NO: 42. In some embodiments, the nucleic acid molecule comprises an M1 having a nucleic acid sequence of SEQ ID NO: 30; and an M2 having a nucleic acid sequence of SEQ ID NO: 43. In some embodiments, the nucleic acid molecule comprises an M1 having a nucleic acid sequence of SEQ ID NO: 31; and an M2 having a nucleic acid sequence of SEQ ID NO: 34. In some embodiments, the nucleic acid molecule comprises an M1 having a nucleic acid sequence of SEQ ID NO: 31; and an M2 having a nucleic acid sequence of SEQ ID NO: 35. In some embodiments, the nucleic acid molecule comprises an M1 having a nucleic acid sequence of SEQ ID NO: 31; and an M2 having a nucleic acid sequence of SEQ ID NO: 36. In some embodiments, the nucleic acid molecule comprises an M1 having a nucleic acid sequence of SEQ ID NO: 31; and an M2 having a nucleic acid sequence of SEQ ID NO: 37. In some embodiments, the nucleic acid molecule comprises an M1 having a nucleic acid sequence of SEQ ID NO: 31; and an M2 having a nucleic acid sequence of SEQ ID NO: 38. In some embodiments, the nucleic acid molecule comprises an M1 having a nucleic acid sequence of SEQ ID NO: 31; and an M2 having a nucleic acid sequence of SEQ ID NO: 39. In some embodiments, the nucleic acid molecule comprises an M1 having a nucleic acid sequence of SEQ ID NO: 31; and an M2 having a nucleic acid sequence of SEQ ID NO: 40. In some embodiments, the nucleic acid molecule comprises an M1 having a nucleic acid sequence of SEQ ID NO: 31; and an M2 having a nucleic acid sequence of SEQ ID NO: 41. In some embodiments, the nucleic acid molecule comprises an M1 having a nucleic acid sequence of SEQ ID NO: 31; and an M2 having a nucleic acid sequence of SEQ ID NO: 42. In some embodiments, the nucleic acid molecule comprises an M1 having a nucleic acid sequence of SEQ ID NO: 31; and an M2 having a nucleic acid sequence of SEQ ID NO: 43. In some embodiments, the nucleic acid molecule comprises an M1 having a nucleic acid sequence of SEQ ID NO: 32; and an M2 having a nucleic acid sequence of SEQ ID NO: 34. In some embodiments, the nucleic acid molecule comprises an M1 having a nucleic acid sequence of SEQ ID NO: 32; and an M2 having a nucleic acid sequence of SEQ ID NO: 35. In some embodiments, the nucleic acid molecule comprises an M1 having a nucleic acid sequence of SEQ ID NO: 32; and an M2 having a nucleic acid sequence of SEQ ID NO: 36. In some embodiments, the nucleic acid molecule comprises an M1 having a nucleic acid sequence of SEQ ID NO: 32; and an M2 having a nucleic acid sequence of SEQ ID NO: 37. In some embodiments, the nucleic acid molecule comprises an M1 having a nucleic acid sequence of SEQ ID NO: 32; and an M2 having a nucleic acid sequence of SEQ ID NO: 38. In some embodiments, the nucleic acid molecule comprises an M1 having a nucleic acid sequence of SEQ ID NO: 32; and an M2 having a nucleic acid sequence of SEQ ID NO: 39. In some embodiments, the nucleic acid molecule comprises an M1 having a nucleic acid sequence of SEQ ID NO: 32; and an M2 having a nucleic acid sequence of SEQ ID NO: 40. In some embodiments, the nucleic acid molecule comprises an M1 having a nucleic acid sequence of SEQ ID NO: 32; and an M2 having a nucleic acid sequence of SEQ ID NO: 41. In some embodiments, the nucleic acid molecule comprises an M1 having a nucleic acid sequence of SEQ ID NO: 32; and an M2 having a nucleic acid sequence of SEQ ID NO: 42. In some embodiments, the nucleic acid molecule comprises an M1 having a nucleic acid sequence of SEQ ID NO: 32; and an M2 having a nucleic acid sequence of SEQ ID NO: 43. In some embodiments, the nucleic acid molecule comprises an M1 having a nucleic acid sequence of SEQ ID NO: 33; and an M2 having a nucleic acid sequence of SEQ ID NO: 34. In some embodiments, the nucleic acid molecule comprises an M1 having a nucleic acid sequence of SEQ ID NO: 33; and an M2 having a nucleic acid sequence of SEQ ID NO: 35. In some embodiments, the nucleic acid molecule comprises an M1 having a nucleic acid sequence of SEQ ID NO: 33; and an M2 having a nucleic acid sequence of SEQ ID NO: 36. In some embodiments, the nucleic acid molecule comprises an M1 having a nucleic acid sequence of SEQ ID NO: 33; and an M2 having a nucleic acid sequence of SEQ ID NO: 37. In some embodiments, the nucleic acid molecule comprises an M1 having a nucleic acid sequence of SEQ ID NO: 33; and an M2 having a nucleic acid sequence of SEQ ID NO: 38. In some embodiments, the nucleic acid molecule comprises an M1 having a nucleic acid sequence of SEQ ID NO: 33; and an M2 having a nucleic acid sequence of SEQ ID NO: 39. In some embodiments, the nucleic acid molecule comprises an M1 having a nucleic acid sequence of SEQ ID NO: 33; and an M2 having a nucleic acid sequence of SEQ ID NO: 40. In some embodiments, the nucleic acid molecule comprises an M1 having a nucleic acid sequence of SEQ ID NO: 33; and an M2 having a nucleic acid sequence of SEQ ID NO: 41. In some embodiments, the nucleic acid molecule comprises an M1 having a nucleic acid sequence of SEQ ID NO: 33; and an M2 having a nucleic acid sequence of SEQ ID NO: 42. In some embodiments, the nucleic acid molecule comprises an M1 having a nucleic acid sequence of SEQ ID NO: 33; and an M2 having a nucleic acid sequence of SEQ ID NO: 43.

In some embodiments of the methods, the host cell expresses (e.g., heterologously expresses) a nuclease and a RT protein. In some embodiments, the nuclease and RT are not expressed as a fusion molecule. In some embodiments, the nuclease and RT are each expressed as separate molecules. In some embodiments, the nuclease and RT are expressed as a fusion protein. In some embodiments, the nuclease is linked to the RT with a linker. In some embodiments, the C-terminus of the nuclease is linked to the N-terminus of the RT. In some embodiments, the N-terminus of the nuclease is linked to the C-terminus of the RT. In some embodiments, the nuclease is a Cas nuclease, such as a Cas9 nuclease. In some embodiments, the Cas nuclease is a catalytically active nuclease, a nickase Cas nuclease, or a catalytically inactive Cas nuclease. In some embodiments, the nickase Cas nuclease is Cas9D10A or Cas9H840A nuclease or a Cas having the both D10A and H840A mutations. The nickase can be other forms and these are illustrative in nature and non-limiting.

In some embodiments, the donor DNA sequence is a target strand donor DNA sequence. In some embodiments, the donor DNA sequence is a non-target strand donor DNA sequence. In some embodiments, the donor DNA sequence is a target strand donor DNA sequence, provided that the Cas nuclease is a catalytically active nuclease, or a nickase Cas nuclease, such as those provided herein. In some embodiments, the donor DNA sequence is a target strand donor DNA sequence, provided that the nickase Cas nuclease is Cas9D10A or Cas9H840A nuclease or a Cas having the both D10A and H840A mutations.

In some embodiments of the methods, the cell is transfected with a vector (e.g, mRNA, DNA, or RNA, plasmid, virus, and the like) expressing the nuclease and/or the RT. In some embodiments, the cell stably expresses the nuclease and/or the RT. In some embodiments, the cell expresses a RT and a single-stranded annealing protein (SSAP).

In some embodiments, the RT and the SSAP are not expressed as a fusion molecule. In some embodiments, the RT and the SSAP are each expressed as separate molecules. In some embodiments, the RT and the SSAP are expressed as a fusion molecule. In some embodiments, the RT and the SSAP are linked to one another with a linker. In some embodiments, the linker is a peptide linker. In some embodiments, the C-terminus of the SSAP is linked to the N-terminus of the RT. In some embodiments, the N-terminus of the SSAP is linked to the C-terminus of the RT.

In some embodiments, the RT is a fusion protein. In some embodiments, the fusion protein comprises a polypeptide having the formula of: of (N₁)_{q}-C₁-L₁-(N₂)_{qq}-R₁-(N₃)_{qqq}, wherein C1 is a nuclease, such as Cas (e.g., CAS9), L1 is a peptide linker, R1 is a RT protein, and N1 and N2 are each, independently, a NLS sequence, wherein q, qq, or qqq, are each, independently, 0, 1, 2, or 3. In some embodiments q is 1 or 2. In some embodiments, when q is 2, the NLS sequences, which can be the same or different, can be separated by a linker sequence. In some embodiments, qqq is 1 or 2. In some embodiments, when qqq is 2, the NLS sequences, which can be the same or different, can be separated by a linker sequence. In some embodiments, qq is 0 or 1. In some embodiments, when qq is 1, the NLS can comprise a linker sequence at the C-terminal end of the sequence and N-terminal to R₁.

In some embodiments, the polypeptide has the formula of:

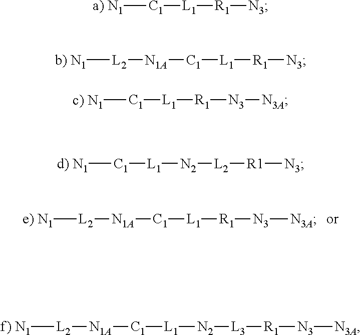

wherein:
   each N1, N1A, N2, N3, or N3A is, independently, a nuclear localization sequence (NLS);
   each L1, L2, or L3 is independently, a linker amino acid sequence; and
wherein each NLS can be the same or different.

In some embodiments, N1, N_{1A}, N₂, N₃, or N_{3A}, each comprise, independently a SV40 NLS sequence, a cMyc NLS sequence, or a Nuceloplasmin NLS sequence. In some embodiments, N₁ or N_{1A} comprises a cMyc or SV40 NLS sequence. In some embodiments, N₂ comprises a SV40 NLS sequence or a Nuceloplasmin NLS sequence. In some embodiments, N₃ or N_{3A} comprises a Nuceloplasmin NLS sequence.

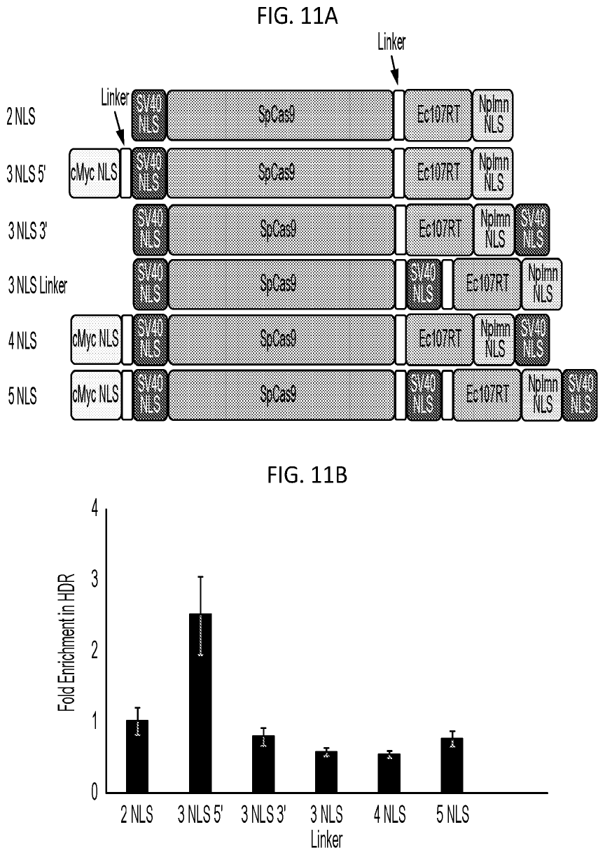

In some embodiments, the polypeptide has a formula as depicted in the polypeptides illustrated in FIG. 11A. FIG. 11A illustrates various domains that can be linked through a linker or without a linker to one another to produce a protein that is transported to the nuclease through the NLS signals that are present in the sequence. FIG. 11A illustrates the Cas nuclease linked to the RT via a peptide linker. In some embodiments, in addition to a peptide linker the nuclease is linked to the RT through a linker and NLS signal, where the NLS sequence is linked to the nuclease directly or through a linker to its N-terminus and to the RT directly or through a linker to its C-terminus. The N- and C-termini can refer to the domain or the entire protein as context dictates. Although, a nuclease, such as CAS9 is illustrated, any other nuclease as provided for herein can be substituted for the nuclease and the RT can be replaced with other RT enzymes. The NLS signals are non-limiting options and other NLS sequences can be utilized.

In some embodiments, L1, L2, or L3, or the linker are each, independently, an XTEN linker, a G/S linker, or a G/A linker, or any combination thereof.

In some embodiments, the Cas comprises a sequence of

(SEQ ID NO: 69)
DKKYSIGLDIGTNSVGWAVITDEYKVPSKKFKVLGNTDRHSIKKNL
IGALLFDSGETAEATRLKRTARRRYTRRKNRICYLQEIFSNEMAKV
DDSFFHRLEESFLVEEDKKHERHPIFGNIVDEVAYHEKYPTIYHLR
KKLVDSTDKADLRLIYLALAHMIKFRGHFLIEGDLNPDNSDVDKLF
IQLVQTYNQLFEENPINASGVDAKAILSARLSKSRRLENLIAQLPG
EKKNGLFGNLIALSLGLTPNFKSNFDLAEDAKLQLSKDTYDDDLDN
LLAQIGDQYADLFLAAKNLSDAILLSDILRVNTEITKAPLSASMIK
RYDEHHQDLTLLKALVRQQLPEKYKEIFFDQSKNGYAGYIDGGASQ
EEFYKFIKPILEKMDGTEELLVKLNREDLLRKQRTFDNGSIPHQIH
LGELHAILRRQEDFYPFLKDNREKIEKILTFRIPYYVGPLARGNSR
FAWMTRKSEETITPWNFEEVVDKGASAQSFIERMTNFDKNLPNEKV
LPKHSLLYEYFTVYNELTKVKYVTEGMRKPAFLSGEQKKAIVDLLF
KTNRKVTVKQLKEDYFKKIECFDSVEISGVEDRFNASLGTYHDLLK
IIKDKDFLDNEENEDILEDIVLTLTLFEDREMIEERLKTYAHLFDD
KVMKQLKRRRYTGWGRLSRKLINGIRDKQSGKTILDFLKSDGFANR
NFMQLIHDDSLTFKEDIQKAQVSGQGDSLHEHIANLAGSPAIKKGI
LQTVKVVDELVKVMGRHKPENIVIEMARENQTTQKGQKNSRERMKR
IEEGIKELGSQILKEHPVENTQLQNEKLYLYYLQNGRDMYVDQELD
INRLSDYDVDHIVPQSFLKDDSIDNKVLTRSDKNRGKSDNVPSEEV
VKKMKNYWRQLLNAKLITQRKFDNLTKAERGGLSELDKAGFIKRQL
VETRQITKHVAQILDSRMNTKYDENDKLIREVKVITLKSKLVSDFR
KDFQFYKVREINNYHHAHDAYLNAVVGTALIKKYPKLESEFVYGDY
KVYDVRKMIAKSEQEIGKATAKYFFYSNIMNFFKTEITLANGEIRK
RPLIETNGETGEIVWDKGRDFATVRKVLSMPQVNIVKKTEVQTGGF
SKESILPKRNSDKLIARKKDWDPKKYGGFDSPTVAYSVLVVAKVEK
GKSKKLKSVKELLGITIMERSSFEKNPIDFLEAKGYKEVKKDLIIK
LPKYSLFELENGRKRMLASAGELQKGNELALPSKYVNFLYLASHYE
KLKGSPEDNEQKQLFVEQHKHYLDEIIEQISEFSKRVILADANLDK
VLSAYNKHRDKPIREQAENIIHLFTLTNLGAPAAFKYFDTTIDRKR
YTSTKEVLDATLIHQSITGLYETRIDLSQLGGD.

In some embodiments, the Cas comprises an amino acid sequence having at least 50%, at least 55%, at least 60%, at least 65%, at least 70%, at least 75%, at least 80%, at least 85%, at least 90%, at least 91%, at least 920%, at least 93%, at least 94%, at least 95%, at least 96%, at least 97%, at least 98%, or at least 99% sequence identity to SEQ ID NO: 69.

In some embodiments, the fusion polypeptide, such as those illustrated in FIG. 11A comprises an amino acid sequence of:

(SEQ ID NO: 70)
MDYKDHDGDYKDHDIDYKDDDDKPAAKRVKLDGGKRTADGSEFESMAPKKKRKVGIHGVPAADKKYSIGLD
IGTNSVGWAVITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFKKIECFDSV
EISGVEDRFNASLGTYHDLLKIIKDKDELDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLDATLIHQSITGLYETR
IDLSQLGGDSGGSSGGSSGSETPGTSESATPESSGGSSGGSSDATRTTLLALDLFGSPGWSADKEIQRLHA
LSNHAGRHYRRIILSKRHGGQRLVLAPDYLLKTVQRNILKNVLSQFPLSPFATAYRPGCPIVSNAQPHCQQ
PQILKLDIENFFDSISWLQVWRVFRQAQLPRNVVTMLTWICCYNDALPQGAPTSPAISNLVMRRFDERIGE
WCQARGITYTRYCDDMTFSGHFNARQVKNKVCGLLAELGLSLNKRKGCLIAACKRQQVTGIVVNHKPQLAR
EARRALRQEVHLCQKYGVISHLSHRGELDPSGDLHAQATAYLYALQGRINWLLQINPEDEAFQQARESVKR
MLVAWKRPAATKKAGQAKKKK;
(SEQ ID NO: 71)
MDYKDHDGDYKDHDIDYKDDDDKMAPKKKRKVGIHGVPAADKKYSIGLDIGTNSVGWAVITDEYKVPSKKF
KVLGNTDRHSIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDSGGSSGGSSGSET
PGTSESATPESSGGSSGGSSDATRTTLLALDLFGSPGWSADKEIQRLHALSNHAGRHYRRIILSKRHGGQR
LVLAPDYLLKTVQRNILKNVLSQFPLSPFATAYRPGCPIVSNAQPHCQQPQILKLDIENFFDSISWLQVWR
VFRQAQLPRNVVTMLTWICCYNDALPQGAPTSPAISNLVMRRFDERIGEWCQARGITYTRYCDDMTFSGHF
NARQVKNKVCGLLAELGLSLNKRKGCLIAACKRQQVTGIVVNHKPQLAREARRALRQEVHLCQKYGVISHL
SHRGELDPSGDLHAQATAYLYALQGRINWLLQINPEDEAFQQARESVKRMLVAWKRPAATKKAGQAKKKKP
KKKRKV;
(SEQ ID NO: 72)
MDYKDHDGDYKDHDIDYKDDDDKPAAKRVKLDGGKRTADGSEFESMAPKKKRKVGIHGVPAADKKYSIGLD
IGTNSVGWAVITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLDATLIHQSITGLYETR
IDLSQLGGDSGGSSGGSSGSETPGTSESATPESSGGSSGGSSDATRTTLLALDLFGSPGWSADKEIQRLHA
LSNHAGRHYRRIILSKRHGGQRLVLAPDYLLKTVQRNILKNVLSQFPLSPFATAYRPGCPIVSNAQPHCQQ
PQILKLDIENFFDSISWLQVWRVFRQAQLPRNVVTMLTWICCYNDALPQGAPTSPAISNLVMRRFDERIGE
WCQARGITYTRYCDDMTFSGHFNARQVKNKVCGLLAELGLSLNKRKGCLIAACKRQQVTGIVVNHKPQLAR
EARRALRQEVHLCQKYGVISHLSHRGELDPSGDLHAQATAYLYALQGRINWLLQINPEDEAFQQARESVKR
MLVAWKRPAATKKAGQAKKKKPKKKRKV;
(SEQ ID NO: 73)
MDYKDHDGDYKDHDIDYKDDDDKMAPKKKRKVGIHGVPAADKKYSIGLDIGTNSVGWAVITDEYKVPSKKF
KVLGNTDRHSIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDSGGSSGGSKRTAD
GSEFESPKKKRKVSGGSSGGSSGGSSGGSSGSETPGTSESATPESSGGSSGGSSDATRTTLLALDLFGSPG
WSADKEIQRLHALSNHAGRHYRRIILSKRHGGQRLVLAPDYLLKTVQRNILKNVLSQFPLSPFATAYRPGC
PIVSNAQPHCQQPQILKLDIENFFDSISWLQVWRVFRQAQLPRNVVTMLTWICCYNDALPQGAPTSPAISN
LVMRRFDERIGEWCQARGITYTRYCDDMTFSGHFNARQVKNKVCGLLAELGLSLNKRKGCLIAACKRQQVT
GIVVNHKPQLAREARRALRQEVHLCQKYGVISHLSHRGELDPSGDLHAQATAYLYALQGRINWLLQINPED
EAFQQARESVKRMLVAWKRPAATKKAGQAKKKK;
or
(SEQ ID NO: 74)
MDYKDHDGDYKDHDIDYKDDDDKPAAKRVKLDGGKRTADGSEFESMAPKKKRKVGIHGVPAADKKYSIGLD
IGTNSVGWAVITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLDATLIHQSITGLYETR
IDLSQLGGDSGGSSGGSKRTADGSEFESPKKKRKVSGGSSGGSSGGSSGGSSGSETPGTSESATPESSGGS
SGGSSDATRTTLLALDLFGSPGWSADKEIQRLHALSNHAGRHYRRIILSKRHGGQRLVLAPDYLLKTVQRN
ILKNVLSQFPLSPFATAYRPGCPIVSNAQPHCQQPQILKLDIENFFDSISWLQVWRVFRQAQLPRNVVTML
TWICCYNDALPQGAPTSPAISNLVMRRFDERIGEWCQARGITYTRYCDDMTFSGHFNARQVKNKVCGLLAE
LGLSLNKRKGCLIAACKRQQVTGIVVNHKPQLAREARRALRQEVHLCQKYGVISHLSHRGELDPSGDLHAQ
ATAYLYALQGRINWLLQINPEDEAFQQARESVKRMLVAWKRPAATKKAGQAKKKKPKKKRKV,

or a variant polypeptide that is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical thereto.

In some embodiments, the fusion polypeptide comprises an amino acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74. In some embodiments, the fusion polypeptide comprises an amino acid sequence of SEQ ID NO: 70. In some embodiments, the fusion polypeptide comprises an amino acid sequence of SEQ ID NO: 71. In some embodiments, the fusion polypeptide comprises an amino acid sequence of SEQ ID NO: 72. In some embodiments, the fusion polypeptide comprises an amino acid sequence of SEQ ID NO: 73. In some embodiments, the fusion polypeptide comprises an amino acid sequence of SEQ ID NO: 74.

In some embodiments, the variant polypeptide comprises a domain that encodes or has RT activity and/or nuclease activity. In some embodiments, the variant polypeptide has at least 80, 85, 90, 91, 92, 93, 94, 95, 96, 97, 98, 99% identity to the sequences recited therein provided that the polypeptide has 1, 2, 3, or 4 NLS amino acid sequences. In some embodiments, the variant polypeptide has at least 80, 85, 90, 91, 92, 93, 94, 95, 96, 97, 98, 99% identity to the sequences recited therein provided that the polypeptide comprises a RT enzyme, which is catalytically active. In some embodiments, the variant polypeptide has at least 80, 85, 90, 91, 92, 93, 94, 95, 96, 97, 98, 99% identity to the sequences recited therein provided that the polypeptide comprises a nuclease, such as a nuclease provided for herein.

In some embodiments of the methods, the retron transcript and the gRNA are not linked together. In some embodiments of the methods, the retron transcript and the gRNA are linked together. In some embodiments, the retron and gRNA are covalently linked together. In some embodiments, the retron transcript and gRNA are linked together with a linker. In some embodiments, the linker comprises a nucleotide sequence, such as nucleotide repeat sequence. In some embodiments, the linker comprises a sequence of (GAA)n, wherein n is 1-20, 1-15, 1-10, 1-5, 1-3, any integer the foregoing ranges, including the endpoints of the range.

In some embodiments, the retron transcript and the gRNA comprises a structured motif at the 5′ or 3′ end of the retron-gRNA transcript to stabilize the transcript when they are part of the same transcript. In some embodiments, the structured motif is, independently, evoPreQ, tevopreQ mpKnot, or exoribonuclease-resistant RNA motifs and/or comprises a nucleotide sequence as set forth herein. In some embodiments, the structured motif is at the 5′ end of the retron-gRNA transcript. In some embodiments, the structured motif is at the 3′ end of the retron-gRNA transcript when they are part of the same transcript. In some embodiments, the structured motif is selected from one of the nucleic acid molecule as set forth in the following table:

| Name | Description | Sequence |
|---|---|---|
| evoPreQ | prequeosine1-1 | TTGACGCGGTTCTAT |
| | riboswitch | CTAGTTACGCGTTAA |
| | aptamer | ACCAACTAGAAA |
| | | (SEQ ID NO: 75) |
| tevopreQ | Trimmed version | CGCGGTTCTATCTAG |
| | of evoPreQ | TTACGCGTTAAACCA |
| | | ACTAGAA |
| | | (SEQ ID NO: 76) |
| mpKnot | pseudoknot from | GGGTCAGGAGCCCCC |
| | Moloney murine | CCCCTGAACCCAGGA |
| | leukemia virus | TAACCCTCAAAGTCG |
| | (MMLV) | GGGGGCAACCC |
| | | (SEQ ID NO: 77) |
| MVE | viral | TAGTCAGGCCAGCCG |
| xrRNA | exoribonuclease- | GTTAGGCTGCCACCG |
| | resistant RNA | AAGGTTGGTAGACGG |
| | motif (xrRNA) | TGCTGCCTGCGACCA |
| | from Murray | ACCCCAGGAGGACTG |
| | Valley | GGT |
| | encephalitis | (SEQ ID NO: 78) |
| WNV | viral | AGTCAGGCCAGATTA |
| xRNA | exoribonuclease- | ATGCTGCCACCGGAA |
| | resistant RNA | GTTGAGTAGACGGTG |
| | motif (xrRNA) | CTGCCTGCGGCTCAA |
| | from West Nile | CCCCAGGAGGACTGG |
| | virus | GT |
| | | (SEQ ID NO: 79) |
| Zika | viral | TGTCAGGCCTGCTAG |
| xRNA | exoribonuclease- | TCAGCCACAGTTTGG |
| | resistant RNA | GGAAAGCTGTGCAGC |
| | motif (xrRNA) | CTGTAACCCCCCCAG |
| | from Zika | GAGAAGCTGGGAAAC |
| | | CAAGCT |
| | | (SEQ ID NO: 80) |
| Dengue | viral | AGTCAGGCCACTTGT |
| xRNA | exoribonuclease- | GCCACGGTTTGAGCA |
| | resistant RNA | AACCGTGCTGCCTGT |
| | motif (xrRNA) | AGCTCCGCCAATAAT |
| | from Dengue | GGGAGGCGT |
| | | (SEQ ID NO: 81) |
| YF | viral | TGTCAGCCCAGAACC |
| | exoribonuclease- | CCACACGAGTTTTGC |
| | resistant RNA | CACTGCTAAGCTGTG |
| | motif (xrRNA) | AGGCAGTGCAGGCTG |
| | from Yellow | GGACAGCCGACCTCC |
| | Fever | AGGTTGCGAAAAACC |
| | | TGGT |
| | | (SEQ ID NO: 82) |

In some embodiments, the retron transcript-guide RNA (gRNA) molecule comprises a molecule of the formula A1-Rt-L₁ₙ-gRNA-A2, wherein:
A1 is absent or a structured motif to stabilize the transcript;
Rt is the retron transcript;
L₁ₙ is absent or a nucleotide linker from about 5 to about 40, about 8 to about 35, about 9, or about 33 basepairs;
gRNA is the guide RNA; and
A2 is absent or a structured motif to stabilize the transcript.

In some embodiments, A1 is a structured motif to stabilize the transcript; Rt is the retron transcript; L₁ₙ is a nucleotide linker from about 5 to about 40, about 8 to about 35, about 9, or about 33 basepairs; gRNA is the guide RNA; and A2 is absent.

In some embodiments, A1 is absent; Rt is the retron transcript; L1 is a nucleotide linker from about 5 to about 40, about 8 to about 35, about 9, or about 33 base pairs; gRNA is the guide RNA; and A2 is a structured motif to stabilize the transcript.

Any number of RTs may be used in alternative embodiments of the present invention, including prokaryotic and eukaryotic RTs. If desired, the nucleotide sequence of a native RT may be modified, for example using known codon optimization techniques, so that expression within the desired host is optimized. By codon optimization it is meant the selection of appropriate DNA nucleotides for the synthesis of oligonucleotide building blocks, and their subsequent enzymatic assembly, of a structural gene or fragment thereof in order to approach codon usage within the host.

The RT may be targeted to the nucleus so that efficient utilization of the RNA template may take place. An example of such a RT includes any known RT, either prokaryotic or eukaryotic, fused to a nuclear localization sequence or signal (NLS). In some embodiments of vectors of the present invention, the vector further comprises an NLS. In particular embodiments of vectors of the present invention, the NLS is located 5′ of the RT coding sequence. Any suitable NLS may also be used, providing that the NLS assists in localizing the RT within the nucleus. The use of an RT in the absence of an NLS may also be used if the RT is present within the nuclear compartment at a level that synthesizes a product from the RNA template.

Retrons are also described in U.S. Pat. No. 8,932,860 and Lampson, et al. Cytogenet. Res. 110:491-499 (2005); both of which are incorporated herein by reference in their entirety for all purposes.

Provided herein are compounds and compositions for editing a genome of a cell.

Guide RNA (gRNA) Molecules

The retron-guide RNA cassettes and retron donor DNA-guide molecules of the present invention comprise guide RNA (gRNA) coding regions and gRNA molecules, respectively. The gRNAs for use in the CRISPR-retron system typically include a crRNA sequence that is complementary to a target nucleic acid sequence and may include a scaffold sequence (e.g., tracrRNA) that interacts with a Cas nuclease (e.g., Cas9) or a variant or fragment thereof, depending on the particular nuclease being used.

The gRNA can comprise any nucleic acid sequence having sufficient complementarity with a target polynucleotide sequence (e.g., target DNA sequence) to hybridize with the target sequence and direct sequence-specific binding of a nuclease to the target sequence. The gRNA may recognize a protospacer adjacent motif (PAM) sequence that may be near or adjacent to the target DNA sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (i.e., be located 5′ of) a 5′NGG PAM, wherein N is any nucleotide, such that the guide sequence of the DNA-targeting RNA (e.g., gRNA) base pairs with the opposite strand to mediate cleavage at about 3 base pairs upstream of the PAM sequence.

In other instances, the target DNA site may lie immediately 3′ of a PAM sequence, e.g., when the Cpf1 endonuclease is used. In some embodiments, the PAM sequence is 5′-TTTN, where N is any nucleotide. When using the Cpf1 endonuclease, the target DNA sequence (i.e., the genomic DNA sequence having complementarity for the gRNA) will typically follow (i.e., be located 3′ of) the PAM sequence. Two CP1-family nucleases, AsCpf1 (from Acidaminococcus) and LbCpf1 (from Lachnospiraceae) are known to function in human cells. Both AsCpf1 and LbCpf1 cut 19 bp after the PAM sequence on the targeted strand and 23 bp after the PAM sequence on the opposite strand of the DNA molecule.

In some embodiments, the degree of complementarity between a guide sequence of the gRNA (i.e., crRNA sequence) and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a crRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a crRNA sequence is about 20 nucleotides in length. In other instances, a crRNA sequence is about 15 nucleotides in length. In other instances, a crRNA sequence is about 25 nucleotides in length.

The nucleotide sequence of a modified gRNA can be selected using any of the web-based software described above. Considerations for selecting a DNA-targeting RNA include the PAM sequence for the nuclease (e.g., Cas9 or Cpf1) to be used, and strategies for minimizing off-target modifications. Tools, such as the CRISPR Design Tool, can provide sequences for preparing the gRNA, for assessing target modification efficiency, and/or assessing cleavage at off-target sites.

In some embodiments, the length of the gRNA molecule is about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more nucleotides in length. In some instances, the length of the gRNA is about 100 nucleotides in length. In other instances, the gRNA is about 90 nucleotides in length. In other instances, the gRNA is about 110 nucleotides in length.

### Donor DNA Sequences

In one aspect, the present embodiments provides retron-guide RNA cassettes comprising a retron that comprises a donor DNA sequence. In another aspect, the present invention provides retron donor DNA-guide molecules comprising retron transcripts that comprise donor DNA sequence coding regions, the retron transcripts subsequently being reverse transcribed to yield msDNA that comprises a donor DNA sequence. The donor DNA sequence or sequences participate in homology-directed repair (HDR) of genetic loci of interest following cleavage of genomic DNA at the genetic locus or loci of interest (i.e., after a nuclease has been directed to cut at a specific genetic locus of interest, targeted by binding of gRNA to a target sequence).

In some embodiments, the recombinant donor repair template (i.e., donor DNA sequence) comprises two homology arms that are homologous to portions of the sequence of the genetic locus of interest at either side of a Cas nuclease (e.g., Cas9 or Cpf1 nuclease) cleavage site. The homology arms may be the same length or may have different lengths. In some instances, each homology arm has at least 50% to at least 99% similarity (i.e., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% similarity) to a portion of the sequence of the genetic locus of interest at either side of a nuclease (e.g., Cas nuclease) cleavage site. In other embodiments, the recombinant donor repair template comprises or further comprises a reporter unit that includes a nucleotide sequence encoding a reporter polypeptide (e.g., a detectable polypeptide, fluorescent polypeptide, or a selectable marker). If present, the two homology arms can flank the reporter cassette and are homologous to portions of the genetic locus of interest at either side of the Cas nuclease cleavage site. The reporter unit can further comprise a sequence encoding a self-cleavage peptide, one or more nuclear localization signals, and/or a fluorescent polypeptide (e.g., superfolder GFP (sfGFP)). Other suitable reporters are described herein. In some embodiments, the donor DNA sequence may be used to introduce a mutation, introduce a new gene, activate a gene, or silence a gene. In some embodiments, the mutation is an insertion, substitution, and/or deletion.

In some embodiments, the donor DNA sequence is at least about 500 to 10,000 (i.e., at least about 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, 5,000, 5,500, 6,000, 6,500, 7,000, 7,500, 8,000, 8,500, 9,000, 9,500, or 10,000) nucleotides in length. In some embodiments, the donor DNA sequence is about 500 to about 10,000 (i.e., about 500, about 600, about 700, about 800, about 900, about 1,000, about 1,100, about 1,200, about 1,300, about 1,400, about 1,500, about 1,600, about 1,700, about 1,800, about 1,900, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, or about 10,000) nucleotides in length. In some embodiments, the donor DNA sequence is at least 500 to at least 10,000 (i.e., at least 500, at least 600, at least 700, at least 800, at least 900, at least 1,000, at least 1,100, at least 1,200, at least 1,300, at least 1,400, at least 1,500, at least 1,600, at least 1,700, at least 1,800, at least 1,900, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, or at least 10,000) nucleotides in length. In some embodiments, the donor DNA sequence is 500 to 10,000 (i.e., 500, 600, 700, 800, 900, 1,000, 1, 100, 1,200, 1,300, 1,400, 1,500, 1,600, 1,700, 1,800, 1,900, 2,000, 2,500, 3,000, 3,500, 4,000, 4,500, 5,000, 5,500, 6,000, 6,500, 7,000, 7,500, 8,000 8,500, 9,000, 9,500, or 10,000) nucleotides in length. In some embodiments, the donor DNA sequence is between about 600 and 1,000 (i.e., about 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000) nucleotides in length.

In some embodiments, the donor DNA sequence is between about 100 and about 500 (i.e., about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500) nucleotides in length. In some embodiments, the donor DNA sequence is between at least 100 and at least 500 (i.e., at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, or at least 500) nucleotides in length. In some embodiments, the donor DNA sequence is between 100 and 500 (i.e., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500) nucleotides in length.

In some embodiments, the donor DNA sequence is about 100 (i.e., about 100, about 95, about 90, about 85, about 80, about 75, about 70, about 65, about 60, about 55, about 50, about 45, about 40, about 35, about 30, about 25, about 20, about 15, about 10, or about 5) nucleotides in length. In some embodiments, the donor DNA sequence is less than 100 (i.e., less than 100, less than 95, less than 90, less than 85, less than 80, less than 75, less than 70, less than 65, less than 60, less than 55, less than 50, less than 45, less than 40, less than 35, less than 30, less than 25, less than 20, less than 15, less than 10, or less than 5) nucleotides in length. In some embodiments, the donor DNA sequence is 100 (i.e., 100, 95, 90, 85, 80, 75, 70, 65, 60, 55, 50, 45, 40, 35, 30, 25, 20, 15, 10, or 5) nucleotides in length.

### CRISPR/Cas System

The CRISPR/Cas system of genome modification includes a Cas nuclease (e.g., Cas9 or Cpf1 nuclease) or a variant or fragment or combination thereof and a DNA-targeting RNA (e.g., guide RNA (gRNA)). The gRNA may contain a guide sequence that targets the Cas nuclease to the target genomic DNA and a scaffold sequence that interacts with the Cas nuclease (e.g., tracrRNA). The system may optionally include a donor repair template. In other instances, a fragment of a Cas nuclease or a variant thereof with desired properties (e.g., capable of generating single- or double-strand breaks and/or modulating gene expression) can be used. The donor repair template can include a nucleotide sequence encoding a reporter polypeptide such as a fluorescent protein or an antibiotic resistance marker, and homology arms that are homologous to the target DNA and flank the site of gene modification.

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease may require both the crRNA and the tracrRNA for site-specific DNA recognition and cleavage. This system has now been engineered such that the crRNA and tracrRNA, if needed, can be combined into one molecule (the “single guide RNA” or “sgRNA”), and the crRNA equivalent portion of the guide RNA can be engineered to guide the Cas (e.g., Cas9) nuclease to target any desired sequence (see, e.g., Jinek et al. (2012) Science, 337:816-821; Jinek et al. (2013) eLife, 2: e00471; Segal (2013) eLife, 2: e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

The Cas nuclease can direct cleavage of one or both strands at a location in a target DNA sequence. For example, the Cas nuclease can be a nickase having one or more inactivated catalytic domains that cleaves a single strand of a target DNA sequence.

In some embodiments, the Cas nuclease is replaced with a different nuclease or nuclease system, such as TALE, Zinc Fingers, and the like.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, fragments thereof, mutants thereof, derivatives thereof, and combinations thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015: 40 (1): 58-66). Type II Cas nucleases include Cas1, Cas2, Csn2, Cas9, and Cpf1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Furthermore, the amino acid sequence of Acidaminococcus sp. BV3L6 is set forth, e.g., in NBCI Ref. Seq. No. WP_021736722.1. Some CRISPR-related endonucleases that are useful in the present invention are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. multocida, Sutterella wadsworthensis, proteobacterium, Legionella pneumophila, Parasutterella excrementihominis, Wolinella succinogenes, and Francisella novicida.

“Cpf1” refers to an RNA-guided double-stranded DNA-binding nuclease protein that is a type II Cas nuclease. Wild-type Cpf1 contains a RuvC-like endonuclease domain similar to the RuvC domain of Cas9, but does not have an HNH endonuclease domain and the N-terminal region of Cpf1 does not have the alpha-helix recognition lobe possessed by Cas9. The wild-type protein requires a single RNA molecule, as no tracrRNA is necessary. Wild-type Cpf1 creates staggered-end cuts and utilizes a T-rich protospacer-adjacent motif (PAM) that is 5′ of the guide RNA targeting sequence. Cpf1 enzymes have been isolated, for example, from Acidaminococcus and Lachnospiraceae.

“Cas9” refers to an RNA-guided double-stranded DNA-binding nuclease protein or nickase protein that is a type II Cas nuclease. Wild-type Cas9 nuclease has two functional domains, e.g., RuvC and HNH, that cut different DNA strands. The wild-type enzyme requires two RNA molecules (e.g., a crRNA and a tracrRNA), or alternatively, a single fusion molecule (e.g., a gRNA comprising a crRNA and a tracrRNA). Wild-type Cas9 utilizes a G-rich protospacer-adjacent motif (PAM) that is 3′ of the guide RNA targeting sequence and creates double-strand cuts having blunt ends. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

Variants of the Cas9 nuclease can include a single inactive catalytic domain, such as a RuvC.sup.- or HNH.sup.-enzyme or a nickase. A Cas9 nickase has only one active functional domain and can cut only one strand of the target DNA, thereby creating a single-strand break or nick. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of insertion/deletion (“indel”) mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the host cell or host organism. In some embodiments, the Cas9 protein is fused or linked directly to the RT. In some embodiments, the Cas9 protein is catalytically inactive. In some embodiments, the Cas9 protein is a mutant Cas9 that is a nickase. In some embodiments, the N-terminus of the Cas9 protein is linked or conjugated to the C-terminus of the RT. In some embodiments, the C-terminus of the Cas9 protein is linked or conjugated to the N-terminus of the RT. Nucleic acid molecules encoding the fusion protein can be prepare or synthesized by one of skill in the art to encode for such fusion proteins. In some embodiments, the Cas9 protein and the RT are not fused or linked to one another and are expressed from the same vector or different vectors. In some embodiments, the Cas9 protein is not fused or linked directly to the RT. In some embodiments, the Cas9 protein and the RT are expressed as separate molecules. In some embodiments, Cas9 protein and the RT are expressed from the same vector. In some embodiments, Cas9 protein and the RT are expressed from different vectors. In some embodiments, the Cas9, the RT, the msr/msd locus, and the gRNA are not linked or fused to each other. In some embodiments, the Cas9, the RT, the msr/msd locus, and the gRNA are each expressed as separate molecules.

In some embodiments, the Cas9 protein comprises an amino acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 69. In some embodiments, the Cas9 protein comprises the amino acid sequence of SEQ ID NO: 69.

In some embodiments, the CAS-RT fusion protein is also fused or linked to a bacterial single-stranded annealing protein (SSAP). In some embodiments, the CAS-RT fusion is linked to a protein domain that can stimulate increases HDR. Examples of such domains are RAD51, RAD18, and the like.

In some embodiments, the CAS-RT fusion protein is not fused or linked to a bacterial single-stranded annealing protein (SSAP). In some embodiments, the CAS, RT, and SSAP are not linked or fused to each other. In some embodiments, the SSAP is linked to the RT, but the construct does not comprise a CAS protein. In some embodiments, the N-terminus of the SSAP is linked or fused to the C-terminus of the RT. In some embodiments, the C-terminus of the SSAP is linked or fused to the N-terminus of the RT.

One non-limiting example of a SSAP is Bacteriophage T7 gp2.5 (gp.25), which has an amino acid sequence of

(SEQ ID NO: 83)
MAKKIFTSALGTAEPYAYIAKPDYGNEERGFGNPRGVYKVDLTIPNKD
PRCQRMVDEIVKCHEEAYAAAVEEYEANPPAVARGKKPLKPYEGDMPF
FDNGDGTTTFKFKCYASFQDKKTKETKHINLVVVDSKGKKMEDVPIIG
GGSKLKVKYSLVPYKWNTAVGASVKLQLESVMLVELATFGGGEDDWAD
EVEENGYVASGSAKASKPRDEESWDEDDEESEEADEDG.

The Bacteriophage T7 gp2.5 SSAP can be encoded by any nucleic acid sequence due to the degeneracy of the genetic code. However, a non-limiting example of a nucleic acid encoding Bacteriophage T7 gp2.5 SSAP is:

(SEQ ID NO: 84)
ATGGCCAAAAAGATCTTCACATCCGCTCTGGGCACAGCCGAGCCTTAC
GCCTACATCGCCAAGCCAGACTACGGCAACGAGGAACGGGGCTTCGGA
AATCCCAGAGGTGTGTACAAGGTGGACCTGACCATCCCCAACAAGGAC
CCCAGATGTCAGAGAATGGTGGATGAAATCGTGAAGTGCCACGAGGAG
GCCTACGCCGCTGCTGTTGAAGAGTACGAGGCTAATCCTCCAGCCGTG
GCCAGAGGCAAAAAACCTCTGAAACCTTACGAGGGCGATATGCCTTTC
TTCGACAACGGCGACGGCACCACCACCTTTAAGTTCAAGTGCTACGCC
AGTTTTCAGGACAAGAAGACCAAGGAAACAAAGCACATCAACCTGGTC
GTGGTGGACAGCAAGGGCAAGAAGATGGAAGATGTCCCCATTATCGGC
GGCGGCTCTAAACTGAAGGTGAAATACAGCCTGGTGCCTTATAAGTGG
AACACCGCCGTGGGCGCCAGCGTGAAGCTCCAGCTGGAATCCGTGATG
CTGGTGGAGCTGGCCACATTCGGCGGCGGCGAGGACGACTGGGCCGAC
GAGGTGGAAGAAAACGGCTACGTGGCCAGCGGCAGCGCCAAGGCCTCT
AAGCCTCGGGACGAAGAGAGCTGGGACGAGGACGATGAGGAAAGCGAG
GAAGCTGATGAGGATGGAGA.

Another example of a SSAP is: Escherichia coli Rac prophage RecT

(SEQ ID NO: 85)
MTKQPPIAKADLQKTQGNRAPAAIKNNDVISFINQPSMKEQLAAALPR
HMTAERMIRIATTEIRKVPALGNCDTMSFVSAIVQCSQLGLEPGSALG
HAYLLPFGNKNEKSGKKNVQLIIGYRGMIDLARRSGQIASLSARVVRE
GDEFNFEFGLDEKLIHRPGENEDAPVTHVYAVARLKDGGTQFEVMTRK
QIELVRSQSKAGNNGPWVTHWEEMAKKTAIRRLFKYLPVSIEIQRAVS
MDEKEPLTIDPADSSVLTGEYSVIDNSEE.

The Escherichia coli Rac prophage RecT SSAP can be encoded by any nucleic acid sequence due to the degeneracy of the genetic code. However, a non-limiting example of a nucleic acid encoding Escherichia coli Rac prophage RecT SSAP is:

(SEQ ID NO: 86)
ATGACCAAGCAGCCCCCCATCGCTAAGGCCGATCTGCAGAAGACACAA
GGCAACCGGGCCCCTGCTGCCATCAAGAACAACGACGTGATCAGCTTC
ATCAACCAGCCTTCTATGAAAGAACAGCTGGCCGCCGCTCTGCCCAGA
CACATGACAGCCGAGCGGATGATCAGAATCGCCACCACCGAGATCAGG
AAGGTGCCAGCCCTGGGCAATTGCGACACCATGTCTTTTGTGTCCGCA
ATCGTGCAATGTAGCCAGCTGGGCCTCGAGCCTGGCAGTGCTCTTGGC
CACGCCTATCTGCTGCCTTTTGGCAACAAGAATGAGAAAAGCGGAAAG
AAGAATGTGCAGCTGATCATCGGCTACAGAGGAATGATCGACCTGGCC
AGAAGAAGCGGCCAGATCGCCTCTCTGAGCGCTAGAGTGGTGCGGGAA
GGCGACGAGTTCAACTTCGAGTTCGGCCTGGATGAAAAGCTGATCCAC
AGACCTGGCGAAAACGAGGACGCCCCTGTGACCCACGTGTACGCCGTG
GCCAGACTGAAGGACGGCGGAACCCAGTTCGAGGTCATGACCAGAAAA
CAGATTGAGCTGGTGCGGTCTCAGTCAAAGGCCGGCAACAACGGCCCT
TGGGTCACACATTGGGAGGAAATGGCCAAGAAAACCGCCATCCGGAGA
CTGTTCAAGTACCTGCCTGTTAGCATCGAGATCCAGAGAGCCGTGTCC
ATGGACGAAAAGGAACCCCTGACCATCGATCCCGCCGACAGCTCCGTG
CTGACCGGCGAGTACAGCGTGATTGATAACAGCGAGGAA.

Another example of a SSAP is Escherichia phage lambda Bet (Lbet), which can have the amino acid sequence of:

(SEQ ID NO: 87)
MSTALATLAGKLAERVGMDSVDPQELITTLRQTAFKGDASDAQFIALL
IVANQYGLNPWTKEIYAFPDKQNGIVPVVGVDGWSRIINENQQFDGMD
FEQDNESCTCRIYRKDRNHPICVTEWMDECRREPFKTREGREITGPWQ
SHPKRMLRHKAMIQCARLAFGFAGIYDKDEAERIVENTAYTAERQPER
DITPVNDETMQEINTLLIALDKTWDDDLLPLCSQIFRRDIRASSELTQ
AEAVKALGFLKQKAAEQKVAA.

The Escherichia phage lambda Bet (Lbet) SSAP can be encoded by any nucleic acid sequence due to the degeneracy of the genetic code. However, a non-limiting example of a nucleic acid encoding Escherichia phage lambda Bet (Lbet) SSAP is:

(SEQ ID NO: 88)
ATGTCTACAGCCCTGGCCACCCTGGCTGGCAAGCTGGCCGAGAGAGTG
GGAATGGACAGCGTGGACCCCCAGGAGCTGATCACCACCCTGCGGCAG
ACCGCCTTCAAGGGCGACGCCAGCGACGCCCAGTTTATCGCCCTGCTG
ATCGTCGCTAATCAATACGGCCTGAACCCCTGGACCAAGGAAATCTAT
GCCTTCCCCGACAAACAGAACGGCATCGTGCCTGTGGTGGGCGTTGAC
GGCTGGTCCAGAATCATCAATGAGAACCAGCAGTTCGACGGAATGGAT
TTCGAGCAAGATAACGAAAGCTGTACATGCAGAATCTACAGAAAGGAC
AGAAACCACCCTATCTGCGTGACCGAGTGGATGGACGAATGTAGGCGG
GAACCTTTCAAAACCAGAGAGGGCAGAGAAATTACCGGCCCATGGCAG
AGCCACCCCAAGCGGATGCTGAGACACAAGGCCATGATCCAGTGCGCC
AGACTGGCCTTTGGCTTCGCTGGCATCTACGACAAGGATGAGGCCGAG
CGGATCGTGGAAAACACCGCCTACACCGCTGAACGGCAACCTGAGCGC
GACATCACACCTGTGAACGACGAGACAATGCAGGAGATTAACACACTG
CTCATCGCTCTGGACAAGACCTGGGATGACGATCTGCTGCCTCTGTGC
AGCCAGATCTTCAGAAGAGATATCCGGGCCAGCAGCGAACTGACACAG
GCCGAAGCCGTGAAGGCCCTGGGCTTCCTGAAGCAGAAAGCCGCCGAG
CAGAAGGTGGCCGCT.

For genome editing methods, the Cas nuclease can be a Cas9 fusion protein such as a polypeptide comprising the catalytic domain of a restriction enzyme (e.g., FokI) linked to dCas9. The FokI-dCas9 fusion protein (fCas9) can use two guide RNAs to bind to a single strand of target DNA to generate a double-strand break.

In some embodiments, the Cas-RT fusion comprises a linker between the Cas protein and the RT protein. In some embodiments, the fusion protein comprises one or more NLS (nuclear localization sequences). In some embodiments, the fusion protein comprises a polypeptide having the formula of: C₁-L₁-R₁, wherein C₁ is a Cas, L₁ is a peptide linker and R₁ is a RT protein. In some embodiments, the Cas is as provided for herein. In some embodiments, the Cas can be replaced with the SSAP protein to create a SSAP-RT fusion protein, such as provided for herein. In some embodiments, the linker a glycine/serine or glycine/alanine linker, or any combination thereof. In some embodiments, the linker is a XTEN linker. An example of an XTEN linker is an amino acid sequence of SGGSSGGSSGSETPGTSESATPESSGGSSGGSS (SEQ ID NO: 89). In some embodiments, the linker comprises a sequence of GGGGSGGGGSGGGGSGGGGS (SEQ ID NO: 90) or GGGGSGGGGSGGGGS (SEQ ID NO: 91). This is simply a non-limiting example and the linker can have varying number of GGGGS (SEQ ID NO: 92) or GGGGA (SEQ ID NO: 93) repeats. In some embodiments, the linker comprises 1, 2, 3, 4, 5, 6, 7, 8, 9, or 10 of the GGGGS (SEQ ID NO: 92) or GGGGA (SEQ ID NO: 93) repeats.

In some embodiments, the fusion protein is in a configuration as illustrated in FIG. 3, FIG. 4, or FIG. 11A. The configurations are illustrated in the N→C orientation from left to right, but the domains could also be in the opposite orientation. In some embodiments, the fusion protein is in a configuration as illustrated in FIG. 11A. The configurations are illustrated in the N→C orientation from left to right, but the domains could also be in the opposite orientation.

In some embodiments, the polypeptide comprises a first and/or second NLS sequence. In some embodiments, the NLS sequence is a SV40 NLS sequence (e.g., PKKKRKV (SEQ ID NO: 94)). In some embodiments, the NLS sequence is a Nuceloplasmin NLS sequence (e.g., KRPAATKKAGQAKKKK (SEQ ID NO: 95)). In some embodiments, the NLS sequences is a C-myc NLS sequence (PAAKRVKLD (SEQ ID NO: 96)) In some embodiments, the first NLS is to the N-terminus of C₁ and the second NLS is to the C-terminus of R₁. In some embodiments, the fusion protein comprises a C-Myc NLS (and a sv40 NLS. In some embodiments, the fusion protein comprises a C-Myc NLS, a sv40 NLS, and a Nuceloplasmin NLS sequence. Examples of such configurations are illustrated in FIG. 11A.

In some embodiments, a polypeptide, or nucleic acid molecule encoding the same, is provided having the formula of: of (N₁)_{q}-C₁-L₁-(N₂)_{qq}-R₁-(N₃)_{qqq}, wherein C1 is a nuclease, such as Cas (e.g., CAS9), L1 is a peptide linker, R1 is a RT protein, and N1 and N2 are each, independently, a NLS sequence, wherein q, qq, or qqq, are each, independently, 0, 1, 2, or 3. In some embodiments, q is 1 or 2. In some embodiments, when q is 2, the NLS sequences, which can be the same or different, can be separated by a linker sequence. In some embodiments, qqq is 1 or 2. In some embodiments, when qqq is 2, the NLS sequences, which can be the same or different, can be separated by a linker sequence. In some embodiments, qq is 0 or 1. In some embodiments, when qq is 1, the NLS can comprise a linker sequence at the C-terminal end of the sequence and N-terminal to R₁.

In some embodiments, polypeptides, or nucleic acid molecules encoding the same, are provided having the formula of:

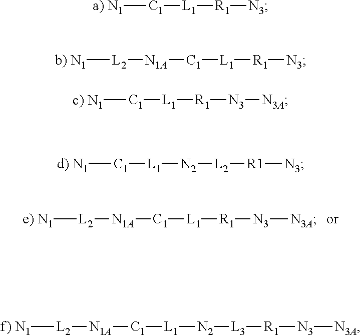

wherein:
   each N1, N1A, N2, N3, or N3A is, independently, a nuclear localization sequence (NLS);
   each L1, L2, or L3 is independently, a linker amino acid sequence; and
wherein each NLS can be the same or different.

As provided for herein, wherein N₁, N_{1A}, N₂, N₃, or N_{3A}, can each comprise, independently a SV40 NLS sequence, a cMyc NLS sequence, or a Nuceloplasmin NLS sequence. In some embodiments, N₁ or N_{1A} comprises a cMyc or SV40 NLS sequence. In some embodiments, N₂ comprises a SV40 NLS sequence or a Nuceloplasmin NLS sequence. In some embodiments, N₃ or N_{3A} comprises a Nuceloplasmin NLS sequence. In some embodiments, the polypeptide has a formula as depicted in the polypeptides illustrated in FIG. 11A. In some embodiments, L1, L2, or L3, or the linker are each, independently, an XTEN linker, a G/S linker, or a G/A linker, or any combination thereof. In some embodiments, R1 comprises a RT sequence Ec86, Ec48, Ec73, Ec107, or Sa163.

In some embodiments, the polypeptide has an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74. In some embodiments, the polypeptide has an amino acid sequence of any one of SEQ ID NO: 70, 71, 72, 73, or 74. In some embodiments, the polypeptide has an amino acid sequence of SEQ ID NO: 70. In some embodiments, the polypeptide has an amino acid sequence of SEQ ID NO: 71. In some embodiments, the polypeptide has an amino acid sequence of SEQ ID NO: 72. In some embodiments, the polypeptide has an amino acid sequence of SEQ ID NO: 73. In some embodiments, the polypeptide has an amino acid sequence of SEQ ID NO: 74.

In some embodiments, the variant polypeptide comprises a domain that has RT activity and/or nuclease activity. In some embodiments, the variant polypeptide has at least 80%, at least 85%, at least 90%, at least 91%, at least 92%, at least 93%, at least 94%, at least 95%, at least 96%, at least 97%, at least 98%, or at least 99% identity to the sequences recited therein provided that the polypeptide has 1, 2, 3, or 4 NLS amino acid sequences. In some embodiments, the variant polypeptide has at least 80%, at least 85%, at least 90%, at least 91%, at least 92%, at least 93%, at least 94%, at least 95%, at least 96%, at least 97%, at least 98%, or at least 99% identity to the sequences recited therein provided that the polypeptide comprises a RT enzyme, which is catalytically active. In some embodiments, the variant polypeptide has at least 80%, at least 85%, at least 90%, at least 91%, at least 92%, at least 93%, at least 94%, at least 95%, at least 96%, at least 97%, at least 98%, or at least 99% identity to the sequences recited therein provided that the polypeptide comprises a nuclease, such as a nuclease provided for herein.

In some embodiments, the fusion protein comprises a heterologous tag, which can be used for detection and/or purification of the fusion protein. In some embodiments, the tag is a polyhistidine tag. In some embodiments, the tag is a FLAG tag.

In some embodiments, the fusion protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of:

(SEQ ID NO: 97)
MAPKKKRKVGIHGVPAADKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPGTSESATPESSGGSSGGSSKSA
EYLNTFRLRNLGLPVMNNLHDMSKATRISVETLRLLIYTADFRYRIYTVEKKGPEKRMRTIYQPSRELKAL
QGWVLRNILDKLSSSPFSIGFEKHQSILNNATPHIGANFILNIDLEDFFPSLTANKVFGVFHSLGYNRLIS
SVLTKICCYKNLLPQGAPSSPKLANLICSKLDYRIQGYAGSRGLIYTRYADDLTLSAQSMKKVVKARDFLF
SIIPSEGLVINSKKTCISGPRSQRKVTGLVISQEKVGIGREKYKEIRAKIHHIFCGKSSEIEHVRGWLSFI
LSVDSKSHRRLITYISKLEKKYGKNPLNKAKTKRPAATKKAGQAKKKK;
(SEQ ID NO: 98)
MAPKKKRKVGIHGVPAADKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPGTSESATPESSGGSSGGSSGRP
YVTLNLNGMFMDKFKPYSKSNAPITTLEKLSKALSISVEELKAIAELPLDEKYTLKEIPKIDGSKRIVYSL
HPKMRLLQSRINKRIFKELVVFPSFLFGSVPSKNDVLNSNVKRDYVSCAKAHCGAKTVLKVDISNFFDNIH
RDLVRSVFEEILHIKDEALEYLVDICTKDDFVVQGALTSSYIATLCLFAVEGDVVRRAQRKGLVYTRLVDD
ITVSSKISNYDFSQMQSHIERMLSEHDLPINKRKTKIFHCSSEPIKVHGLRVDYDSPRLPSDEVKRIRASI
HNLKLLAAKNNTKTSVAYRKEFNRCMGRVNKLGRVAHEKYESFKKQLQAIKPMPSKRDVAVIDAAIKSLEL
SYSKGNQNKHWYKRKYDLTRYKMIILTRSESFKEKLECFKSRLASLKPKRPAATKKAGQAKKKK;
(SEQ ID NO: 99)
MAPKKKRKVGIHGVPAADKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPGTSESATPESSGGSSGGSSRIY
SLIDSQTLMTKGFASEVMRSPEPPKKWDIAKKKGGMRTIYHPSSKVKLIQYWLMNNVFSKLPMHNAAYAFV
KNRSIKSNALLHAESKNKYYVKIDLKDFFPSIKFTDFEYAFTRYRDRIEFTTEYDKELLQLIKTICFISDS
TLPIGFPTSPLIANFVARELDEKLTQKLNAIDKLNATYTRYADDIIVSTNMKGASKLILDCFKRTMKEIGP
DFKINIKKFKICSASGGSIVVTGLKVCHDFHITLHRSMKDKIRLHLSLLSKGILKDEDHNKLSGYIAYAKD
IDPHFYTKLNRKYFQEIKWIQNLHNKVEKRPAATKKAGQAKKKK;
(SEQ ID NO: 100)
MAPKKKRKVGIHGVPAADKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPGTSESATPESSGGSSGGSSDAT
RTTLLALDLFGSPGWSADKEIQRLHALSNHAGRHYRRIILSKRHGGQRLVLAPDYLLKTVQRNILKNVLSQ
FPLSPFATAYRPGCPIVSNAQPHCQQPQILKLDIENFFDSISWLQVWRVFRQAQLPRNVVTMLTWICCYND
ALPQGAPTSPAISNLVMRRFDERIGEWCQARGITYTRYCDDMTFSGHFNARQVKNKVCGLLAELGLSLNKR
KGCLIAACKRQQVTGIVVNHKPQLAREARRALRQEVHLCQKYGVISHLSHRGELDPSGDLHAQATAYLYAL
QGRINWLLQINPEDEAFQQARESVKRMLVAWKRPAATKKAGQAKKKK;
OR
(SEQ ID NO: 101)
MAPKKKRKVGIHGVPAADKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPGTSESATPESSGGSSGGSSTAK
LESHVPAAPPVSAEAPAPTRPDAAKQEARRAHHEALRLRWKAIEEAGGTDAWVRQQLVAKGVAAEEVDFES
LSDKQKAAWKEKKKAEATERRAQKRLAWEAWKATHIHHLGVGVHWDEAGGPDKEDVAGREERAKANGLPEG
LDSVEALAKALGISVSRLRWFSFHREVDTGTHYQTWEIPKRDGGKRTLTAPKRELKAVQRWVLANVVERLP
VHGAAHGFVAGRSILTNALAHQGADVVVKVDMKDFFPSVTWPRVKGLLRKGGLPENLATLLALLSTEAPRE
VVRFRGETLYVAKGPRALPQGAPTSPALTNALCLRLDKRLSALSKRLGFTYTRYADDLTFSWRRAKKSRQK
ELPLADAPVALLLARVKGVLEAEGFTLHPDKTRVQRKGSRQRVTGLVVNEAPEGVPGARVPRDVVRRLRAA
IHNREQGKPGPTGETLEQLKGLAAFLHMTDAEKGRAFLRRLEALEKRQTAKRPAATKKAGQAKKKK.

In some embodiments, the fusion protein comprises an amino acid sequence of any one of SEQ ID NO: 97, 98, 99, 100, or 101. In some embodiments, the fusion protein comprises an amino acid sequence of any one of SEQ ID NO: 97. In some embodiments, the fusion protein comprises an amino acid sequence of any one of SEQ ID NO: 98. In some embodiments, the fusion protein comprises an amino acid sequence of any one of SEQ ID NO: 99. In some embodiments, the fusion protein comprises an amino acid sequence of any one of SEQ ID NO: 100. In some embodiments, the fusion protein comprises an amino acid sequence of any one of SEQ ID NO: 101.

In some embodiments, a polynucleotide molecule, such as an isolated polynucleotide molecule is provided. In some embodiments, the molecule comprises a msr locus and a msd locus with a donor DNA sequence inserted therein, wherein at least one nucleotide is deleted or mutated as compared to the wild-type msr and msd locus.

In some embodiments, the wild-type msr locus and the msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity of any one of SEQ ID NO: 1, or 2. In some embodiments, the wild-type msr locus and the msd locus comprises the sequence of any one of SEQ ID NO: 1, or 2. In some embodiments, the wild-type msr locus and the msd locus comprises the sequence of SEQ ID NO: 1. In some embodiments, the wild-type msr locus and the msd locus comprises the sequence of SEQ ID NO: 2.

In some embodiments, the mutated msr locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to the wild-type.

In some embodiments, the polynucleotide comprises a nucleic acid molecule comprising a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18 upstream of the donor DNA nucleic acid sequence and a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23 downstream of the donor DNA nucleic acid sequence.

In some embodiments, the polynucleotide comprises a nucleic acid molecule comprising a nucleic acid sequence selected from any one of SEQ ID NO: 4, 5, 6, 7, 8, 14, 15, 16, 17, or 18 upstream of the donor DNA nucleic acid sequence and a nucleic acid sequence selected from any one of SEQ ID NO: 9, 10, 11, 12, 13, 19, 20, 21, 22, or 23 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 4 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 5 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 6 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 7 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 8 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 14 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 15 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 16 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 17 upstream of the donor DNA nucleic acid sequence and a nucleic acid sequence of SEQ ID NO: 23 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 9 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 10 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 11 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 12 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 13 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 19 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 20 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 21 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 22 downstream of the donor DNA nucleic acid sequence. In some embodiments, the polynucleotide comprises a nucleic acid molecule comprising a nucleic acid sequence of SEQ ID NO: 18 upstream of the donor DNA nucleic acid sequence and a nucleic acid sequence of SEQ ID NO: 23 downstream of the donor DNA nucleic acid sequence.

In some embodiments, the polynucleotide comprises a nucleic acid molecule comprising a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 49, 50, 51, 52, 53, 54, 55, 56, 57, or 58 upstream of the donor DNA nucleic acid sequence and a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 509, 60, 61, 62, 63, 64, 65, 66, 67, or 68 downstream of the donor DNA nucleic acid sequence.

In some embodiments, the polynucleotide comprises a nucleic acid molecule comprising a nucleic acid sequence selected from any one of SEQ ID NO: 49, 5, 6, 7, 8, 14, 15, 16, 17, or 18 upstream of the donor DNA nucleic acid sequence and a nucleic acid sequence selected from any one of SEQ ID NO: 59, 10, 11, 12, 13, 19, 20, 21, 22, or 23 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 49 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 50 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 51 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 52 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 53 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 54 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 55 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 56 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 57 upstream of the donor DNA nucleic acid sequence and a nucleic acid sequence of SEQ ID NO: 68 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 59 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 60 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 61 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 62 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 63 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 64 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 65 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 66 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 67 downstream of the donor DNA nucleic acid sequence. In some embodiments, the polynucleotide comprises a nucleic acid molecule comprising a nucleic acid sequence of SEQ ID NO: 58 upstream of the donor DNA nucleic acid sequence and a nucleic acid sequence of SEQ ID NO: 68 downstream of the donor DNA nucleic acid sequence.

In some embodiments, the polynucleotide comprises a nucleic acid molecule having a formula of 5′-M1-X1-M2-3′, wherein M1 is a fragment of the msr/msd locus, X1 is the donor DNA sequence; and M2 is a fragment of the msd/msr locus. In some embodiments, M1 comprises a nucleic acid sequence of any one of SEQ ID NO: 24, 25, 26, 27, 28, 29, 30, 31, 32, or 33, or a fragment of any of the foregoing or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any of the foregoing.

In some embodiments, M2 comprises a nucleic acid sequence of any one of SEQ ID NO: 34, 35, 36, 37, 38, 39, 40, 41, 42, or 43, or a fragment of any of the foregoing or a sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any of the foregoing.

In some embodiments, the nucleic acid molecule comprises an M1 having a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and an M2 having a nucleic acid sequence having at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43. In some embodiments, the nucleic acid molecule comprises an M1 having a nucleic acid sequence of any one of SEQ ID NO: 24, 25, 26, 27, 28, 29, 30, 31, 32, or 33; and an M2 having a nucleic acid sequence of any one of SEQ ID NO: 34, 35, 36, 37, 38, 39, 40, 41, 42, or 43.

Although the polynucleotides provided for herein are represented by DNA nucleotides, the equivalent transcribed RNA sequences are also provided.

The polynucleotide sequences can be present in a vector, cell, liposome or other composition, including, but not limited to, a pharmaceutical composition.

In some embodiments, a composition is provided that contains a retron msd without a gRNA sequence. The retron msd or msd/msr is co-expressed in a cell with a SSAP, which enables integration of the retron msd into the genetic locus. This can be done without a Cas protein, such as CAS9 or even an active form of CAS9.

In some embodiments, a nucleotide sequence encoding the Cas nuclease or other protein, such as the SSAP, is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Depending on the host cell and expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Promoters may also be inducible (i.e., capable of responding to environmental factors and/or external stimuli that can be artificially controlled). Suitable promoters include, but are not limited to: RNA polymerase II promoters (e.g., pGAL7 and pTEF1), RNA polymerase III promoters (e.g., RPR-tetO, SNR52, and tRNA-tyr), the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc. Suitable terminators include, but are not limited to SNR52 and RPR terminator sequences (non-limiting examples of which are set forth in SEQ ID NO:37 and 38, respectively), which can be used with transcripts created under the control of a RNA polymerase III promoter. Additionally, various primer binding sites may be incorporated into a vector to facilitate vector cloning, sequencing, genotyping, and the like. As a non-limiting example, the Pci1-Up sequence set forth in SEQ ID NO:26 can be incorporated. Other suitable promoter, enhancer, terminator, and primer binding sequences will readily be known to one of skill in the art.

Methods for introducing polypeptides and nucleic acids into a host cell are known in the art, and any known method can be used to introduce a nuclease or a nucleic acid (e.g., a nucleotide sequence encoding the nuclease or RT, a DNA-targeting RNA (e.g., a guide RNA), a donor repair template for homology-directed repair (HDR), etc.) into a cell. Non-limiting examples of suitable methods include electroporation, viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments, the components of the CRISPR-retron system can be introduced into a cell using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct), a RT component, and one or more nucleic acid components such as a DNA-targeting RNA (e.g., a guide RNA) and/or a donor repair template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

In some embodiments, cells are provided that have been transformed by vectors and constructs provided for herein. The compositions and methods provided for herein can be used for genome editing of any host cell of interest. The host cell can be a cell from any organism, e.g., human cell, a bacterial cell, an archaeal cell, a cell of a single-cell eukaryotic organism, a plant cell (e.g., a rice cell, a wheat cell, a tomato cell, an Arabidopsis thaliana cell, a Zea mays cell and the like), an algal cell (e.g., Botryococcus braunii, Chlamydomonas reinhardtii, Nannochloropsis gaditana, Chlorella pyrenoidosa, Sargassum patens C. Agardh, and the like), a fungal cell (e.g., yeast cell, etc.), an animal cell, a cell from an invertebrate animal (e.g., fruit fly, cnidarian, echinoderm, nematode, etc.), a cell from a vertebrate animal (e.g., fish, amphibian, reptile, bird, mammal, etc.), a cell from a mammal, a cell from a human, a cell from a healthy human, a cell from a human patient, a cell from a cancer patient, etc. In some embodiments, the cell that is edited is taken from one subject and administered (transplanted) into another subject or patient. Thus, the edited cells can be used as an autologous or allogeneic therapy.

The cell can be any type of such. For example, the cell can be, but not limited to, a stem cell, e.g., embryonic stem cell, induced pluripotent stem cell, adult stem cell, e.g., mesenchymal stem cell, neural stem cell, hematopoietic stem cell, organ stem cell, a progenitor cell, a somatic cell, e.g., fibroblast, hepatocyte, heart cell, liver cell, pancreatic cell, muscle cell, skin cell, blood cell, neural cell, immune cell, and any other cell of the body, e.g., human body. The cells can be primary cells or primary cell cultures derived from a subject, e.g., an animal subject or a human subject, and allowed to grow in vitro for a limited number of passages. In some embodiments, the cells are disease cells or derived from a subject with a disease. For instance, the cells can be cancer or tumor cells. The cells can also be immortalized cells (e.g., cell lines), for instance, from a cancer cell line.

Cells can be harvested from a subject by any standard method. For instance, cells from tissues, such as skin, muscle, bone marrow, spleen, liver, kidney, pancreas, lung, intestine, stomach, etc., can be harvested by a tissue biopsy or a fine needle aspirate. Blood cells and/or immune cells can be isolated from whole blood, plasma or serum. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be skewed towards particular populations and phenotypes. For example, the T cells can be skewed to phenotypically comprise, CD45RO(−), CCR7(+), CD45RA(+), CD62L(+), CD27(+), CD28(+) and/or IL-7Ra(+). Suitable cells can be selected that comprise one of more markers selected from a list comprising: CD45RO(−), CCR7(+), CD45RA(+), CD62L(+), CD27(+), CD28(+) and/or IL-7Ra(+). Induced pluripotent stem cells can be generated from differentiated cells according to standard protocols described in, for example, U.S. Pat. Nos. 7,682,828, 8,058,065, 8,530,238, 8,871,504, 8,900,871 and 8,791,248, the disclosures are herein incorporated by reference in their entirety for all purposes.

In some embodiments, the cell that is edited is edited in vivo, ex vivo, or in vitro.

In some embodiments, methods for modifying one or more target nucleic acids of interest at one or more target loci within a genome of a host cell are provided. In some embodiments, the method comprises: transforming or transducing the host cell with a construct as provided for herein; and (b) culturing the cell or progeny thereof under conditions sufficient for expressing from the construct a retron donor DNA-guide molecule comprising a retron transcript and a guide RNA (gRNA) molecule. The retron can self-prime reverse transcription by a RT expressed by the host cell or the transformed progeny of the cell. In some embodiments, the retron transcript is reverse transcribed to produce a multicopy single-stranded DNA (msDNA) molecule having one or more donor DNA sequences, wherein the one or more donor DNA sequences are homologous to the one or more target loci and comprise sequence modifications compared to the one or more target nucleic acids. In some embodiments, the one or more target loci are cut by a nuclease expressed by the host cell or the transformed progeny of the host cell, wherein the site of nuclease cutting is specified by the gRNA. However, in some embodiments, the target loci is not cut by a nuclease heterologously expressed. In some embodiments, the nuclease is a catalytically in active nuclease, such as dCAS. In some embodiments, the one or more donor DNA sequences recombine with the one or more target nucleic acid sequences to insert, delete, and/or substitute one or more bases of the sequence of the one or more target nucleic acid sequences to induce one or more sequence modifications at the one or more target loci within the genome. In some embodiments, an entire gene is replaced. In some embodiments, one or more exons of a gene is replaced.

In some embodiments, the host cell is capable of expressing the RT prior to transforming the host cell with the vector. In some instances, the RT is encoded in a sequence that is integrated into the genome of the host cell. In some embodiments, the RT is encoded in a sequence on a separate plasmid or vector from the retron construct. In some embodiments, the host cell is capable of expressing the RT at the same time as, or after, transforming the host cell with the vector. In some instances, the RT is expressed from the vector. In other instances, the RT is encoded in a sequence on a separate plasmid or vector from the retron.

In some embodiments, the host cell is capable of expressing the nuclease (e.g., Cas9) prior to transforming the host cell with the vector or construct. In some embodiments, the nuclease is encoded in a sequence that is integrated into the genome of the host cell. In other instances, the nuclease is encoded in a sequence on a separate plasmid. In other embodiments, the host cell is capable of expressing the nuclease at the same time as, or after, transforming the host cell with the vector. In some instances, the nuclease is expressed from the vector. In other instances, the nuclease is encoded in a sequence on a separate plasmid.

In some embodiments, the vector comprises a retron-gRNA cassette that, when transcribed, yields a retron transcript and gRNA that are physically coupled. In such embodiments, the resulting donor DNA sequence within the msDNA and the gRNA can also be physically coupled. In particular embodiments, the retron transcript and gRNA subsequently become physically uncoupled (e.g., before or after reverse transcription of the retron transcript occurs). Physical uncoupling of the retron transcript and the gRNA can result from, for example, ribozyme cleavage (e.g., the retron-gRNA cassette also contains a ribozyme sequence). In such embodiments, the resulting donor DNA sequence within the msDNA and the gRNA will be physically uncoupled (e.g., during genome editing and/or screening).

In some embodiments, the retron transcript and the gRNA are not initially physically coupled. In some embodiments, the retron transcript and the gRNA are subsequently joined together. Transcription event(s) that result in the production of the retron transcript and/or gRNA can occur inside a host cell, outside of a host cell (e.g., followed by introduction of the retron transcript and/or gRNA into the host cell), or a combination thereof. In some embodiments, the one or more target nucleic acids of interest are modified by a donor DNA sequence (e.g., within a msDNA) and a gRNA that are never physically coupled. For example, the donor DNA sequence and the gRNA can be expressed from different cassettes (e.g., which are contained in the same vector or different vectors) and the donor DNA sequence and the gRNA can act in trans. In some embodiments, the gRNA is fused or linked to the 5′ end of the retron msrimsd molecule. In some embodiments, the gRNA is fused or linked to the 3′ end of the retron msr/msd molecule. In some embodiments, the gRNA is not linked to the retron msr/msd molecule.

To assess the efficiency and/or precision of genome editing (e.g., testing for whether an edit has been made and/or the accuracy of the edit), the target DNA can be analyzed by standard methods known to those in the art. For example, indel mutations can be identified by sequencing using the SURVEYOR® mutation detection kit (Integrated DNA Technologies, Coralville, Iowa) or the Guide-It™ Indel Identification Kit (Clontech, Mountain View, Calif.). Homology-directed repair (HDR) can be detected by PCR-based methods, and in combination with sequencing or RFLP analysis. Non-limiting examples of PCR-based kits include the Guide-it Mutation Detection Kit (Clontech) and the GeneArt® Genomic Cleavage Detection Kit (Life Technologies, Carlsbad, Calif.). Deep sequencing can also be used, particularly for a large number of samples or potential target/off-target sites.

In some embodiments, editing efficiency can be assessed by employing a reporter or selectable marker to examine the phenotype of an organism or a population of organisms. In some instances, the marker produces a visible phenotype, such as the color of an organism or population of organisms. As a non-limiting example, edits can be made that either restore or disrupt the function of metabolic pathways that confer a visible phenotype (e.g., a color) to the organism. In the scenario where a successful genome edit results in a color change in the target organism (e.g., because the edit disrupts a metabolic pathway that results in a color change or because the edit restores function in a pathway that results in a color change), the absolute number or the proportion of organisms or their progeny that exhibit a color change (e.g., an estimated or direct count of the number of organisms exhibiting a color change divided by the total number of organisms for which the genomes were potentially edited) can serve as a measure of editing efficiency. In some instances, the phenotype is examined by growing the target organisms and/or their progeny under conditions that result in a phenotype, wherein the phenotype may not be visible under ordinary growth conditions. As a non-limiting example, growing yeast in a culture medium that is adenine deficient can lead to a particular phenotype (e.g., a color change) in yeast cells that possess a genetic defect in adenine synthesis. As such, growing yeast cells in adenine-deficient media can allow one to discern the effect of genome edits that putatively target adenine biosynthesis loci.

In some embodiments, the reporter or selectable marker is a fluorescent tagged protein, an antibody, a labeled antibody, a chemical stain, a chemical indicator, or a combination thereof. In other embodiments, the reporter or selectable marker responds to a stimulus, a biochemical, or a change in environmental conditions. In some instances, the reporter or selectable marker responds to the concentration of a metabolic product, a protein product, a synthesized drug of interest, a cellular phenotype of interest, a cellular product of interest, or a combination thereof. A cellular product of interest can be, as a non-limiting example, an RNA molecule (e.g., messenger RNA (mRNA), long non-coding RNA (lncRNA), microRNA (miRNA)).

Editing efficiency can also be examined or expressed as a function of time. For example, an editing experiment can be allowed to run for a fixed period of time (e.g., 24 or 48 hours) and the number of successful editing events in that fixed time period can be determined. Alternatively, the proportion of successful editing events can be determined for a fixed period of time. Typically, longer editing periods will result in a larger number of successful editing events. Editing experiments or procedures can run for any length of time. In some embodiments, a genome editing experiment or procedure runs for several hours (e.g., about 1, 2, 3, 4, 5, 6, 7, 8, 9, 10, 11, 12, 13, 14, 15, 16, 17, 18, 19, 20, 21, 22, 23, or 24 hours). In other embodiments, a genome editing experiment or procedure runs for several days (e.g., about 1, 2, 3, 4, 5, 6, or 7 days).

In some embodiments, in addition to the length of time of the editing period, editing efficiency can be affected by the choice of gRNA, donor DNA sequence, the choice of promoter used, or a combination thereof.

In some embodiments, editing efficiency is compared to a control efficiency. In some embodiments, the control efficiency is determined by running a genome editing experiment in which the retron transcript and gRNA molecule are never physically coupled, or are initially coupled but subsequently become uncoupled. In some instances, the retron transcript and gRNA molecule are initially coupled and then become uncoupled (e.g., by ribozyme cleavage). In other instances, the retron-guide RNA (gRNA) cassette is configured such that the transcript products of the retron and gRNA coding region are never physically coupled. In yet other instances, the retron transcript and gRNA are introduced into the host cell separately. In some instances, the methods and compositions of the present invention result in at least about a 1.3- to 3-fold (i.e., at least about a 1.3-, 1.4-, 1.5-, 1.6-, 1.7-, 1.8-, 1.9-, 2-, 2.1-, 2.2-, 2.3-, 2.4-, 2.5-, 2.6-, 2.7-, 2.8-, 2.9-, or 3-fold) increase in efficiency, compared to when the retron transcript and gRNA are not physically coupled during editing. In other instances, at least about a 3- to 10-fold increase (i.e., at least about a 3-, 4-, 5-, 6-, 7-, 8-, 9-, or 10-fold) increase in efficiency is produced, compared to when the retron transcript and gRNA are not physically coupled during editing. In particular instances, at least about a 10- to 100-fold (i.e., at least about 10-, 20-, 30-, 40-, 50-, 60-, 70-, 80-, 90-, or 100-fold) increase in efficiency is produced, compared to when the retron transcript and gRNA are not physically coupled during editing.

In some embodiment, the methods are performed by performing editing experiments or procedures in a multiplex format. In some embodiments, multiplexing comprises cloning two or more editing retron-gRNA cassettes in tandem into a single vector. In some instances, at least about 10 retron-gRNA cassettes (i.e., at least about 2, 3, 4, 5, 6, 7, 8, 9, or 10 retron-gRNA cassettes) are cloned into a single vector.

In some embodiments, multiplexing comprises transforming a host cell with two or more vectors. Each vector can comprise one or multiple retron-gRNA cassettes. In some instances, at least about 10 vectors (i.e., at least about 2, 3, 4, 5, 6, 7, 8, 9, or 10 vectors) are used to transform an individual host cell. In some embodiments, the retron and gRNA can be expressed from separate vectors and thereby being separate transcripts. In some embodiments, the retron and gRNA are part of the same transcript.

In some embodiments, multiplexing comprises transforming two or more individual host cells, each with a different vector or combination of vectors. In some instances, at least about 2 host cells (i.e., at least about 2, 3, 4, 5, 6, 7, 8, 9, or 10 host cells) are transformed. In other instances, between about 10 and 100 host cells (i.e., about 10, 15, 20, 25, 30, 35, 40, 45, 50, 55, 60, 65, 70, 75, 80, 85, 90, 95, or 100 host cells) are transformed. In still other instances, between about 100 and 1,000 host cells (i.e., about 100, 200, 300, 400, 500, 600, 700, 800, 900, or 1,000 host cells) are transformed. In particular instances, between about 1,000 and 10,000 host cells (i.e., about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000 host cells are transformed). In some other instances, between about 10,000 and 100,000 host cells (i.e., about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 host cells) are transformed. In other instances, between about 100,000 and 1,000,000 host cells (i.e., at least about 100,000, 150,000, 200,000, 250,000, 300,000, 350,000, 400,000, 450,000, 500,000, 550,000, 600,000, 650,000, 700,000, 750,000, 800,000, 850,000, 900,000, 950,000 or 1,000,000 host cells) are transformed. In some instances, more than about 1,000,000 host cells are transformed. Also, multiple embodiments of multiplexing can be combined.

In some embodiments, by using one or a combination of the various multiplexing embodiments, it is possible to modify and/or screen any number of loci within a genome. In some instances, at least about 10 (i.e., about 1, 2, 3, 4, 5, 6, 7, 8, 9, or 10) genetic loci are modified or screened. In other instances, between about 10 and 100 (i.e., about 10, 15, 20, 25, 30, 35, 40, 45, 50, 55, 60, 65, 70, 75, 80, 85, 90, 95, or 100) loci are modified or screened. In still other instances, between about 100 and 1,000 genetic loci (i.e., about 100, 150, 200, 250, 300, 350, 400, 450, 500, 550, 600, 650, 700, 750, 800, 850, 900, 950, or 1,000 genetic loci) are modified or screened. In some other instances, between about 1,000 and 100,000 genetic loci (i.e., about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500 10,000, 15,00, 20,000, 25,000, 30,000, 35,000, 40,000, 45,000, 50,000, 55,000, 60,000, 65,000, 70,000, 75,000, 80,000, 85,000, 90,000, 95,000, or 100,000 genetic loci) are modified or screened. In particular instances, between about 100,000 and 1,000,000 genetic loci (i.e., about 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000 genetic loci) are modified or screened. In certain instances, more than about 1,000,000 loci are screened.

In some embodiments, pharmaceutical compositions are provided comprising: (a) a retron-guide RNA cassette as provided herein, a vector of the present invention, a retron donor-DNA guide molecule, or a combination thereof; and (b) a pharmaceutically acceptable carrier.

In some embodiments provided herein, is a method for preventing or treating a genetic disease in a subject, the method comprising administering to the subject an effective amount of a pharmaceutical composition as provided herein to modify or alter a target gene associated with the genetic disease. It can also be used to modify a gene in a disease that will confer a benefit to the subject.

In some embodiments, methods a disease or condition are provided by modifying one or more target nucleic acids of interest at one or more target loci within a genome of a host cell, such as a mammalian cells. The term modifying as it relates to one or more target nucleic acids of interest at one or more target loci within a genome of a host cell means that the nucleotides can be replaced, inserted, or deleted. In some embodiments, a gene construct can be inserted to express a heterologous protein of interest.

In some embodiments, the methods comprise transforming the cell with a one or more vectors encoding a retron and a guide RNA (gRNA), which can be optionally, positioned to the 3′ or 5′ end of positioned of the retron when part of the same transcript, wherein the retron comprises: (i) an msr locus; (ii) a first inverted repeat sequence coding region; (iii) an msd locus; (iv) a donor DNA sequence located within the msd locus; and (v) a second inverted repeat sequence coding region; and (b) culturing the cell or transformed progeny of the host cell under conditions sufficient for expressing from the vector a retron donor DNA-guide molecule comprising a retron transcript and a guide RNA (gRNA) molecule to induce one or more sequence modifications at the one or more target loci within the genome.

As provided for herein, the retron comprising the msr/msd locus and the gRNA can be transformed or transduced into the cell on the same vector or a different vector. Even if in the same vector the retron and gRNA can be on the same contiguous transcript or the separate transcript. Thus, the cell can comprise a plurality of transcripts that contain the gRNA separate from the retron comprising the coding region with the msr/msd locus, which includes the mutated loci as provided for herein.

The compositions and methods provided for herein are suitable for any disease that has a genetic basis and/or is amenable to prevention or amelioration of disease-associated sequelae or symptoms by editing or correcting one or more genetic loci that are linked to the disease. Non-limiting examples of diseases include X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases. The compositions and methods can also be used to prevent or treat any combination of suitable genetic diseases.

In some embodiments, the subject is treated before any symptoms or sequelae of the genetic disease develop. In other embodiments, the subject has symptoms or sequelae of the genetic disease. In some instances, treatment results in a reduction or elimination of the symptoms or sequelae of the genetic disease.

In some embodiments, treatment includes administering compositions of the present invention directly to a subject. As a non-limiting example, pharmaceutical compositions of the present invention can be delivered directly to a subject (e.g., by local injection or systemic administration). In other embodiments, the compositions of the present invention are delivered to a host cell or population of host cells, and then the host cell or population of host cells is administered or transplanted to the subject. The host cell or population of host cells can be administered or transplanted with a pharmaceutically acceptable carrier. In some instances, editing of the host cell genome has not yet been completed prior to administration or transplantation to the subject. In other instances, editing of the host cell genome has been completed when administration or transplantation occurs. In certain instances, progeny of the host cell or population of host cells are transplanted into the subject. In some embodiments, correct editing of the host cell or population of host cells, or the progeny thereof, is verified before administering or transplanting edited cells or the progeny thereof into a subject. Procedures for transplantation, administration, and verification of correct genome editing are discussed herein and will be known to one of skill in the art.

Compositions provided herein also include cells and/or progeny thereof that have had their genomes edited by the methods and/or compositions of the present invention, may be administered as a single dose or as multiple doses, for example two doses administered at an interval of about one month, about two months, about three months, about six months or about 12 months. Other suitable dosage schedules can be determined by a medical practitioner.

Prevention or treatment can further comprise administering agents and/or performing procedures to prevent or treat concomitant or related conditions. As non-limiting examples, it may be necessary to administer drugs to suppress immune rejection of transplanted cells, or prevent or reduce inflammation or infection. A medical professional will readily be able to determine the appropriate concomitant therapies.

### ENUMERATED EMBODIMENTS

1. A method of modifying, or inducing one or more sequence modifications in one or more, target nucleic acids of interest at one or more target loci within a genome of a host cell, such as a mammalian cell, the method comprising:
(a) transforming the host cell with one or more vectors encoding a heterologous nucleic acid molecule and a guide RNA (gRNA), wherein the heterologous nucleic acid molecule comprises a first inverted repeat nucleic acid molecule sequence upstream of a coding region and a second inverted repeat nucleic acid molecule sequence downstream of the coding region, wherein the coding region comprises a nucleic acid molecule comprising:
(i) an msr locus and an msd locus comprising the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2; and
(ii) a donor DNA sequence within the msd locus,
wherein the msr locus and the msd locus form a RT binding region; and
(b) culturing the host cell or transformed progeny of the host cell under conditions sufficient for expressing from the one or more vectors the heterologous nucleic acid molecule comprising a retron transcript and a gRNA molecule to induce one or more sequence modifications in one or more target nucleic acids of interest at the one or more target loci within the genome.
2. The method of embodiment 1, wherein the nucleic acid molecule comprising the msr locus and the msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2.
3. The method of embodiment 2, wherein the nucleic acid molecule comprising the msr locus and the msd locus comprises the nucleic acid sequence of any one of SEQ ID NO: 1, or 2.
4. The method of embodiments 1-3, wherein the msr locus is a mutated msr locus and/or the msd locus is a mutated msd locus.
5 The method of embodiment 4, wherein the msd locus comprises a deletion of at least 1 nucleotide as compared to SEQ ID NO: 1, or 2.
6. The method of embodiment 5, wherein the msd locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to SEQ ID NO: 1, or 2.
7. The method of any one of embodiments 1-6, wherein the msd locus upstream (to the 5′ of) of the donor DNA sequence comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18.
8. The method of any one of embodiments 1-7, wherein the msd locus upstream (to the 5′ of) of the donor DNA sequence comprises a nucleic acid sequence of any one of SEQ ID NO: 4, 5, 6, 7, 8, 14, 15, 16, 17, or 18.
9. The method of any one of embodiments 1-8, wherein the msd locus downstream (to the 3′ of) of the donor DNA sequence comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23.
10. The method of any one of embodiments 1-9, wherein the msd locus downstream (to the 3′ of) of the donor DNA sequence comprises a nucleic acid sequence of any one of SEQ ID NO: 9, 10, 11, 12, 13, 19, 20, 21, 22, or 23.
11. The method of any one of embodiments 1-10, wherein the coding region comprises a nucleic acid molecule comprising:
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18, upstream of the donor DNA sequence; and
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23, downstream of the donor DNA sequence.
12. The method of any one of embodiments 1-11, wherein the coding region comprises a nucleic acid molecule comprising:
a nucleic acid sequence of any one of SEQ ID NO: 4, 5, 6, 7, 8, 14, 15, 16, 17, or 18, upstream of the donor DNA sequence; and
a nucleic acid sequence of any one of SEQ ID NO: 9, 10, 11, 12, 13, 19, 20, 21, 22, or 23, downstream of the donor DNA sequence.
13. The method of any one of embodiments 1-12, wherein the coding region comprises a nucleic acid molecule comprising:
a nucleic acid sequence of SEQ ID NO: 4 upstream of the donor DNA sequence; and a nucleic acid sequence of SEQ ID NO: 9 downstream of the donor DNA sequence;
a nucleic acid sequence of SEQ ID NO: 4 upstream of the donor DNA sequence; and a nucleic acid sequence of SEQ ID NO: 10 downstream of the donor DNA sequence;
a nucleic acid sequence of SEQ ID NO: 4 upstream of the donor DNA sequence; and a nucleic acid sequence of SEQ ID NO: 11 downstream of the donor DNA sequence;
a nucleic acid sequence of SEQ ID NO: 4 upstream of the donor DNA sequence; and a nucleic acid sequence of SEQ ID NO: 12 downstream of the donor DNA sequence;
a nucleic acid sequence of SEQ ID NO: 4 upstream of the donor DNA sequence; and a nucleic acid sequence of SEQ ID NO: 13 downstream of the donor DNA sequence;
a nucleic acid sequence of SEQ ID NO: 4 upstream of the donor DNA sequence; and a nucleic acid sequence of SEQ ID NO: 19 downstream of the donor DNA sequence;
a nucleic acid sequence of SEQ ID NO: 4 upstream of the donor DNA sequence; and a nucleic acid sequence of SEQ ID NO: 20 downstream of the donor DNA sequence;
a nucleic acid sequence of SEQ ID NO: 4 upstream of the donor DNA sequence; and a nucleic acid sequence of SEQ ID NO: 21 downstream of the donor DNA sequence;
a nucleic acid sequence of SEQ ID NO: 4 upstream of the donor DNA sequence; and a nucleic acid sequence of SEQ ID NO: 22 downstream of the donor DNA sequence;
a nucleic acid sequence of SEQ ID NO: 4 upstream of the donor DNA sequence; and a nucleic acid sequence of SEQ ID NO: 23 downstream of the donor DNA sequence;
a nucleic acid sequence of SEQ ID NO: 5 upstream of the donor DNA sequence; and a nucleic acid sequence of SEQ ID NO: 9 downstream of the donor DNA sequence;
a nucleic acid sequence of SEQ ID NO: 5 upstream of the donor DNA sequence; and a nucleic acid sequence of SEQ ID NO: 10 downstream of the donor DNA sequence;
a nucleic acid sequence of SEQ ID NO: 5 upstream of the donor DNA sequence; and a nucleic acid sequence of SEQ ID NO: 11 downstream of the donor DNA sequence;
a nucleic acid sequence of SEQ ID NO: 5 upstream of the donor DNA sequence; and a nucleic acid sequence of SEQ ID NO: 12 downstream of the donor DNA sequence;
a nucleic acid sequence of SEQ ID NO: 5 upstream of the donor DNA sequence; and a nucleic acid sequence of SEQ ID NO: 13 downstream of the donor DNA sequence;
a nucleic acid sequence of SEQ ID NO: 5 upstream of the donor DNA sequence; and a nucleic acid sequence of SEQ ID NO: 19 downstream of the donor DNA sequence;
a nucleic acid sequence of SEQ ID NO: 5 upstream of the donor DNA sequence; and a nucleic acid sequence of SEQ ID NO: 20 downstream of the donor DNA sequence;
a nucleic acid sequence of SEQ ID NO: 5 upstream of the donor DNA sequence; and a nucleic acid sequence of SEQ ID NO: 21 downstream of the donor DNA sequence;
a nucleic acid sequence of SEQ ID NO: 5 upstream of the donor DNA sequence; and a nucleic acid sequence of SEQ ID NO: 22 downstream of the donor DNA sequence;
a nucleic acid sequence of SEQ ID NO: 5 upstream of the donor DNA sequence; and a nucleic acid sequence of SEQ ID NO: 23 downstream of the donor DNA sequence;
a nucleic acid sequence of SEQ ID NO: 6 upstream of the donor DNA sequence; and a nucleic acid sequence of SEQ ID NO: 9 downstream of the donor DNA sequence;
a nucleic acid sequence of SEQ ID NO: 6 upstream of the donor DNA sequence; and a nucleic acid sequence of SEQ ID NO: 10 downstream of the donor DNA sequence;
a nucleic acid sequence of SEQ ID NO: 6 upstream of the donor DNA sequence; and a nucleic acid sequence of SEQ ID NO: 11 downstream of the donor DNA sequence;
a nucleic acid sequence of SEQ ID NO: 6 upstream of the donor DNA sequence; and a nucleic acid sequence of SEQ ID NO: 12 downstream of the donor DNA sequence;
a nucleic acid sequence of SEQ ID NO: 6 upstream of the donor DNA sequence; and a nucleic acid sequence of SEQ ID NO: 13 downstream of the donor DNA sequence;
a nucleic acid sequence of SEQ ID NO: 6 upstream of the donor DNA sequence; and a nucleic acid sequence of SEQ ID NO: 19 downstream of the donor DNA sequence;
a nucleic acid sequence of SEQ ID NO: 6 upstream of the donor DNA sequence; and a nucleic acid sequence of SEQ ID NO: 20 downstream of the donor DNA sequence;
a nucleic acid sequence of SEQ ID NO: 6 upstream of the donor DNA sequence; and a nucleic acid sequence of SEQ ID NO: 21 downstream of the donor DNA sequence;
a nucleic acid sequence of SEQ ID NO: 6 upstream of the donor DNA sequence; and a nucleic acid sequence of SEQ ID NO: 22 downstream of the donor DNA sequence;
a nucleic acid sequence of SEQ ID NO: 6 upstream of the donor DNA sequence; and a nucleic acid sequence of SEQ ID NO: 23 downstream of the donor DNA sequence;
a nucleic acid sequence of SEQ ID NO: 7 upstream of the donor DNA sequence; and a nucleic acid sequence of SEQ ID NO: 9 downstream of the donor DNA sequence;
a nucleic acid sequence of SEQ ID NO: 7 upstream of the donor DNA sequence; and a nucleic acid sequence of SEQ ID NO: 10 downstream of the donor DNA sequence;
a nucleic acid sequence of SEQ ID NO: 7 upstream of the donor DNA sequence; and a nucleic acid sequence of SEQ ID NO: 11 downstream of the donor DNA sequence;
a nucleic acid sequence of SEQ ID NO: 7 upstream of the donor DNA sequence; and a nucleic acid sequence of SEQ ID NO: 12 downstream of the donor DNA sequence;
a nucleic acid sequence of SEQ ID NO: 7 upstream of the donor DNA sequence; and a nucleic acid sequence of SEQ ID NO: 13 downstream of the donor DNA sequence;
a nucleic acid sequence of SEQ ID NO: 7 upstream of the donor DNA sequence; and a nucleic acid sequence of SEQ ID NO: 19 downstream of the donor DNA sequence;
a nucleic acid sequence of SEQ ID NO: 7 upstream of the donor DNA sequence; and a nucleic acid sequence of SEQ ID NO: 20 downstream of the donor DNA sequence;
a nucleic acid sequence of SEQ ID NO: 7 upstream of the donor DNA sequence; and a nucleic acid sequence of SEQ ID NO: 21 downstream of the donor DNA sequence;
a nucleic acid sequence of SEQ ID NO: 7 upstream of the donor DNA sequence; and a nucleic acid sequence of SEQ ID NO: 22 downstream of the donor DNA sequence;
a nucleic acid sequence of SEQ ID NO: 7 upstream of the donor DNA sequence; and a nucleic acid sequence of SEQ ID NO: 23 downstream of the donor DNA sequence;
a nucleic acid sequence of SEQ ID NO: 8 upstream of the donor DNA sequence; and a nucleic acid sequence of SEQ ID NO: 9 downstream of the donor DNA sequence;
a nucleic acid sequence of SEQ ID NO: 8 upstream of the donor DNA sequence; and a nucleic acid sequence of SEQ ID NO: 10 downstream of the donor DNA sequence;
a nucleic acid sequence of SEQ ID NO: 8 upstream of the donor DNA sequence; and a nucleic acid sequence of SEQ ID NO: 11 downstream of the donor DNA sequence;
a nucleic acid sequence of SEQ ID NO: 8 upstream of the donor DNA sequence; and a nucleic acid sequence of SEQ ID NO: 12 downstream of the donor DNA sequence;
a nucleic acid sequence of SEQ ID NO: 8 upstream of the donor DNA sequence; and a nucleic acid sequence of SEQ ID NO: 13 downstream of the donor DNA sequence;
a nucleic acid sequence of SEQ ID NO: 8 upstream of the donor DNA sequence; and a nucleic acid sequence of SEQ ID NO: 19 downstream of the donor DNA sequence;
a nucleic acid sequence of SEQ ID NO: 8 upstream of the donor DNA sequence; and a nucleic acid sequence of SEQ ID NO: 20 downstream of the donor DNA sequence;
a nucleic acid sequence of SEQ ID NO: 8 upstream of the donor DNA sequence; and a nucleic acid sequence of SEQ ID NO: 21 downstream of the donor DNA sequence;
a nucleic acid sequence of SEQ ID NO: 8 upstream of the donor DNA sequence; and a nucleic acid sequence of SEQ ID NO: 22 downstream of the donor DNA sequence;
a nucleic acid sequence of SEQ ID NO: 8 upstream of the donor DNA sequence; and a nucleic acid sequence of SEQ ID NO: 23 downstream of the donor DNA sequence;
a nucleic acid sequence of SEQ ID NO: 14 upstream of the donor DNA sequence; and a nucleic acid sequence of SEQ ID NO: 9 downstream of the donor DNA sequence;
a nucleic acid sequence of SEQ ID NO: 14 upstream of the donor DNA sequence; and a nucleic acid sequence of SEQ ID NO: 10 downstream of the donor DNA sequence;
a nucleic acid sequence of SEQ ID NO: 14 upstream of the donor DNA sequence; and a nucleic acid sequence of SEQ ID NO: 11 downstream of the donor DNA sequence;
a nucleic acid sequence of SEQ ID NO: 14 upstream of the donor DNA sequence; and a nucleic acid sequence of SEQ ID NO: 12 downstream of the donor DNA sequence;
a nucleic acid sequence of SEQ ID NO: 14 upstream of the donor DNA sequence; and a nucleic acid sequence of SEQ ID NO: 13 downstream of the donor DNA sequence;
a nucleic acid sequence of SEQ ID NO: 14 upstream of the donor DNA sequence; and a nucleic acid sequence of SEQ ID NO: 19 downstream of the donor DNA sequence;
a nucleic acid sequence of SEQ ID NO: 14 upstream of the donor DNA sequence; and a nucleic acid sequence of SEQ ID NO: 20 downstream of the donor DNA sequence;
a nucleic acid sequence of SEQ ID NO: 14 upstream of the donor DNA sequence; and a nucleic acid sequence of SEQ ID NO: 21 downstream of the donor DNA sequence;
a nucleic acid sequence of SEQ ID NO: 14 upstream of the donor DNA sequence; and a nucleic acid sequence of SEQ ID NO: 22 downstream of the donor DNA sequence;
a nucleic acid sequence of SEQ ID NO: 14 upstream of the donor DNA sequence; and a nucleic acid sequence of SEQ ID NO: 23 downstream of the donor DNA sequence;
a nucleic acid sequence of SEQ ID NO: 15 upstream of the donor DNA sequence; and a nucleic acid sequence of SEQ ID NO: 9 downstream of the donor DNA sequence;
a nucleic acid sequence of SEQ ID NO: 15 upstream of the donor DNA sequence; and a nucleic acid sequence of SEQ ID NO: 10 downstream of the donor DNA sequence;
a nucleic acid sequence of SEQ ID NO: 15 upstream of the donor DNA sequence; and a nucleic acid sequence of SEQ ID NO: 11 downstream of the donor DNA sequence;
a nucleic acid sequence of SEQ ID NO: 15 upstream of the donor DNA sequence; and a nucleic acid sequence of SEQ ID NO: 12 downstream of the donor DNA sequence;
a nucleic acid sequence of SEQ ID NO: 15 upstream of the donor DNA sequence; and a nucleic acid sequence of SEQ ID NO: 13 downstream of the donor DNA sequence;
a nucleic acid sequence of SEQ ID NO: 15 upstream of the donor DNA sequence; and a nucleic acid sequence of SEQ ID NO: 19 downstream of the donor DNA sequence;
a nucleic acid sequence of SEQ ID NO: 15 upstream of the donor DNA sequence; and a nucleic acid sequence of SEQ ID NO: 20 downstream of the donor DNA sequence;
a nucleic acid sequence of SEQ ID NO: 15 upstream of the donor DNA sequence; and a nucleic acid sequence of SEQ ID NO: 21 downstream of the donor DNA sequence;
a nucleic acid sequence of SEQ ID NO: 15 upstream of the donor DNA sequence; and a nucleic acid sequence of SEQ ID NO: 22 downstream of the donor DNA sequence;
a nucleic acid sequence of SEQ ID NO: 15 upstream of the donor DNA sequence; and a nucleic acid sequence of SEQ ID NO: 23 downstream of the donor DNA sequence;
a nucleic acid sequence of SEQ ID NO: 16 upstream of the donor DNA sequence; and a nucleic acid sequence of SEQ ID NO: 9 downstream of the donor DNA sequence;
a nucleic acid sequence of SEQ ID NO: 16 upstream of the donor DNA sequence; and a nucleic acid sequence of SEQ ID NO: 10 downstream of the donor DNA sequence;
a nucleic acid sequence of SEQ ID NO: 16 upstream of the donor DNA sequence; and a nucleic acid sequence of SEQ ID NO: 11 downstream of the donor DNA sequence;
a nucleic acid sequence of SEQ ID NO: 16 upstream of the donor DNA sequence; and a nucleic acid sequence of SEQ ID NO: 12 downstream of the donor DNA sequence;
a nucleic acid sequence of SEQ ID NO: 16 upstream of the donor DNA sequence; and a nucleic acid sequence of SEQ ID NO: 13 downstream of the donor DNA sequence;
a nucleic acid sequence of SEQ ID NO: 16 upstream of the donor DNA sequence; and a nucleic acid sequence of SEQ ID NO: 19 downstream of the donor DNA sequence;
a nucleic acid sequence of SEQ ID NO: 16 upstream of the donor DNA sequence; and a nucleic acid sequence of SEQ ID NO: 20 downstream of the donor DNA sequence;
a nucleic acid sequence of SEQ ID NO: 16 upstream of the donor DNA sequence; and a nucleic acid sequence of SEQ ID NO: 21 downstream of the donor DNA sequence;
a nucleic acid sequence of SEQ ID NO: 16 upstream of the donor DNA sequence; and a nucleic acid sequence of SEQ ID NO: 22 downstream of the donor DNA sequence;
a nucleic acid sequence of SEQ ID NO: 16 upstream of the donor DNA sequence; and a nucleic acid sequence of SEQ ID NO: 23 downstream of the donor DNA sequence;
a nucleic acid sequence of SEQ ID NO: 17 upstream of the donor DNA sequence; and a nucleic acid sequence of SEQ ID NO: 9 downstream of the donor DNA sequence;
a nucleic acid sequence of SEQ ID NO: 17 upstream of the donor DNA sequence; and a nucleic acid sequence of SEQ ID NO: 10 downstream of the donor DNA sequence;
a nucleic acid sequence of SEQ ID NO: 17 upstream of the donor DNA sequence; and a nucleic acid sequence of SEQ ID NO: 11 downstream of the donor DNA sequence;
a nucleic acid sequence of SEQ ID NO: 17 upstream of the donor DNA sequence; and a nucleic acid sequence of SEQ ID NO: 12 downstream of the donor DNA sequence;
a nucleic acid sequence of SEQ ID NO: 17 upstream of the donor DNA sequence; and a nucleic acid sequence of SEQ ID NO: 13 downstream of the donor DNA sequence;
a nucleic acid sequence of SEQ ID NO: 17 upstream of the donor DNA sequence; and a nucleic acid sequence of SEQ ID NO: 19 downstream of the donor DNA sequence;
a nucleic acid sequence of SEQ ID NO: 17 upstream of the donor DNA sequence; and a nucleic acid sequence of SEQ ID NO: 20 downstream of the donor DNA sequence;
a nucleic acid sequence of SEQ ID NO: 17 upstream of the donor DNA sequence; and a nucleic acid sequence of SEQ ID NO: 21 downstream of the donor DNA sequence;
a nucleic acid sequence of SEQ ID NO: 17 upstream of the donor DNA sequence; and a nucleic acid sequence of SEQ ID NO: 22 downstream of the donor DNA sequence;
a nucleic acid sequence of SEQ ID NO: 17 upstream of the donor DNA sequence; and a nucleic acid sequence of SEQ ID NO: 23 downstream of the donor DNA sequence;
a nucleic acid sequence of SEQ ID NO: 18 upstream of the donor DNA sequence; and a nucleic acid sequence of SEQ ID NO: 9 downstream of the donor DNA sequence;
a nucleic acid sequence of SEQ ID NO: 18 upstream of the donor DNA sequence; and a nucleic acid sequence of SEQ ID NO: 10 downstream of the donor DNA sequence;
a nucleic acid sequence of SEQ ID NO: 18 upstream of the donor DNA sequence; and a nucleic acid sequence of SEQ ID NO: 11 downstream of the donor DNA sequence;
a nucleic acid sequence of SEQ ID NO: 18 upstream of the donor DNA sequence; and a nucleic acid sequence of SEQ ID NO: 12 downstream of the donor DNA sequence;
a nucleic acid sequence of SEQ ID NO: 18 upstream of the donor DNA sequence; and a nucleic acid sequence of SEQ ID NO: 13 downstream of the donor DNA sequence;
a nucleic acid sequence of SEQ ID NO: 18 upstream of the donor DNA sequence; and a nucleic acid sequence of SEQ ID NO: 19 downstream of the donor DNA sequence;
a nucleic acid sequence of SEQ ID NO: 18 upstream of the donor DNA sequence; and a nucleic acid sequence of SEQ ID NO: 20 downstream of the donor DNA sequence;
a nucleic acid sequence of SEQ ID NO: 18 upstream of the donor DNA sequence; and a nucleic acid sequence of SEQ ID NO: 21 downstream of the donor DNA sequence;
a nucleic acid sequence of SEQ ID NO: 18 upstream of the donor DNA sequence; and a nucleic acid sequence of SEQ ID NO: 22 downstream of the donor DNA sequence; or
a nucleic acid sequence of SEQ ID NO: 18 upstream of the donor DNA sequence; and a nucleic acid sequence of SEQ ID NO: 23 downstream of the donor DNA sequence.
14. The method of embodiment 1, wherein the coding region comprises a nucleic acid molecule having a formula of 5′-M1-X1-M2-3′, wherein M1 is a fragment of the msr/msd locus, X1 is the donor DNA sequence coding region; and M2 is a fragment of the msd/msr locus.
15. The method of embodiment 14, wherein M1 comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33.
16. The method of embodiment 15, wherein M1 comprises a nucleic acid sequence of any one of SEQ ID NO: 24, 25, 26, 27, 28, 29, 30, 31, 32, or 33.
17. The method of embodiment 14, wherein M2 comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.
18. The method of embodiment 17, wherein M2 comprises a nucleic acid sequence of any one of SEQ ID NO: 34, 35, 36, 37, 38, 39, 40, 41, 42, or 43.
19. The method of any one of embodiments 14-18, wherein the nucleic acid molecule comprises an M1 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and an M2 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.
20. The method of embodiment 19, wherein the nucleic acid molecule comprises
an M1 having a nucleic acid sequence of SEQ ID NO: 24; and an M2 having a nucleic acid sequence of SEQ ID NO: 34;
an M1 having a nucleic acid sequence of SEQ ID NO: 24; and an M2 having a nucleic acid sequence of SEQ ID NO: 35;
an M1 having a nucleic acid sequence of SEQ ID NO: 24; and an M2 having a nucleic acid sequence of SEQ ID NO: 36;
an M1 having a nucleic acid sequence of SEQ ID NO: 24; and an M2 having a nucleic acid sequence of SEQ ID NO: 37;
an M1 having a nucleic acid sequence of SEQ ID NO: 24; and an M2 having a nucleic acid sequence of SEQ ID NO: 38;
an M1 having a nucleic acid sequence of SEQ ID NO: 24; and an M2 having a nucleic acid sequence of SEQ ID NO: 39;
an M1 having a nucleic acid sequence of SEQ ID NO: 24; and an M2 having a nucleic acid sequence of SEQ ID NO: 40;
an M1 having a nucleic acid sequence of SEQ ID NO: 24; and an M2 having a nucleic acid sequence of SEQ ID NO: 41;
an M1 having a nucleic acid sequence of SEQ ID NO: 24; and an M2 having a nucleic acid sequence of SEQ ID NO: 42;
an M1 having a nucleic acid sequence of SEQ ID NO: 24; and an M2 having a nucleic acid sequence of SEQ ID NO: 43;
an M1 having a nucleic acid sequence of SEQ ID NO: 25; and an M2 having a nucleic acid sequence of SEQ ID NO: 34;
an M1 having a nucleic acid sequence of SEQ ID NO: 25; and an M2 having a nucleic acid sequence of SEQ ID NO: 35;
an M1 having a nucleic acid sequence of SEQ ID NO: 25; and an M2 having a nucleic acid sequence of SEQ ID NO: 36;
an M1 having a nucleic acid sequence of SEQ ID NO: 25; and an M2 having a nucleic acid sequence of SEQ ID NO: 37;
an M1 having a nucleic acid sequence of SEQ ID NO: 25; and an M2 having a nucleic acid sequence of SEQ ID NO: 38;
an M1 having a nucleic acid sequence of SEQ ID NO: 25; and an M2 having a nucleic acid sequence of SEQ ID NO: 39;
an M1 having a nucleic acid sequence of SEQ ID NO: 25; and an M2 having a nucleic acid sequence of SEQ ID NO: 40;
an M1 having a nucleic acid sequence of SEQ ID NO: 25; and an M2 having a nucleic acid sequence of SEQ ID NO: 41;
an M1 having a nucleic acid sequence of SEQ ID NO: 25; and an M2 having a nucleic acid sequence of SEQ ID NO: 42;
an M1 having a nucleic acid sequence of SEQ ID NO: 25; and an M2 having a nucleic acid sequence of SEQ ID NO: 43;
an M1 having a nucleic acid sequence of SEQ ID NO: 26; and an M2 having a nucleic acid sequence of SEQ ID NO: 34;
an M1 having a nucleic acid sequence of SEQ ID NO: 26; and an M2 having a nucleic acid sequence of SEQ ID NO: 35;
an M1 having a nucleic acid sequence of SEQ ID NO: 26; and an M2 having a nucleic acid sequence of SEQ ID NO: 36;
an M1 having a nucleic acid sequence of SEQ ID NO: 26; and an M2 having a nucleic acid sequence of SEQ ID NO: 37;
an M1 having a nucleic acid sequence of SEQ ID NO: 26; and an M2 having a nucleic acid sequence of SEQ ID NO: 38;
an M1 having a nucleic acid sequence of SEQ ID NO: 26; and an M2 having a nucleic acid sequence of SEQ ID NO: 39;
an M1 having a nucleic acid sequence of SEQ ID NO: 26; and an M2 having a nucleic acid sequence of SEQ ID NO: 40;
an M1 having a nucleic acid sequence of SEQ ID NO: 26; and an M2 having a nucleic acid sequence of SEQ ID NO: 41;
an M1 having a nucleic acid sequence of SEQ ID NO: 26; and an M2 having a nucleic acid sequence of SEQ ID NO: 42;
an M1 having a nucleic acid sequence of SEQ ID NO: 26; and an M2 having a nucleic acid sequence of SEQ ID NO: 43;
an M1 having a nucleic acid sequence of SEQ ID NO: 27; and an M2 having a nucleic acid sequence of SEQ ID NO: 34;
an M1 having a nucleic acid sequence of SEQ ID NO: 27; and an M2 having a nucleic acid sequence of SEQ ID NO: 35;
an M1 having a nucleic acid sequence of SEQ ID NO: 27; and an M2 having a nucleic acid sequence of SEQ ID NO: 36;
an M1 having a nucleic acid sequence of SEQ ID NO: 27; and an M2 having a nucleic acid sequence of SEQ ID NO: 37;
an M1 having a nucleic acid sequence of SEQ ID NO: 27; and an M2 having a nucleic acid sequence of SEQ ID NO: 38;
an M1 having a nucleic acid sequence of SEQ ID NO: 27; and an M2 having a nucleic acid sequence of SEQ ID NO: 39;
an M1 having a nucleic acid sequence of SEQ ID NO: 27; and an M2 having a nucleic acid sequence of SEQ ID NO: 40;
an M1 having a nucleic acid sequence of SEQ ID NO: 27; and an M2 having a nucleic acid sequence of SEQ ID NO: 41;
an M1 having a nucleic acid sequence of SEQ ID NO: 27; and an M2 having a nucleic acid sequence of SEQ ID NO: 42;
an M1 having a nucleic acid sequence of SEQ ID NO: 27; and an M2 having a nucleic acid sequence of SEQ ID NO: 43;
an M1 having a nucleic acid sequence of SEQ ID NO: 28; and an M2 having a nucleic acid sequence of SEQ ID NO: 34;
an M1 having a nucleic acid sequence of SEQ ID NO: 28; and an M2 having a nucleic acid sequence of SEQ ID NO: 35;
an M1 having a nucleic acid sequence of SEQ ID NO: 28; and an M2 having a nucleic acid sequence of SEQ ID NO: 36;
an M1 having a nucleic acid sequence of SEQ ID NO: 28; and an M2 having a nucleic acid sequence of SEQ ID NO: 37;
an M1 having a nucleic acid sequence of SEQ ID NO: 28; and an M2 having a nucleic acid sequence of SEQ ID NO: 38;
an M1 having a nucleic acid sequence of SEQ ID NO: 28; and an M2 having a nucleic acid sequence of SEQ ID NO: 39;
an M1 having a nucleic acid sequence of SEQ ID NO: 28; and an M2 having a nucleic acid sequence of SEQ ID NO: 40;
an M1 having a nucleic acid sequence of SEQ ID NO: 28; and an M2 having a nucleic acid sequence of SEQ ID NO: 41;
an M1 having a nucleic acid sequence of SEQ ID NO: 28; and an M2 having a nucleic acid sequence of SEQ ID NO: 42;
an M1 having a nucleic acid sequence of SEQ ID NO: 28; and an M2 having a nucleic acid sequence of SEQ ID NO: 43;
an M1 having a nucleic acid sequence of SEQ ID NO: 29; and an M2 having a nucleic acid sequence of SEQ ID NO: 34;
an M1 having a nucleic acid sequence of SEQ ID NO: 29; and an M2 having a nucleic acid sequence of SEQ ID NO: 35;
an M1 having a nucleic acid sequence of SEQ ID NO: 29; and an M2 having a nucleic acid sequence of SEQ ID NO: 36;
an M1 having a nucleic acid sequence of SEQ ID NO: 29; and an M2 having a nucleic acid sequence of SEQ ID NO: 37;
an M1 having a nucleic acid sequence of SEQ ID NO: 29; and an M2 having a nucleic acid sequence of SEQ ID NO: 38;
an M1 having a nucleic acid sequence of SEQ ID NO: 29; and an M2 having a nucleic acid sequence of SEQ ID NO: 39;
an M1 having a nucleic acid sequence of SEQ ID NO: 29; and an M2 having a nucleic acid sequence of SEQ ID NO: 40;
an M1 having a nucleic acid sequence of SEQ ID NO: 29; and an M2 having a nucleic acid sequence of SEQ ID NO: 41;
an M1 having a nucleic acid sequence of SEQ ID NO: 29; and an M2 having a nucleic acid sequence of SEQ ID NO: 42;
an M1 having a nucleic acid sequence of SEQ ID NO: 29; and an M2 having a nucleic acid sequence of SEQ ID NO: 43;
an M1 having a nucleic acid sequence of SEQ ID NO: 30; and an M2 having a nucleic acid sequence of SEQ ID NO: 34;
an M1 having a nucleic acid sequence of SEQ ID NO: 30; and an M2 having a nucleic acid sequence of SEQ ID NO: 35;
an M1 having a nucleic acid sequence of SEQ ID NO: 30; and an M2 having a nucleic acid sequence of SEQ ID NO: 36;
an M1 having a nucleic acid sequence of SEQ ID NO: 30; and an M2 having a nucleic acid sequence of SEQ ID NO: 37;
an M1 having a nucleic acid sequence of SEQ ID NO: 30; and an M2 having a nucleic acid sequence of SEQ ID NO: 38;
an M1 having a nucleic acid sequence of SEQ ID NO: 30; and an M2 having a nucleic acid sequence of SEQ ID NO: 39;
an M1 having a nucleic acid sequence of SEQ ID NO: 30; and an M2 having a nucleic acid sequence of SEQ ID NO: 40;
an M1 having a nucleic acid sequence of SEQ ID NO: 30; and an M2 having a nucleic acid sequence of SEQ ID NO: 41;
an M1 having a nucleic acid sequence of SEQ ID NO: 30; and an M2 having a nucleic acid sequence of SEQ ID NO: 42;
an M1 having a nucleic acid sequence of SEQ ID NO: 30; and an M2 having a nucleic acid sequence of SEQ ID NO: 43;
an M1 having a nucleic acid sequence of SEQ ID NO: 31; and an M2 having a nucleic acid sequence of SEQ ID NO: 34;
an M1 having a nucleic acid sequence of SEQ ID NO: 31; and an M2 having a nucleic acid sequence of SEQ ID NO: 35;
an M1 having a nucleic acid sequence of SEQ ID NO: 31; and an M2 having a nucleic acid sequence of SEQ ID NO: 36;
an M1 having a nucleic acid sequence of SEQ ID NO: 31; and an M2 having a nucleic acid sequence of SEQ ID NO: 37;
an M1 having a nucleic acid sequence of SEQ ID NO: 31; and an M2 having a nucleic acid sequence of SEQ ID NO: 38;
an M1 having a nucleic acid sequence of SEQ ID NO: 31; and an M2 having a nucleic acid sequence of SEQ ID NO: 39;
an M1 having a nucleic acid sequence of SEQ ID NO: 31; and an M2 having a nucleic acid sequence of SEQ ID NO: 40;
an M1 having a nucleic acid sequence of SEQ ID NO: 31; and an M2 having a nucleic acid sequence of SEQ ID NO: 41;
an M1 having a nucleic acid sequence of SEQ ID NO: 31; and an M2 having a nucleic acid sequence of SEQ ID NO: 42;
an M1 having a nucleic acid sequence of SEQ ID NO: 31; and an M2 having a nucleic acid sequence of SEQ ID NO: 43;
an M1 having a nucleic acid sequence of SEQ ID NO: 32; and an M2 having a nucleic acid sequence of SEQ ID NO: 34;
an M1 having a nucleic acid sequence of SEQ ID NO: 32; and an M2 having a nucleic acid sequence of SEQ ID NO: 35;
an M1 having a nucleic acid sequence of SEQ ID NO: 32; and an M2 having a nucleic acid sequence of SEQ ID NO: 36;
an M1 having a nucleic acid sequence of SEQ ID NO: 32; and an M2 having a nucleic acid sequence of SEQ ID NO: 37;
an M1 having a nucleic acid sequence of SEQ ID NO: 32; and an M2 having a nucleic acid sequence of SEQ ID NO: 38;
an M1 having a nucleic acid sequence of SEQ ID NO: 32; and an M2 having a nucleic acid sequence of SEQ ID NO: 39;
an M1 having a nucleic acid sequence of SEQ ID NO: 32; and an M2 having a nucleic acid sequence of SEQ ID NO: 40;
an M1 having a nucleic acid sequence of SEQ ID NO: 32; and an M2 having a nucleic acid sequence of SEQ ID NO: 41;
an M1 having a nucleic acid sequence of SEQ ID NO: 32; and an M2 having a nucleic acid sequence of SEQ ID NO: 42;
an M1 having a nucleic acid sequence of SEQ ID NO: 32; and an M2 having a nucleic acid sequence of SEQ ID NO: 43;
an M1 having a nucleic acid sequence of SEQ ID NO: or 33; and an M2 having a nucleic acid sequence of SEQ ID NO: 34;
an M1 having a nucleic acid sequence of SEQ ID NO: or 33; and an M2 having a nucleic acid sequence of SEQ ID NO: 35;
an M1 having a nucleic acid sequence of SEQ ID NO: or 33; and an M2 having a nucleic acid sequence of SEQ ID NO: 36;
an M1 having a nucleic acid sequence of SEQ ID NO: or 33; and an M2 having a nucleic acid sequence of SEQ ID NO: 37;
an M1 having a nucleic acid sequence of SEQ ID NO: or 33; and an M2 having a nucleic acid sequence of SEQ ID NO: 38;
an M1 having a nucleic acid sequence of SEQ ID NO: or 33; and an M2 having a nucleic acid sequence of SEQ ID NO: 39;
an M1 having a nucleic acid sequence of SEQ ID NO: or 33; and an M2 having a nucleic acid sequence of SEQ ID NO: 40;
an M1 having a nucleic acid sequence of SEQ ID NO: or 33; and an M2 having a nucleic acid sequence of SEQ ID NO: 41;
an M1 having a nucleic acid sequence of SEQ ID NO: or 33; and an M2 having a nucleic acid sequence of SEQ ID NO: 42; or an M1 having a nucleic acid sequence of SEQ ID NO: or 33; and an M2 having a nucleic acid sequence of SEQ ID NO: 43.
21. The method of embodiment 1, wherein the donor DNA sequence is a target strand donor DNA sequence.
22. The method of embodiment 1, wherein the donor DNA sequence is a non-target strand donor DNA sequence.
23. The method of any one of embodiments 1-22, wherein the host cell expresses a nuclease and a RT protein.
24. The method embodiment 23, wherein the nuclease and RT are not expressed as a fusion molecule.
25. The method embodiment 23, wherein the nuclease and RT are each expressed as separate molecules.
26. The method embodiment 23, wherein the nuclease and RT are expressed as a fusion protein.
27 The method of embodiments 23 or 26, wherein the nuclease is linked to the RT with a linker.
28. The method of embodiment 27, wherein the linker is a peptide linker.
29. The method of any one of embodiments 26-28, wherein the C-terminus of the nuclease is linked to the N-terminus of the RT.
30. The method of any one of embodiments 26-28, wherein the N-terminus of the nuclease is linked to the C-terminus of the RT.
31. The method of any one of embodiments 1-30, wherein the host cell is transfected with a vector expressing the nuclease and/or the RT.
32. The method of any one of embodiments 1-31, wherein the host cell stably expresses the nuclease and/or the RT.
33. The method of any one of embodiments 1-32, wherein the host cell expresses a RT and a single-stranded annealing protein (SSAP).
34. The method of embodiment 33, wherein the RT and the SSAP are not expressed as a fusion molecule.
35. The method of embodiment 33, wherein the RT and the SSAP are each expressed as separate molecules.
36. The method of embodiment 33, wherein the RT and the SSAP are expressed as a fusion molecule.
37. The method of embodiment 36, wherein the RT and the SSAP are linked to one another with a linker.
38. The method of embodiment 37, wherein the linker is a peptide linker.
39. The method of any one of embodiments 36-38, wherein the C-terminus of the SSAP is linked to the N-terminus of the RT.
40. The method of any one of embodiments 36-38, wherein the N-terminus of the SSAP is linked to the C-terminus of the RT.
41. The method of any one of embodiments 22-40, wherein the nuclease is a Cas nuclease.
42. The method of embodiment 41, wherein the Cas nuclease is a catalytically active nuclease, a nickase Cas nuclease, or a catalytically inactive Cas nuclease.
43. The method of any one of embodiments 41-42, wherein the Cas nuclease is a Cas9 nuclease, nickase Cas9 nuclease, or a catalytically inactive Cas9 nuclease.
44. The method of embodiment 43, wherein the nickase Cas9 nuclease is Cas9D10A or Cas9H840A nuclease or a Cas having the both D10A and H840A mutations.
45. The method of any one of embodiments 33-44, wherein the RT, the SSAP and the nuclease are not expressed as a fusion protein.
46. The method of any one of embodiments 33-44, wherein the RT, the SSAP and the nuclease are each expressed as separate molecules.
47. The method of any one of embodiments 33-44, wherein the RT, the SSAP and the nuclease are expressed as a fusion protein.
48. The method of embodiment 47, wherein the RT, the SSAP and the nuclease are linked to one another with a linker.
49. The method of embodiment 48, wherein the linker is a peptide linker.
50. The method of any one of embodiments 47-49, wherein the C-terminus of the RT is linked to the N-terminus of the nuclease, and the C-terminus of the nuclease is linked to the N-terminus of the SSAP.
51. The method of any one of embodiments 47-49, wherein the N-terminus of the RT is linked to the C-terminus of the nuclease, and the N-terminus of the nuclease is linked to the C-terminus of the SSAP.
52. The method of any one of embodiments 22-51, wherein the RT is a fusion protein comprising a polypeptide having the formula of: of (N₁)_{q}-C₁-L₁-(N₂)_{qq}-R₁-(N₃)_{qqq}, wherein C1 is a nuclease, L1 is a peptide linker, R1 is a RT protein, and N1 and N2 are each independently, a NLS sequence, and wherein q, qq, or qqq, are each independently, 0, 1, 2, or 3.
53. The method of embodiment 52, wherein the q of 2 comprises a first NLS sequence linked to a second NLS sequence with a linker.
54. The method of any one of embodiments 52-53, wherein the first NLS sequence is identical to the second NLS sequence.
55. The method of any one of embodiments 52-53, wherein the first NLS sequence is different than the second NLS sequence.
56. The method of embodiment 52, wherein the qq of 1 comprises the NLS sequence linked at the C-terminus end of the NLS sequence to the N-terminus to R1.
57 The method of any one of embodiments 52-56, wherein the polypeptide has the formula of:

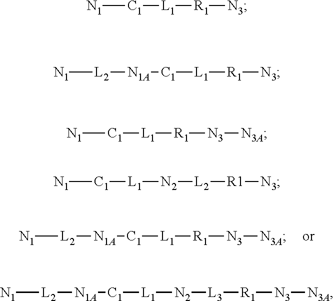

wherein:
each N1, N1A, N2, N3, or N3A is, independently, a NLS sequence;
   each L1, L2, or L3 is independently, a linker amino acid sequence; and
wherein each NLS sequence is identical or different.
58. The method of any one of embodiments 52-57, wherein N₁, N_{1A}, N₂, N₃, or N_{3A}, each independently comprise a SV40 NLS sequence, a cMyc NLS sequence, or a Nuceloplasmin NLS sequence.
59. The method of any one of embodiments 52-58, wherein the N₁ or the N_{1A} comprises the cMyc NLS sequence or the SV40 NLS sequence.
60. The method of any one of embodiments 52-58, wherein the N₂ comprises the SV40 NLS sequence or the Nuceloplasmin NLS sequence.
61. The method of any one of embodiments 52-58, wherein the N₃ or the N_{3A} comprises the Nuceloplasmin NLS sequence.
62. The method of any one of embodiments 52-61, wherein the linker amino acid sequence each independently comprise an XTEN linker, a G/S linker, a G/A linker, or any combination thereof.
63. The method of any one of embodiments 52-62, wherein the Cas comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 69.
64. The method of any one of embodiments 52-63, wherein the Cas comprises an amino acid sequence of SEQ ID NO: 69.
65. The method of any one of embodiments 52-64, wherein R1 comprises a RT sequence Ec86, Ec48, Ec73, Ec107, or Sa163.
66. The method of any one of embodiments 52-65, wherein the polypeptide comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74.
67. The method of any one of embodiments 52-66, wherein the polypeptide comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74, provided that the polypeptide comprises 1, 2, 3, or 4 NLS amino acid sequences.
68. The method of any one of embodiments 52-67, wherein the polypeptide comprises an amino acid sequence of any one of SEQ ID NO: 70, 71, 72, 73, or 74.
69. The method of embodiment 68, wherein the polypeptide comprises a domain that has an RT activity and a nuclease activity.
70. The method of any one of embodiments 1-69, wherein the retron transcript and the gRNA are not linked together.
71. The method of any one of embodiments 1-69, wherein the retron transcript and the gRNA are linked together.
72. The method of embodiment 71, wherein the retron transcript and the gRNA are linked together as part of the same transcript.
73. The method of embodiment 71, wherein the retron transcript and the gRNA are linked together as not part of the same contiguous transcript.
74. The method of embodiment 71, wherein the retron and gRNA are covalently linked together.
75. The method of embodiments 71-74, wherein the retron transcript and gRNA are linked together with a linker.
76. The method of embodiment 75, wherein the linker is a peptide linker.
77. The method of embodiment 76, wherein the peptide linker comprises an amino acid sequence of (GAA)n, wherein n is 1-20, 1-15, 1-10, 1-5, or 1-3.
78. The method of any one of embodiments 1-77, wherein the retron transcript and the gRNA comprises a structured motif at a 5′ or a 3′ end of the retron transcript and the gRNA.
79. The method of embodiment 78, wherein the structured motif is at the 5′ end of the retron transcript and the gRNA.
80. The method of any one of embodiments 78-79, wherein the structured motif is at the 3′ end of the retron transcript and the gRNA.
81. The method of any one of embodiments 78-80, wherein the structured motif stabilizes the transcript.
82. The method of any one of embodiments 78-81, wherein the structured motif is, independently, evoPreQ, tevopreQ mpKnot, or exoribonuclease-resistant RNA motif.
83. The method of any one of embodiments 78-82, wherein the structured motif comprises a nucleic acid sequence selected from any one of SEQ ID NO: 75, 76, 77, 78, 79, 80, 81, or 82.
84. The method of any one of embodiments 1-83, wherein the retron transcript linked to the gRNA molecule comprises a formula A1-Rt-L₁ₙ-gRNA-A2, wherein:
   A1 is the structured motif, or is absent;
   Rt is the retron transcript;
   L₁ₙ is a nucleotide linker from about 5 to about 40, about 8 to about 35, about 9, or about 33 basepairs, or is absent; and
   A2 is the structured motif, or is absent.
85. The method of embodiment 84, wherein:
   A1 is the structured motif;
   Rt is the retron transcript;
   L₁ₙ is a nucleotide linker from about 5 to about 40, about 8 to about 35, about 9, or about 33 basepairs; and
   A2 is absent.
86. The method of embodiment 84, wherein:
A1 is absent;
   Rt is the retron transcript;
   L1 is a nucleotide linker from about 5 to about 40, about 8 to about 35, about 9, or about 33 base pairs; and
A2 is the structured motif.
87. A method of treating a disease or condition by modifying one or more target nucleic acids of interest at one or more target loci within a genome of a host cell, such as a mammalian cells, the method comprising the method of any one of embodiments 1-86.
88. A method for modifying one or more target nucleic acids of interest at one or more target loci within a genome of a host cell, such as a mammalian cells, the method comprising the method of any one of embodiments 1-86.
89. A retron-guide RNA (gRNA) cassette comprising:
a retron comprising:
a first inverted repeat sequence coding region;
a msr locus and a msd locus;
a donor DNA sequence located in the msd locus; and
a second inverted repeat sequence coding region; and
   optionally a gRNA coding region,
wherein the msr locus and the msd locus comprises the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2.
90. The cassette of embodiment 89, wherein the msd locus comprises a deletion of at least 1 nucleotide as compared to SEQ ID NO: 1, or 2.
91. The cassette of embodiment 89, wherein the msd locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to SEQ ID NO: 1, or 2.
92. The cassette of any one of embodiments 89-91, wherein the msd locus upstream (to the 5′ of) of the donor DNA sequence coding region comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18.
93. The cassette of any one of embodiments 89-92, wherein the msd locus upstream (to the 5′ of) of the donor DNA sequence coding region comprises a nucleic acid sequence of any one of SEQ ID NO: 4, 5, 6, 7, 8, 14, 15, 16, 17, or 18.
94. The cassette of any one of embodiments 89-93, wherein the msd locus downstream (to the 3′ of) of the donor DNA sequence coding region comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23.
95. The cassette of any one of embodiments 89-94, wherein the msd locus downstream (to the 3′ of) of the donor DNA sequence coding region comprises a nucleic acid sequence of any one of SEQ ID NO: 9, 10, 11, 12, 13, 19, 20, 21, 22, or 23.
96. The cassette of any one of embodiments 89-95, wherein the retron comprises a nucleic acid molecule comprising:
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18, upstream of the donor DNA sequence coding region coding region; and
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23, downstream of the donor DNA sequence.
97. The cassette of any one of embodiments 89-96, wherein the retron comprises a nucleic acid molecule comprising:
a nucleic acid sequence of any one of SEQ ID NO: 4, 5, 6, 7, 8, 14, 15, 16, 17, or 18, upstream of the donor DNA sequence coding region coding region; and
a nucleic acid sequence of any one of SEQ ID NO: 9, 10, 11, 12, 13, 19, 20, 21, 22, or 23, downstream of the donor DNA sequence.
98. The cassette of any one of embodiments 89-97, wherein the retron comprises a nucleic acid molecule comprising:
a nucleic acid sequence of SEQ ID NO: 4 upstream of the donor DNA sequence; and a nucleic acid sequence of SEQ ID NO: 9 downstream of the donor DNA sequence;
a nucleic acid sequence of SEQ ID NO: 4 upstream of the donor DNA sequence; and a nucleic acid sequence of SEQ ID NO: 10 downstream of the donor DNA sequence;
a nucleic acid sequence of SEQ ID NO: 4 upstream of the donor DNA sequence; and a nucleic acid sequence of SEQ ID NO: 11 downstream of the donor DNA sequence;
a nucleic acid sequence of SEQ ID NO: 4 upstream of the donor DNA sequence; and a nucleic acid sequence of SEQ ID NO: 12 downstream of the donor DNA sequence;
a nucleic acid sequence of SEQ ID NO: 4 upstream of the donor DNA sequence; and a nucleic acid sequence of SEQ ID NO: 13 downstream of the donor DNA sequence;
a nucleic acid sequence of SEQ ID NO: 4 upstream of the donor DNA sequence; and a nucleic acid sequence of SEQ ID NO: 19 downstream of the donor DNA sequence;
a nucleic acid sequence of SEQ ID NO: 4 upstream of the donor DNA sequence; and a nucleic acid sequence of SEQ ID NO: 20 downstream of the donor DNA sequence;
a nucleic acid sequence of SEQ ID NO: 4 upstream of the donor DNA sequence; and a nucleic acid sequence of SEQ ID NO: 21 downstream of the donor DNA sequence;
a nucleic acid sequence of SEQ ID NO: 4 upstream of the donor DNA sequence; and a nucleic acid sequence of SEQ ID NO: 22 downstream of the donor DNA sequence;
a nucleic acid sequence of SEQ ID NO: 4 upstream of the donor DNA sequence; and a nucleic acid sequence of SEQ ID NO: 23 downstream of the donor DNA sequence;
a nucleic acid sequence of SEQ ID NO: 5 upstream of the donor DNA sequence; and a nucleic acid sequence of SEQ ID NO: 9 downstream of the donor DNA sequence;
a nucleic acid sequence of SEQ ID NO: 5 upstream of the donor DNA sequence; and a nucleic acid sequence of SEQ ID NO: 10 downstream of the donor DNA sequence;
a nucleic acid sequence of SEQ ID NO: 5 upstream of the donor DNA sequence; and a nucleic acid sequence of SEQ ID NO: 11 downstream of the donor DNA sequence;
a nucleic acid sequence of SEQ ID NO: 5 upstream of the donor DNA sequence; and a nucleic acid sequence of SEQ ID NO: 12 downstream of the donor DNA sequence;
a nucleic acid sequence of SEQ ID NO: 5 upstream of the donor DNA sequence; and a nucleic acid sequence of SEQ ID NO: 13 downstream of the donor DNA sequence;
a nucleic acid sequence of SEQ ID NO: 5 upstream of the donor DNA sequence; and a nucleic acid sequence of SEQ ID NO: 19 downstream of the donor DNA sequence;
a nucleic acid sequence of SEQ ID NO: 5 upstream of the donor DNA sequence; and a nucleic acid sequence of SEQ ID NO: 20 downstream of the donor DNA sequence;
a nucleic acid sequence of SEQ ID NO: 5 upstream of the donor DNA sequence; and a nucleic acid sequence of SEQ ID NO: 21 downstream of the donor DNA sequence;
a nucleic acid sequence of SEQ ID NO: 5 upstream of the donor DNA sequence; and a nucleic acid sequence of SEQ ID NO: 22 downstream of the donor DNA sequence;
a nucleic acid sequence of SEQ ID NO: 5 upstream of the donor DNA sequence; and a nucleic acid sequence of SEQ ID NO: 23 downstream of the donor DNA sequence;
a nucleic acid sequence of SEQ ID NO: 6 upstream of the donor DNA sequence; and a nucleic acid sequence of SEQ ID NO: 9 downstream of the donor DNA sequence;
a nucleic acid sequence of SEQ ID NO: 6 upstream of the donor DNA sequence; and a nucleic acid sequence of SEQ ID NO: 10 downstream of the donor DNA sequence;
a nucleic acid sequence of SEQ ID NO: 6 upstream of the donor DNA sequence; and a nucleic acid sequence of SEQ ID NO: 11 downstream of the donor DNA sequence;
a nucleic acid sequence of SEQ ID NO: 6 upstream of the donor DNA sequence; and a nucleic acid sequence of SEQ ID NO: 12 downstream of the donor DNA sequence;
a nucleic acid sequence of SEQ ID NO: 6 upstream of the donor DNA sequence; and a nucleic acid sequence of SEQ ID NO: 13 downstream of the donor DNA sequence;
a nucleic acid sequence of SEQ ID NO: 6 upstream of the donor DNA sequence; and a nucleic acid sequence of SEQ ID NO: 19 downstream of the donor DNA sequence;
a nucleic acid sequence of SEQ ID NO: 6 upstream of the donor DNA sequence; and a nucleic acid sequence of SEQ ID NO: 20 downstream of the donor DNA sequence;
a nucleic acid sequence of SEQ ID NO: 6 upstream of the donor DNA sequence; and a nucleic acid sequence of SEQ ID NO: 21 downstream of the donor DNA sequence;
a nucleic acid sequence of SEQ ID NO: 6 upstream of the donor DNA sequence; and a nucleic acid sequence of SEQ ID NO: 22 downstream of the donor DNA sequence;
a nucleic acid sequence of SEQ ID NO: 6 upstream of the donor DNA sequence; and a nucleic acid sequence of SEQ ID NO: 23 downstream of the donor DNA sequence;
a nucleic acid sequence of SEQ ID NO: 7 upstream of the donor DNA sequence; and a nucleic acid sequence of SEQ ID NO: 9 downstream of the donor DNA sequence;
a nucleic acid sequence of SEQ ID NO: 7 upstream of the donor DNA sequence; and a nucleic acid sequence of SEQ ID NO: 10 downstream of the donor DNA sequence;
a nucleic acid sequence of SEQ ID NO: 7 upstream of the donor DNA sequence; and a nucleic acid sequence of SEQ ID NO: 11 downstream of the donor DNA sequence;
a nucleic acid sequence of SEQ ID NO: 7 upstream of the donor DNA sequence; and a nucleic acid sequence of SEQ ID NO: 12 downstream of the donor DNA sequence;
a nucleic acid sequence of SEQ ID NO: 7 upstream of the donor DNA sequence; and a nucleic acid sequence of SEQ ID NO: 13 downstream of the donor DNA sequence;
a nucleic acid sequence of SEQ ID NO: 7 upstream of the donor DNA sequence; and a nucleic acid sequence of SEQ ID NO: 19 downstream of the donor DNA sequence;
a nucleic acid sequence of SEQ ID NO: 7 upstream of the donor DNA sequence; and a nucleic acid sequence of SEQ ID NO: 20 downstream of the donor DNA sequence;
a nucleic acid sequence of SEQ ID NO: 7 upstream of the donor DNA sequence; and a nucleic acid sequence of SEQ ID NO: 21 downstream of the donor DNA sequence;
a nucleic acid sequence of SEQ ID NO: 7 upstream of the donor DNA sequence; and a nucleic acid sequence of SEQ ID NO: 22 downstream of the donor DNA sequence;
a nucleic acid sequence of SEQ ID NO: 7 upstream of the donor DNA sequence; and a nucleic acid sequence of SEQ ID NO: 23 downstream of the donor DNA sequence;
a nucleic acid sequence of SEQ ID NO: 8 upstream of the donor DNA sequence; and a nucleic acid sequence of SEQ ID NO: 9 downstream of the donor DNA sequence;
a nucleic acid sequence of SEQ ID NO: 8 upstream of the donor DNA sequence; and a nucleic acid sequence of SEQ ID NO: 10 downstream of the donor DNA sequence;
a nucleic acid sequence of SEQ ID NO: 8 upstream of the donor DNA sequence; and a nucleic acid sequence of SEQ ID NO: 11 downstream of the donor DNA sequence;
a nucleic acid sequence of SEQ ID NO: 8 upstream of the donor DNA sequence; and a nucleic acid sequence of SEQ ID NO: 12 downstream of the donor DNA sequence;
a nucleic acid sequence of SEQ ID NO: 8 upstream of the donor DNA sequence; and a nucleic acid sequence of SEQ ID NO: 13 downstream of the donor DNA sequence;
a nucleic acid sequence of SEQ ID NO: 8 upstream of the donor DNA sequence; and a nucleic acid sequence of SEQ ID NO: 19 downstream of the donor DNA sequence;
a nucleic acid sequence of SEQ ID NO: 8 upstream of the donor DNA sequence; and a nucleic acid sequence of SEQ ID NO: 20 downstream of the donor DNA sequence;
a nucleic acid sequence of SEQ ID NO: 8 upstream of the donor DNA sequence; and a nucleic acid sequence of SEQ ID NO: 21 downstream of the donor DNA sequence;
a nucleic acid sequence of SEQ ID NO: 8 upstream of the donor DNA sequence; and a nucleic acid sequence of SEQ ID NO: 22 downstream of the donor DNA sequence;
a nucleic acid sequence of SEQ ID NO: 8 upstream of the donor DNA sequence; and a nucleic acid sequence of SEQ ID NO: 23 downstream of the donor DNA sequence;
a nucleic acid sequence of SEQ ID NO: 14 upstream of the donor DNA sequence; and a nucleic acid sequence of SEQ ID NO: 9 downstream of the donor DNA sequence;
a nucleic acid sequence of SEQ ID NO: 14 upstream of the donor DNA sequence; and a nucleic acid sequence of SEQ ID NO: 10 downstream of the donor DNA sequence;
a nucleic acid sequence of SEQ ID NO: 14 upstream of the donor DNA sequence; and a nucleic acid sequence of SEQ ID NO: 11 downstream of the donor DNA sequence;
a nucleic acid sequence of SEQ ID NO: 14 upstream of the donor DNA sequence; and a nucleic acid sequence of SEQ ID NO: 12 downstream of the donor DNA sequence;
a nucleic acid sequence of SEQ ID NO: 14 upstream of the donor DNA sequence; and a nucleic acid sequence of SEQ ID NO: 13 downstream of the donor DNA sequence;
a nucleic acid sequence of SEQ ID NO: 14 upstream of the donor DNA sequence; and a nucleic acid sequence of SEQ ID NO: 19 downstream of the donor DNA sequence;
a nucleic acid sequence of SEQ ID NO: 14 upstream of the donor DNA sequence; and a nucleic acid sequence of SEQ ID NO: 20 downstream of the donor DNA sequence;
a nucleic acid sequence of SEQ ID NO: 14 upstream of the donor DNA sequence; and a nucleic acid sequence of SEQ ID NO: 21 downstream of the donor DNA sequence;
a nucleic acid sequence of SEQ ID NO: 14 upstream of the donor DNA sequence; and a nucleic acid sequence of SEQ ID NO: 22 downstream of the donor DNA sequence;
a nucleic acid sequence of SEQ ID NO: 14 upstream of the donor DNA sequence; and a nucleic acid sequence of SEQ ID NO: 23 downstream of the donor DNA sequence;
a nucleic acid sequence of SEQ ID NO: 15 upstream of the donor DNA sequence; and a nucleic acid sequence of SEQ ID NO: 9 downstream of the donor DNA sequence;
a nucleic acid sequence of SEQ ID NO: 15 upstream of the donor DNA sequence; and a nucleic acid sequence of SEQ ID NO: 10 downstream of the donor DNA sequence;
a nucleic acid sequence of SEQ ID NO: 15 upstream of the donor DNA sequence; and a nucleic acid sequence of SEQ ID NO: 11 downstream of the donor DNA sequence;
a nucleic acid sequence of SEQ ID NO: 15 upstream of the donor DNA sequence; and a nucleic acid sequence of SEQ ID NO: 12 downstream of the donor DNA sequence;
a nucleic acid sequence of SEQ ID NO: 15 upstream of the donor DNA sequence; and a nucleic acid sequence of SEQ ID NO: 13 downstream of the donor DNA sequence;
a nucleic acid sequence of SEQ ID NO: 15 upstream of the donor DNA sequence; and a nucleic acid sequence of SEQ ID NO: 19 downstream of the donor DNA sequence;
a nucleic acid sequence of SEQ ID NO: 15 upstream of the donor DNA sequence; and a nucleic acid sequence of SEQ ID NO: 20 downstream of the donor DNA sequence;
a nucleic acid sequence of SEQ ID NO: 15 upstream of the donor DNA sequence; and a nucleic acid sequence of SEQ ID NO: 21 downstream of the donor DNA sequence;
a nucleic acid sequence of SEQ ID NO: 15 upstream of the donor DNA sequence; and a nucleic acid sequence of SEQ ID NO: 22 downstream of the donor DNA sequence;
a nucleic acid sequence of SEQ ID NO: 15 upstream of the donor DNA sequence; and a nucleic acid sequence of SEQ ID NO: 23 downstream of the donor DNA sequence;
a nucleic acid sequence of SEQ ID NO: 16 upstream of the donor DNA sequence; and a nucleic acid sequence of SEQ ID NO: 9 downstream of the donor DNA sequence;
a nucleic acid sequence of SEQ ID NO: 16 upstream of the donor DNA sequence; and a nucleic acid sequence of SEQ ID NO: 10 downstream of the donor DNA sequence;
a nucleic acid sequence of SEQ ID NO: 16 upstream of the donor DNA sequence; and a nucleic acid sequence of SEQ ID NO: 11 downstream of the donor DNA sequence;
a nucleic acid sequence of SEQ ID NO: 16 upstream of the donor DNA sequence; and a nucleic acid sequence of SEQ ID NO: 12 downstream of the donor DNA sequence;
a nucleic acid sequence of SEQ ID NO: 16 upstream of the donor DNA sequence; and a nucleic acid sequence of SEQ ID NO: 13 downstream of the donor DNA sequence;
a nucleic acid sequence of SEQ ID NO: 16 upstream of the donor DNA sequence; and a nucleic acid sequence of SEQ ID NO: 19 downstream of the donor DNA sequence;
a nucleic acid sequence of SEQ ID NO: 16 upstream of the donor DNA sequence; and a nucleic acid sequence of SEQ ID NO: 20 downstream of the donor DNA sequence;
a nucleic acid sequence of SEQ ID NO: 16 upstream of the donor DNA sequence; and a nucleic acid sequence of SEQ ID NO: 21 downstream of the donor DNA sequence;
a nucleic acid sequence of SEQ ID NO: 16 upstream of the donor DNA sequence; and a nucleic acid sequence of SEQ ID NO: 22 downstream of the donor DNA sequence;
a nucleic acid sequence of SEQ ID NO: 16 upstream of the donor DNA sequence; and a nucleic acid sequence of SEQ ID NO: 23 downstream of the donor DNA sequence;
a nucleic acid sequence of SEQ ID NO: 17 upstream of the donor DNA sequence; and a nucleic acid sequence of SEQ ID NO: 9 downstream of the donor DNA sequence;
a nucleic acid sequence of SEQ ID NO: 17 upstream of the donor DNA sequence; and a nucleic acid sequence of SEQ ID NO: 10 downstream of the donor DNA sequence;
a nucleic acid sequence of SEQ ID NO: 17 upstream of the donor DNA sequence; and a nucleic acid sequence of SEQ ID NO: 11 downstream of the donor DNA sequence;
a nucleic acid sequence of SEQ ID NO: 17 upstream of the donor DNA sequence; and a nucleic acid sequence of SEQ ID NO: 12 downstream of the donor DNA sequence;
a nucleic acid sequence of SEQ ID NO: 17 upstream of the donor DNA sequence; and a nucleic acid sequence of SEQ ID NO: 13 downstream of the donor DNA sequence;
a nucleic acid sequence of SEQ ID NO: 17 upstream of the donor DNA sequence; and a nucleic acid sequence of SEQ ID NO: 19 downstream of the donor DNA sequence;
a nucleic acid sequence of SEQ ID NO: 17 upstream of the donor DNA sequence; and a nucleic acid sequence of SEQ ID NO: 20 downstream of the donor DNA sequence;
a nucleic acid sequence of SEQ ID NO: 17 upstream of the donor DNA sequence; and a nucleic acid sequence of SEQ ID NO: 21 downstream of the donor DNA sequence;
a nucleic acid sequence of SEQ ID NO: 17 upstream of the donor DNA sequence; and a nucleic acid sequence of SEQ ID NO: 22 downstream of the donor DNA sequence;
a nucleic acid sequence of SEQ ID NO: 17 upstream of the donor DNA sequence; and a nucleic acid sequence of SEQ ID NO: 23 downstream of the donor DNA sequence;
a nucleic acid sequence of SEQ ID NO: 18 upstream of the donor DNA sequence; and a nucleic acid sequence of SEQ ID NO: 9 downstream of the donor DNA sequence;
a nucleic acid sequence of SEQ ID NO: 18 upstream of the donor DNA sequence; and a nucleic acid sequence of SEQ ID NO: 10 downstream of the donor DNA sequence;
a nucleic acid sequence of SEQ ID NO: 18 upstream of the donor DNA sequence; and a nucleic acid sequence of SEQ ID NO: 11 downstream of the donor DNA sequence;
a nucleic acid sequence of SEQ ID NO: 18 upstream of the donor DNA sequence; and a nucleic acid sequence of SEQ ID NO: 12 downstream of the donor DNA sequence;
a nucleic acid sequence of SEQ ID NO: 18 upstream of the donor DNA sequence; and a nucleic acid sequence of SEQ ID NO: 13 downstream of the donor DNA sequence;
a nucleic acid sequence of SEQ ID NO: 18 upstream of the donor DNA sequence; and a nucleic acid sequence of SEQ ID NO: 19 downstream of the donor DNA sequence;
a nucleic acid sequence of SEQ ID NO: 18 upstream of the donor DNA sequence; and a nucleic acid sequence of SEQ ID NO: 20 downstream of the donor DNA sequence;
a nucleic acid sequence of SEQ ID NO: 18 upstream of the donor DNA sequence; and a nucleic acid sequence of SEQ ID NO: 21 downstream of the donor DNA sequence;
a nucleic acid sequence of SEQ ID NO: 18 upstream of the donor DNA sequence; and a nucleic acid sequence of SEQ ID NO: 22 downstream of the donor DNA sequence; or a nucleic acid sequence of SEQ ID NO: 18 upstream of the donor DNA sequence; and a nucleic acid sequence of SEQ ID NO: 23 downstream of the donor DNA sequence.
99. The cassette of embodiment 89, wherein the retron comprises a nucleic acid molecule having a formula of 5′-M1-X1-M2-3′, wherein M1 is a fragment of the msr locus and the msd locus, X1 is the donor DNA sequence; and M2 is a fragment of the msd locus and the msr locus.
100. The cassette of embodiment 99, wherein M1 comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33.
101. The cassette of embodiment 100, wherein M1 comprises a nucleic acid sequence of any one of SEQ ID NO: 24, 25, 26, 27, 28, 29, 30, 31, 32, or 33.
102. The cassette of embodiment 99, wherein M2 comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.
103. The cassette of embodiment 102, wherein M2 comprises a nucleic acid sequence of any one of SEQ ID NO: 34, 35, 36, 37, 38, 39, 40, 41, 42, or 43.
104. The cassette of any one of embodiments 99-103, wherein the nucleic acid molecule comprises an M1 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and an M2 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.
105. The cassette of embodiment 99, wherein the nucleic acid molecule comprises an M1 having a nucleic acid sequence of SEQ ID NO: 24; and an M2 having a nucleic acid sequence of SEQ ID NO: 34;
an M1 having a nucleic acid sequence of SEQ ID NO: 24; and an M2 having a nucleic acid sequence of SEQ ID NO: 35;
an M1 having a nucleic acid sequence of SEQ ID NO: 24; and an M2 having a nucleic acid sequence of SEQ ID NO: 36;
an M1 having a nucleic acid sequence of SEQ ID NO: 24; and an M2 having a nucleic acid sequence of SEQ ID NO: 37;
an M1 having a nucleic acid sequence of SEQ ID NO: 24; and an M2 having a nucleic acid sequence of SEQ ID NO: 38;
an M1 having a nucleic acid sequence of SEQ ID NO: 24; and an M2 having a nucleic acid sequence of SEQ ID NO: 39;
an M1 having a nucleic acid sequence of SEQ ID NO: 24; and an M2 having a nucleic acid sequence of SEQ ID NO: 40;
an M1 having a nucleic acid sequence of SEQ ID NO: 24; and an M2 having a nucleic acid sequence of SEQ ID NO: 41;
an M1 having a nucleic acid sequence of SEQ ID NO: 24; and an M2 having a nucleic acid sequence of SEQ ID NO: 42;
an M1 having a nucleic acid sequence of SEQ ID NO: 24; and an M2 having a nucleic acid sequence of SEQ ID NO: 43;
an M1 having a nucleic acid sequence of SEQ ID NO: 25; and an M2 having a nucleic acid sequence of SEQ ID NO: 34;
an M1 having a nucleic acid sequence of SEQ ID NO: 25; and an M2 having a nucleic acid sequence of SEQ ID NO: 35;
an M1 having a nucleic acid sequence of SEQ ID NO: 25; and an M2 having a nucleic acid sequence of SEQ ID NO: 36;
an M1 having a nucleic acid sequence of SEQ ID NO: 25; and an M2 having a nucleic acid sequence of SEQ ID NO: 37;
an M1 having a nucleic acid sequence of SEQ ID NO: 25; and an M2 having a nucleic acid sequence of SEQ ID NO: 38;
an M1 having a nucleic acid sequence of SEQ ID NO: 25; and an M2 having a nucleic acid sequence of SEQ ID NO: 39;
an M1 having a nucleic acid sequence of SEQ ID NO: 25; and an M2 having a nucleic acid sequence of SEQ ID NO: 40;
an M1 having a nucleic acid sequence of SEQ ID NO: 25; and an M2 having a nucleic acid sequence of SEQ ID NO: 41;
an M1 having a nucleic acid sequence of SEQ ID NO: 25; and an M2 having a nucleic acid sequence of SEQ ID NO: 42;
an M1 having a nucleic acid sequence of SEQ ID NO: 25; and an M2 having a nucleic acid sequence of SEQ ID NO: 43;
an M1 having a nucleic acid sequence of SEQ ID NO: 26; and an M2 having a nucleic acid sequence of SEQ ID NO: 34;
an M1 having a nucleic acid sequence of SEQ ID NO: 26; and an M2 having a nucleic acid sequence of SEQ ID NO: 35;
an M1 having a nucleic acid sequence of SEQ ID NO: 26; and an M2 having a nucleic acid sequence of SEQ ID NO: 36;
an M1 having a nucleic acid sequence of SEQ ID NO: 26; and an M2 having a nucleic acid sequence of SEQ ID NO: 37;
an M1 having a nucleic acid sequence of SEQ ID NO: 26; and an M2 having a nucleic acid sequence of SEQ ID NO: 38;
an M1 having a nucleic acid sequence of SEQ ID NO: 26; and an M2 having a nucleic acid sequence of SEQ ID NO: 39;
an M1 having a nucleic acid sequence of SEQ ID NO: 26; and an M2 having a nucleic acid sequence of SEQ ID NO: 40;
an M1 having a nucleic acid sequence of SEQ ID NO: 26; and an M2 having a nucleic acid sequence of SEQ ID NO: 41;
an M1 having a nucleic acid sequence of SEQ ID NO: 26; and an M2 having a nucleic acid sequence of SEQ ID NO: 42;
an M1 having a nucleic acid sequence of SEQ ID NO: 26; and an M2 having a nucleic acid sequence of SEQ ID NO: 43;
an M1 having a nucleic acid sequence of SEQ ID NO: 27; and an M2 having a nucleic acid sequence of SEQ ID NO: 34;
an M1 having a nucleic acid sequence of SEQ ID NO: 27; and an M2 having a nucleic acid sequence of SEQ ID NO: 35;
an M1 having a nucleic acid sequence of SEQ ID NO: 27; and an M2 having a nucleic acid sequence of SEQ ID NO: 36;
an M1 having a nucleic acid sequence of SEQ ID NO: 27; and an M2 having a nucleic acid sequence of SEQ ID NO: 37;
an M1 having a nucleic acid sequence of SEQ ID NO: 27; and an M2 having a nucleic acid sequence of SEQ ID NO: 38;
an M1 having a nucleic acid sequence of SEQ ID NO: 27; and an M2 having a nucleic acid sequence of SEQ ID NO: 39;
an M1 having a nucleic acid sequence of SEQ ID NO: 27; and an M2 having a nucleic acid sequence of SEQ ID NO: 40;
an M1 having a nucleic acid sequence of SEQ ID NO: 27; and an M2 having a nucleic acid sequence of SEQ ID NO: 41;
an M1 having a nucleic acid sequence of SEQ ID NO: 27; and an M2 having a nucleic acid sequence of SEQ ID NO: 42;
an M1 having a nucleic acid sequence of SEQ ID NO: 27; and an M2 having a nucleic acid sequence of SEQ ID NO: 43;
an M1 having a nucleic acid sequence of SEQ ID NO: 28; and an M2 having a nucleic acid sequence of SEQ ID NO: 34;
an M1 having a nucleic acid sequence of SEQ ID NO: 28; and an M2 having a nucleic acid sequence of SEQ ID NO: 35;
an M1 having a nucleic acid sequence of SEQ ID NO: 28; and an M2 having a nucleic acid sequence of SEQ ID NO: 36;
an M1 having a nucleic acid sequence of SEQ ID NO: 28; and an M2 having a nucleic acid sequence of SEQ ID NO: 37;
an M1 having a nucleic acid sequence of SEQ ID NO: 28; and an M2 having a nucleic acid sequence of SEQ ID NO: 38;
an M1 having a nucleic acid sequence of SEQ ID NO: 28; and an M2 having a nucleic acid sequence of SEQ ID NO: 39;
an M1 having a nucleic acid sequence of SEQ ID NO: 28; and an M2 having a nucleic acid sequence of SEQ ID NO: 40;
an M1 having a nucleic acid sequence of SEQ ID NO: 28; and an M2 having a nucleic acid sequence of SEQ ID NO: 41;
an M1 having a nucleic acid sequence of SEQ ID NO: 28; and an M2 having a nucleic acid sequence of SEQ ID NO: 42;
an M1 having a nucleic acid sequence of SEQ ID NO: 28; and an M2 having a nucleic acid sequence of SEQ ID NO: 43;
an M1 having a nucleic acid sequence of SEQ ID NO: 29; and an M2 having a nucleic acid sequence of SEQ ID NO: 34;
an M1 having a nucleic acid sequence of SEQ ID NO: 29; and an M2 having a nucleic acid sequence of SEQ ID NO: 35;
an M1 having a nucleic acid sequence of SEQ ID NO: 29; and an M2 having a nucleic acid sequence of SEQ ID NO: 36;
an M1 having a nucleic acid sequence of SEQ ID NO: 29; and an M2 having a nucleic acid sequence of SEQ ID NO: 37;
an M1 having a nucleic acid sequence of SEQ ID NO: 29; and an M2 having a nucleic acid sequence of SEQ ID NO: 38;
an M1 having a nucleic acid sequence of SEQ ID NO: 29; and an M2 having a nucleic acid sequence of SEQ ID NO: 39;
an M1 having a nucleic acid sequence of SEQ ID NO: 29; and an M2 having a nucleic acid sequence of SEQ ID NO: 40;
an M1 having a nucleic acid sequence of SEQ ID NO: 29; and an M2 having a nucleic acid sequence of SEQ ID NO: 41;
an M1 having a nucleic acid sequence of SEQ ID NO: 29; and an M2 having a nucleic acid sequence of SEQ ID NO: 42;
an M1 having a nucleic acid sequence of SEQ ID NO: 29; and an M2 having a nucleic acid sequence of SEQ ID NO: 43;
an M1 having a nucleic acid sequence of SEQ ID NO: 30; and an M2 having a nucleic acid sequence of SEQ ID NO: 34;
an M1 having a nucleic acid sequence of SEQ ID NO: 30; and an M2 having a nucleic acid sequence of SEQ ID NO: 35;
an M1 having a nucleic acid sequence of SEQ ID NO: 30; and an M2 having a nucleic acid sequence of SEQ ID NO: 36;
an M1 having a nucleic acid sequence of SEQ ID NO: 30; and an M2 having a nucleic acid sequence of SEQ ID NO: 37;
an M1 having a nucleic acid sequence of SEQ ID NO: 30; and an M2 having a nucleic acid sequence of SEQ ID NO: 38;
an M1 having a nucleic acid sequence of SEQ ID NO: 30; and an M2 having a nucleic acid sequence of SEQ ID NO: 39;
an M1 having a nucleic acid sequence of SEQ ID NO: 30; and an M2 having a nucleic acid sequence of SEQ ID NO: 40;
an M1 having a nucleic acid sequence of SEQ ID NO: 30; and an M2 having a nucleic acid sequence of SEQ ID NO: 41;
an M1 having a nucleic acid sequence of SEQ ID NO: 30; and an M2 having a nucleic acid sequence of SEQ ID NO: 42;
an M1 having a nucleic acid sequence of SEQ ID NO: 30; and an M2 having a nucleic acid sequence of SEQ ID NO: 43;
an M1 having a nucleic acid sequence of SEQ ID NO: 31; and an M2 having a nucleic acid sequence of SEQ ID NO: 34;
an M1 having a nucleic acid sequence of SEQ ID NO: 31; and an M2 having a nucleic acid sequence of SEQ ID NO: 35;
an M1 having a nucleic acid sequence of SEQ ID NO: 31; and an M2 having a nucleic acid sequence of SEQ ID NO: 36;
an M1 having a nucleic acid sequence of SEQ ID NO: 31; and an M2 having a nucleic acid sequence of SEQ ID NO: 37;
an M1 having a nucleic acid sequence of SEQ ID NO: 31; and an M2 having a nucleic acid sequence of SEQ ID NO: 38;
an M1 having a nucleic acid sequence of SEQ ID NO: 31; and an M2 having a nucleic acid sequence of SEQ ID NO: 39;
an M1 having a nucleic acid sequence of SEQ ID NO: 31; and an M2 having a nucleic acid sequence of SEQ ID NO: 40;
an M1 having a nucleic acid sequence of SEQ ID NO: 31; and an M2 having a nucleic acid sequence of SEQ ID NO: 41;
an M1 having a nucleic acid sequence of SEQ ID NO: 31; and an M2 having a nucleic acid sequence of SEQ ID NO: 42;
an M1 having a nucleic acid sequence of SEQ ID NO: 31; and an M2 having a nucleic acid sequence of SEQ ID NO: 43;
an M1 having a nucleic acid sequence of SEQ ID NO: 32; and an M2 having a nucleic acid sequence of SEQ ID NO: 34;
an M1 having a nucleic acid sequence of SEQ ID NO: 32; and an M2 having a nucleic acid sequence of SEQ ID NO: 35;
an M1 having a nucleic acid sequence of SEQ ID NO: 32; and an M2 having a nucleic acid sequence of SEQ ID NO: 36;
an M1 having a nucleic acid sequence of SEQ ID NO: 32; and an M2 having a nucleic acid sequence of SEQ ID NO: 37;
an M1 having a nucleic acid sequence of SEQ ID NO: 32; and an M2 having a nucleic acid sequence of SEQ ID NO: 38;
an M1 having a nucleic acid sequence of SEQ ID NO: 32; and an M2 having a nucleic acid sequence of SEQ ID NO: 39;
an M1 having a nucleic acid sequence of SEQ ID NO: 32; and an M2 having a nucleic acid sequence of SEQ ID NO: 40;
an M1 having a nucleic acid sequence of SEQ ID NO: 32; and an M2 having a nucleic acid sequence of SEQ ID NO: 41;
an M1 having a nucleic acid sequence of SEQ ID NO: 32; and an M2 having a nucleic acid sequence of SEQ ID NO: 42;
an M1 having a nucleic acid sequence of SEQ ID NO: 32; and an M2 having a nucleic acid sequence of SEQ ID NO: 43;
an M1 having a nucleic acid sequence of SEQ ID NO: 33; and an M2 having a nucleic acid sequence of SEQ ID NO: 34;
an M1 having a nucleic acid sequence of SEQ ID NO: 33; and an M2 having a nucleic acid sequence of SEQ ID NO: 35;
an M1 having a nucleic acid sequence of SEQ ID NO: 33; and an M2 having a nucleic acid sequence of SEQ ID NO: 36;
an M1 having a nucleic acid sequence of SEQ ID NO: 33; and an M2 having a nucleic acid sequence of SEQ ID NO: 37;
an M1 having a nucleic acid sequence of SEQ ID NO: 33; and an M2 having a nucleic acid sequence of SEQ ID NO: 38;
an M1 having a nucleic acid sequence of SEQ ID NO: 33; and an M2 having a nucleic acid sequence of SEQ ID NO: 39;
an M1 having a nucleic acid sequence of SEQ ID NO: 33; and an M2 having a nucleic acid sequence of SEQ ID NO: 40;
an M1 having a nucleic acid sequence of SEQ ID NO: 33; and an M2 having a nucleic acid sequence of SEQ ID NO: 41;
an M1 having a nucleic acid sequence of SEQ ID NO: 33; and an M2 having a nucleic acid sequence of SEQ ID NO: 42; or
   an M1 having a nucleic acid sequence of SEQ ID NO: 33; and an M2 having a nucleic acid sequence of SEQ ID NO: 43.
106. The cassette of any one of embodiments 89-105, wherein the donor DNA sequence is target strand donor DNA sequence.
107. A retron donor DNA-guide polynucleotide comprising a retron transcript comprising the product of the cassette of any one of embodiments 89-105, and optionally a gRNA.
108. The polynucleotide of embodiment 107, wherein the retron transcript and the gRNA are not linked together.
109. The polynucleotide of embodiment 107, wherein the retron transcript and the gRNA are linked together.
110. The polynucleotide of embodiment 109, wherein the retron transcript and the gRNA are linked together as part of the same transcript.
111. The polynucleotide of embodiment 109, wherein the retron transcript and the gRNA are linked together as not part of the same contiguous transcript.
112. The polynucleotide of embodiment 109, wherein the retron and gRNA are covalently linked together.
113. The polynucleotide of embodiments 109-112, wherein the retron transcript and gRNA are linked together with a linker.
114. The polynucleotide of embodiment 113, wherein the linker is a peptide linker.
115. The polynucleotide of embodiment 114, wherein the peptide linker comprises an amino acid sequence of (GAA)n, wherein n is 1-20, 1-15, 1-10, 1-5, or 1-3.
116. The polynucleotide of any one of embodiments 107-115, wherein the retron transcript and the gRNA comprises a structured motif at a 5′ or a 3′ end of the retron-gRNA transcript.
117. The polynucleotide of embodiment 116, wherein the structured motif is at the 5′ end of the retron-gRNA transcript.
118. The polynucleotide of any one of embodiments 116-117, wherein the structured motif is at the 3′ end of the retron-gRNA transcript.
119. The polynucleotide of any one of embodiments 116-118, wherein the structured motif stabilizes the transcript.
120. The polynucleotide of any one of embodiments 116-119, wherein the structured motif is, independently, evoPreQ, tevopreQ mpKnot, or exoribonuclease-resistant RNA motif.
121. The polynucleotide of any one of embodiments 116-120, wherein the structured motif comprises a nucleic acid sequence selected from any one of SEQ ID NO: 75, 76, 77, 78, 79, 80, 81, or 82.
122. The polynucleotide of any one of embodiments 107-121, wherein the retron transcript linked to the gRNA molecule comprises a formula A1-Rt-L₁ₙ-gRNA-A2, wherein:
   A1 is the structured motif, or is absent;
   Rt is the retron transcript;
   L₁ₙ is a nucleotide linker from about 5 to about 40, about 8 to about 35, about 9, or about 33 basepairs, or is absent; and
   A2 is the structured motif, or is absent.
123. The polynucleotide of embodiment 122, wherein:
   A1 is the structured motif;
   Rt is the retron transcript;
   L₁ₙ is a nucleotide linker from about 5 to about 40, about 8 to about 35, about 9, or about 33 basepairs; and
   A2 is absent.
124. The polynucleotide of embodiment 122, wherein:
A1 is absent;
   Rt is the retron transcript;
   L1 is a nucleotide linker from about 5 to about 40, about 8 to about 35, about 9, or about 33 base pairs; and
A2 is the structured motif.
125. A vector (e.g., plasmid, virus, and the like) comprising the cassette of any one of embodiments 89-106, or the polynucleotide of any one of embodiment 107-124.
126. A polypeptide having the formula of: of (N₁)_{q}-C₁-L₁-(N₂)_{qq}-R₁-(N₃)_{qqq}, wherein C1 is a nuclease, L1 is a peptide linker, R1 is a RT protein, and N1 and N2 are each independently, a NLS sequence, and wherein q, qq, or qqq, are each independently, 0, 1, 2, or 3.
127. The polypeptide of embodiment 126, wherein the qq of 1 comprises the NLS sequence linked at the C-terminus end of the NLS sequence to the N-terminus to R1.
128. The polypeptide of embodiment 126, wherein the q of 2 comprises a first NLS sequence linked to a second NLS sequence with a linker.
129. The polypeptide of any one of embodiments 126-128, wherein the first NLS sequence is identical to the second NLS sequence.
130. The polypeptide of any one of embodiments 126-128, wherein the first NLS sequence is different than the second NLS sequence.
131. The polypeptide of any one of embodiments 126-130, wherein the polypeptide has the formula of:

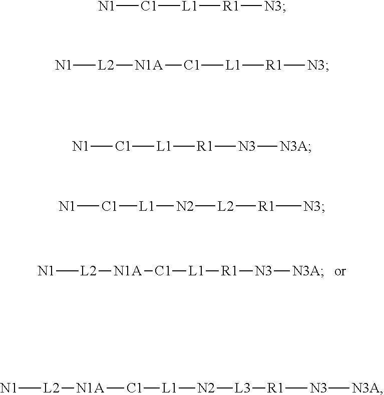

wherein:
each N1, N1A, N2, N3, or N3A is, independently, a NLS sequence;
each L1, L2, or L3 is independently, a linker amino acid sequence; and
wherein each NLS sequence is identical or different.
132. The polypeptide of any one of embodiments 126-131, wherein N1, N1A, N2, N3, or N3A, each independently comprise a SV40 NLS sequence, a cMyc NLS sequence, or a Nuceloplasmin NLS sequence.
133. The polypeptide of any one of embodiments 126-132, wherein the N1 or the N1A comprises the cMyc NLS sequence or the SV40 NLS sequence.
134. The polypeptide of any one of embodiments 126-133, wherein the N2 comprises the SV40 NLS sequence or the Nuceloplasmin NLS sequence.
135. The polypeptide of any one of embodiments 126-134, wherein the N3 or the N3A comprises the Nuceloplasmin NLS sequence.
136. The polypeptide of any one of embodiments 126-135, wherein the linker amino acid sequence each independently comprise an XTEN linker, a G/S linker, a G/A linker, or any combination thereof.
137. The polypeptide of any one of embodiments 126-136, wherein the Cas comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 69.
138. The polypeptide of any one of embodiments 126-137, wherein the Cas comprises an amino acid sequence of SEQ ID NO: 69.
139. The polypeptide of any one of embodiments 126-138, wherein R1 comprises a RT sequence Ec86, Ec48, Ec73, Ec107, or Sa163.
140. The polypeptide of any one of embodiments 126-139, wherein the polypeptide comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74.
141. The polypeptide of any one of embodiments 126-140, wherein the polypeptide comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74, provided that the polypeptide comprises 1, 2, 3, or 4 NLS amino acid sequences.
142. The polypeptide of any one of embodiments 126-141, wherein the polypeptide comprises an amino acid sequence of any one of SEQ ID NO: 70, 71, 72, 73, or 74.
143. The polypeptide of embodiment 142, wherein the polypeptide comprises a domain that has an RT activity and a nuclease activity.
144. A polypeptide comprising a SSAP, RT, and a nuclease, wherein:
the C-terminus of the RT is linked to the N-terminus of the nuclease, and the C-terminus of the nuclease is linked to the N-terminus of the SSAP; or
the N-terminus of the RT is linked to the C-terminus of the nuclease, and the N-terminus of the nuclease is linked to the C-terminus of the SSAP.
145. The polypeptide of embodiment 144, wherein the nuclease is a Cas nuclease.
146. The polypeptide of embodiment 145, wherein the Cas nuclease is a catalytically active nuclease, a nickase Cas nuclease, or a catalytically inactive Cas nuclease.
147. The polypeptide of any one of embodiments 145-146, wherein the Cas nuclease is a Cas9 nuclease, nickase Cas9 nuclease, or a catalytically inactive Cas9 nuclease.
148. The polypeptide of embodiment 147, wherein the nickase Cas9 nuclease is Cas9D10A or Cas9H840A nuclease or a Cas having the both D10A and H840A mutations.
149. A nucleic acid molecule encoding the polypeptide of any one of embodiments 126-148.
150. A vector comprising the nucleic acid molecule of embodiment 149, wherein the vector is a plasmid, virus, RNA, mRNA, and the like.
151. A cell comprising the cassette of any one of embodiments 89-106, the polynucleotide of any one of embodiment 107-124, or the polypeptide of any one of embodiment 126-148.
152. The cell of embodiment 151, wherein the cell is a mammalian cell, such as a human cell.
153. The cell of embodiments 151 or 152, wherein the is cell is in a subject.

The following examples are illustrative, but not limiting, of the compounds, compositions and methods described herein. Other suitable modifications and adaptations known to those skilled in the art are within the scope of the following embodiments.

Example 1: HEK293T cells were transiently transfected with 1) plasmid expressing Cas9 fused to a retron RT (Sa163) and 2) a plasmid expressing a Sa163 retron guide RNA targeting the HEK3 locus with the gRNA at the 5′ or 3′ end. Genomic DNA was harvested 3 days post-transfection and analyzed by next-generation sequencing. As shown in FIG. 2, the downstream or 3′ orientation of the gRNA produced significantly higher editing in these mammalian cells, although both orientations of the gRNA result in editing. Specifically, it was found that positioning of gRNA in retron/gRNA at the 3′ end (downstream) increased HDR rates about 4× compared to 5′ positioning.

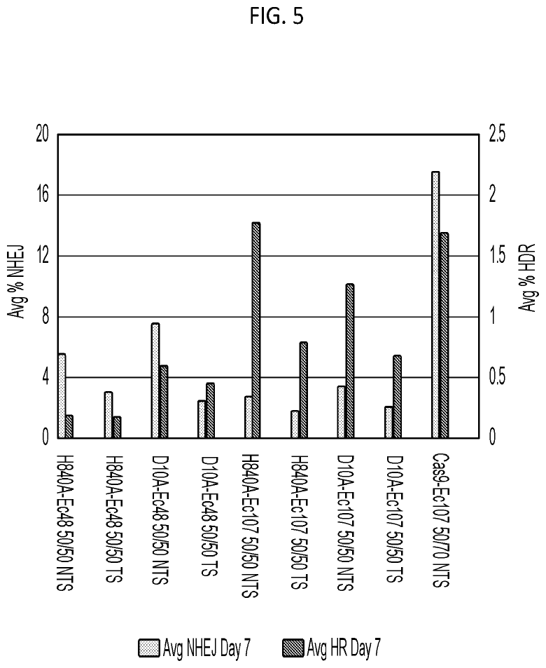

Example 2: The ability of nickase mutants of Cas to participate in retron-mediated editing was examined. HEK293T cells expressing a BFP reporter were used for this experiment. The BFP-to-GFP conversion reporter functions as an editing readout: if precise repair occurs, cells will gain expression of GFP (GFP positive). If cells undergo imprecise repair, cells lose BFP expression and do not gain GFP expression (GFP negative; BFP negative). HEK293T BFP reporter cells were transiently transfected with 1) plasmid expressing Cas9 nickases (Cas9H840A or Cas9D10A) or Cas9WT fused to a retron RT (Ec48 or Ec107) and 2) plasmid expressing a retron guide-RNA with varying msDNA templates with differing degrees of homology. Cells were analyzed 7 days post-transfection on a flow cytometer. The results shown in FIG. 5 indicates that nickase variants of Cas can participate in retron-mediated repair.

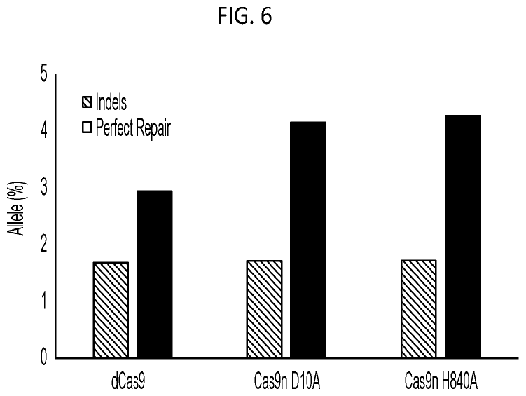

Example 3: Utilizing nickase or dead versions of Cas9 with retron encoded DNA and a single-stranded annealing protein (SSAP) to stimulate gene repair through targeted recombineering or HDR. Plasmid expressing retron (msr/msd) RNA (without CRISPR gRNA) with another plasmid expressing SSAP-dCas9-RT fusion were co-transfected into HEK293T cells. The compositions were found to be effective in editing the genome of the cell. This experiment demonstrates that an active form of Cas9 was not required (FIG. 6); i.e., a dead Cas9 along with SSAP can stimulate gene repair with no DNA cutting.

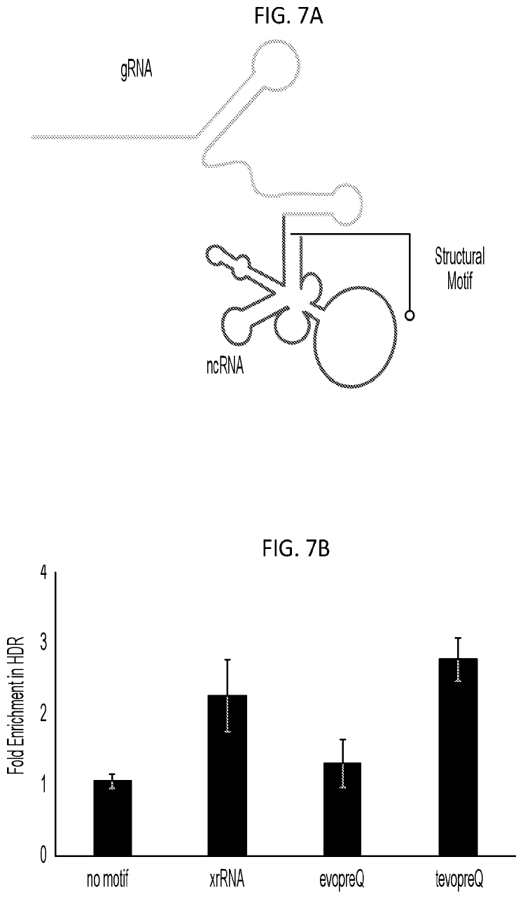

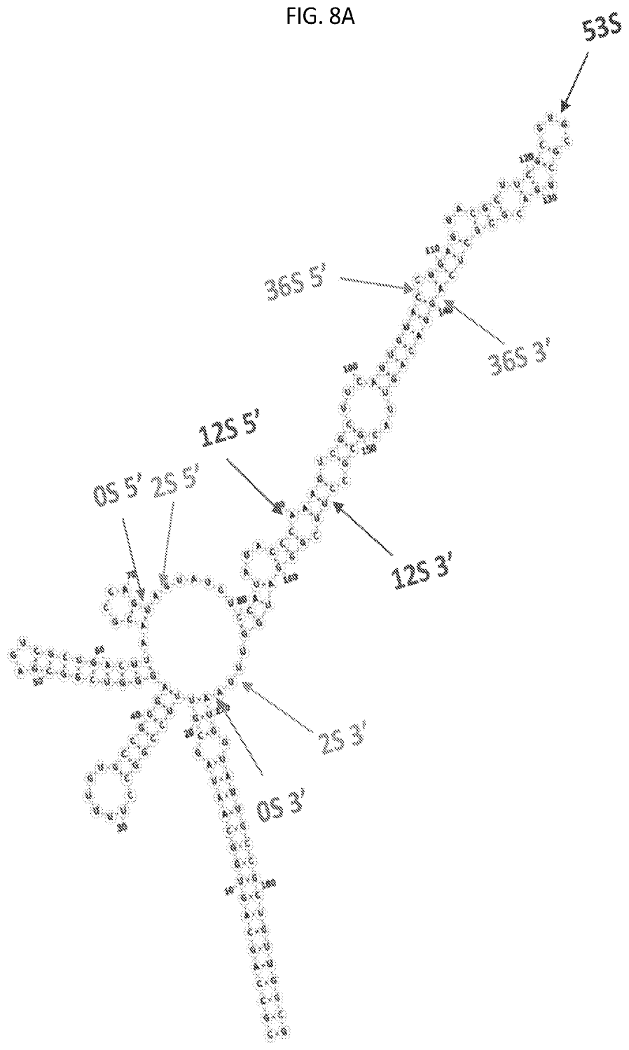

Example 4: A retron transcript comprising a structural motif at the terminus of the retron-gRNA transcript increases HDR (Homology directed repair) rates. A plasmid expressing a fusion RNA of retron (msrimsd) RNA with CRISPR gRNA with another plasmid expressing Cas9-RT fusion was co-transfected into HEK293 cells where the gRNA was positioned either upstream or downstream of the retron msr/msd, but where the fusion transcript was also made comprising a structural motif on the 3′ end of the transcript (FIG. 7A). Structural motifs 5′ to the retron transcript are also expected to provide the same benefit. The structural motifs were found to significantly increase HDR rates, which is illustrated in FIG. 7B.

Example 5: To determine whether shorter msr/msd sequences flanking the heterologous targeting sequence can affect editing efficiency, several constructs were made in which the heterologous sequence targeting the HEK3 locus in HEK293T cells was inserted into full size and truncated versions of the Ec107 and Mx162 retron msr/msd region, as indicated in FIGS. 8A and 9A. HDR editing efficiency, as shown in FIGS. 8B and 9B surprisingly demonstrates that sequential deletion of the secondary stem-loop structure leads to greater HDR frequency (see Ec107 2S, Ec107 12S and Mx162 7S). However, the results suggest that once the base of the stem-loop is disrupted, as in Ec107 0S and Mx162 OS, HDR efficiency is no longer optimized via truncation. Although, the efficiency may not be increased in the OS constructs, HDR is still accomplished and can be useful.

Example 6: To determine if the size of the homology arms and the sense or reverse complement orientation in the targeting sequence impact HDR efficiency in retron-mediated editing, HEK293T cells were transiently transfected with 1) plasmid expressing Cas9 fused to a retron RT (Ec86) and 2) a plasmid expressing an Ec86 retron guide RNA targeting the HEK3 locus with varying lengths and strands (RC=reverse complement; S=sense). Genomic DNA was harvested three days post-transfection and analyzed by next generation sequencing. The results in FIG. 6 show that longer homology arms in the reverse complement orientation leads to higher HDR frequency, as indicated for Ec86 5′ (100/100) RC and Ec86 5′ (200/200) RC.

Example 7: To examine the role that the number and position of nuclear location signals (NLSs) play in retron-mediated HDR, several fusion protein expression cassettes were made as shown in FIG. 11A. These fusion proteins contained the Cas9 protein fused to the Ec107 RT via a XTEN peptide linker. The types and number of NLSs in the constructs are described in FIG. 11A, and the resulting HDR efficiency for the constructs are shown in FIG. 11B.

A pairwise comparison of the HDR efficiency for various constructs indicates that indeed adding more NLS into the base Cas-RT fusion protein can increase HDR efficiency. See, for example, the greater efficiency for 3 NLS 5′ compared to 2 NLS. However, the position and type of NLS signal being added can determine whether HDR efficiency is increased, relatively unaffected, or decreased. When an additional SV40 NLS was added to 3 NLS 5′, resulting in 4 NLS, the HDR efficiency was decreased. Similarly, when an additional SV40 NLS was inserted into the XTEN linker in 2 NLS, resulting in 3 NLS Linker, the HDR efficiency decreased. Thus, while in general more NLS elements can increase the editing efficiency of Cas-RT fusion proteins, the type of the added NLS and the position of the NLS within the fusion protein influences or abolishes that increase.

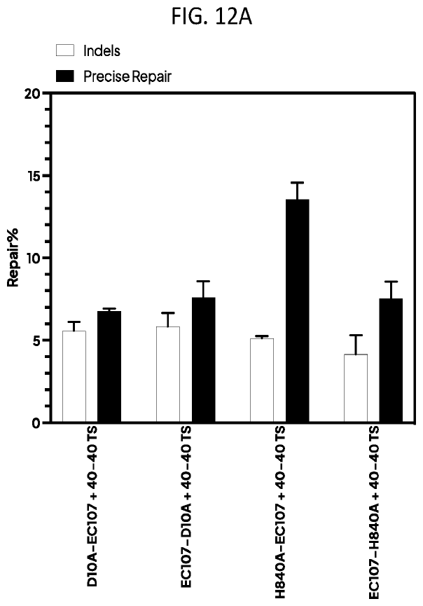

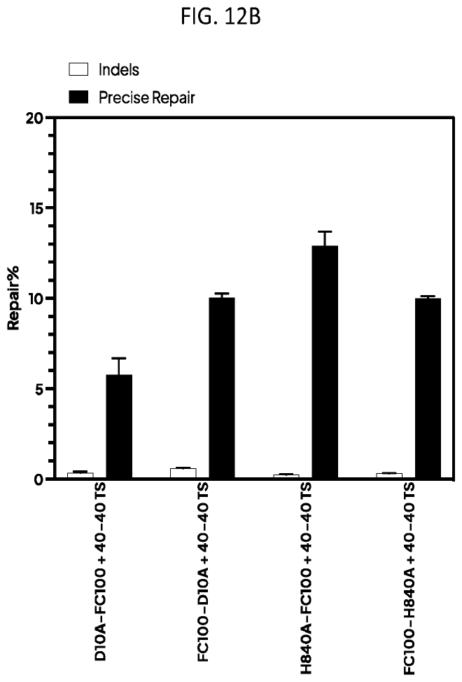

Example 8: To assess whether the orientation of nCas9 and retron RT fusion affects editing rates, H840A and D10A nickase fusions with either N-terminal RT (RT-nCas9) or C-terminal RT (nCas9-RT) placement were tested with a 40-40 TS repair template. As demonstrated in FIGS. 12A and 12B, both EC107 and FC100 retrons showed highest repair rates with the RT-D10A and H840A-RT conformations when compared to the opposite conformation with each respective nickase. Thus, orientation of RT and nCas9 fusion impacts editing efficiency in a retron independent manner, and in such a way that D10A and H840A may prefer RT fusion opposite to the other nickase.

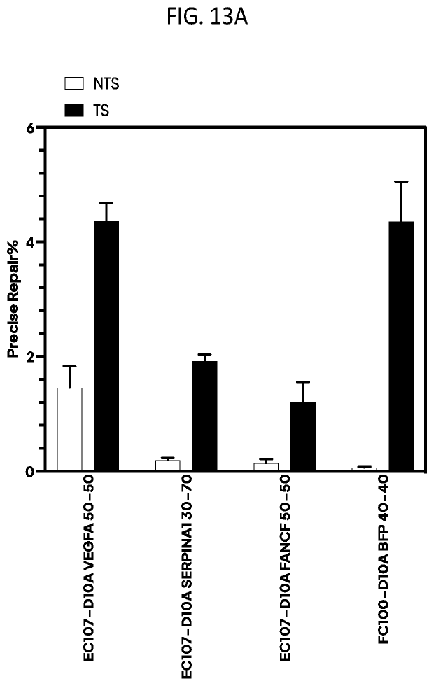

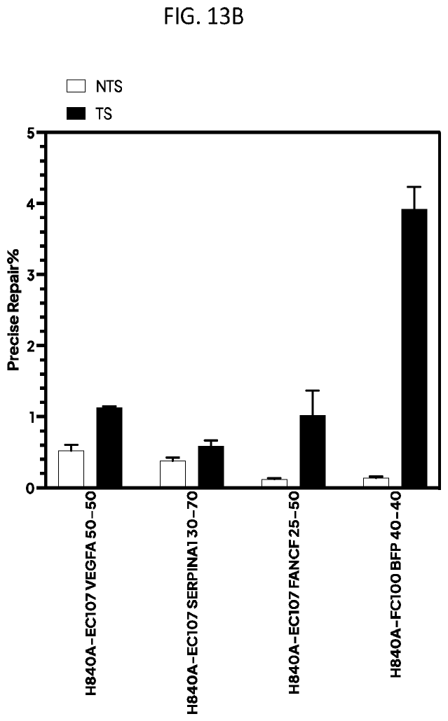

Example 9: To assess the preference of target strand (TS) compared to non-target strand (NTS) repair templates used to repair nickase-generated single stranded breaks, both D10A-RT and H840A-RT editors were paired with a range of templates varying in homology length and strand at multiple loci. The TS repair template outperformed the NTS repair template at all loci, and with both D10A, as shown in FIG. 13A, and H840A editors, as shown in FIG. 13B. This outcome is independent of the optimal template homology and retron at all loci. Considering D10A and H840A cleave opposite DNA strands (TS and NTS, respectively), the regular preference of the TS repair template suggests a unique repair mechanism occurring with each nickase, such that the TS repair template anneals to the nicked strand of DNA when targeted with H840A, and the TS repair template anneals to the intact strand of DNA when targeted with D10A.

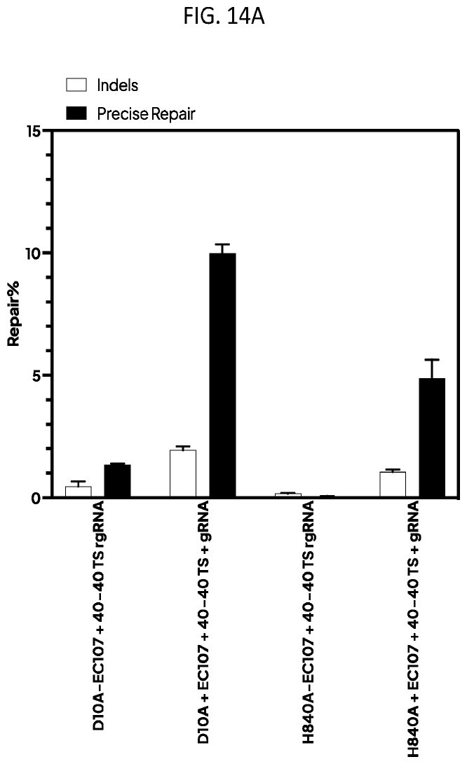

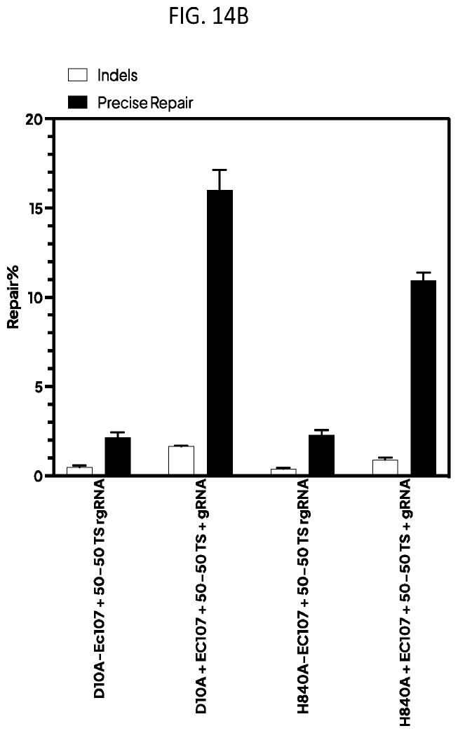

Example 10: To assess the effect of fusing nCas9-RT and ncRNA-gRNA on repair of nickase-generated single strand breaks, a completely unfused system (comprised of nCas9, retron RT, retron ncRNA, and gRNA expressed from 4 plasmids) was compared to a fused system (comprised of nCas9-RT fusion and gRNA-ncRNA fusion expressed from 2 plasmids). The separation of any given component is synergistic, such that the 4-component unfused system has the best editing rates with both D10A and H840A when tested at multiple loci, as shown in FIGS. 14A and 14B. Without wishing to be bound by a particular theory, this effect is likely driven by the increase in cutting efficiency and accessibility of all molecules, which seems to be a more prominent inhibitor to template-based repair with nickase-driven single stranded breaks than to wildtype Cas9-driven double stranded breaks.

The present examples demonstrate the unexpected results that HDR rates can be significantly increased by the embodiments provided for herein, such as by mutating the msd locus.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed is:

1. A method of modifying, or inducing one or more sequence modifications in one or more target nucleic acids of interest at one or more target loci within a genome of a host cell, such as a mammalian cell, the method comprising:
(a) transforming the host cell with one or more vectors encoding a heterologous nucleic acid molecule and a guide RNA (gRNA), wherein the heterologous nucleic acid molecule comprises a first inverted repeat nucleic acid molecule sequence upstream of a coding region and a second inverted repeat nucleic acid molecule sequence downstream of the coding region, wherein the coding region comprises a nucleic acid molecule comprising:
(i) an msr locus and an msd locus comprising the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2; and
(ii) a donor DNA sequence within the msd locus,
wherein the msr locus and the msd locus form a RT binding region; and
(b) culturing the host cell or transformed progeny of the host cell under conditions sufficient for expressing from the one or more vectors the heterologous nucleic acid molecule comprising a retron transcript and a gRNA molecule to induce one or more sequence modifications in one or more target nucleic acids of interest at the one or more target loci within the genome.

2. The method of claim 1, wherein the coding region comprises a nucleic acid molecule comprising:
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 7 (CGCCAGTA), 4, 5, 6, 8, 14, 15, 16, 17, or 18, upstream of the donor DNA sequence; and
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 12 (TAATGG), 9, 10, 11, 13, 19, 20, 21, 22, or 23, downstream of the donor DNA sequence.

3. The method of claim 2, wherein the coding region comprises a nucleic acid molecule comprising:
a nucleic acid sequence of SEQ ID NO: 7 (CGCCAGTA) upstream of the donor DNA sequence; and a nucleic acid sequence of SEQ ID NO: 12 (TAATGG) downstream of the donor DNA sequence;
a nucleic acid sequence of SEQ ID NO: 4 upstream of the donor DNA sequence; and a nucleic acid sequence of SEQ ID NO: 9 downstream of the donor DNA sequence;
a nucleic acid sequence of SEQ ID NO: 4 upstream of the donor DNA sequence; and a nucleic acid sequence of SEQ ID NO: 10 downstream of the donor DNA sequence;
a nucleic acid sequence of SEQ ID NO: 4 upstream of the donor DNA sequence; and a nucleic acid sequence of SEQ ID NO: 11 downstream of the donor DNA sequence;
a nucleic acid sequence of SEQ ID NO: 4 upstream of the donor DNA sequence; and a nucleic acid sequence of SEQ ID NO: 12 downstream of the donor DNA sequence;
a nucleic acid sequence of SEQ ID NO: 4 upstream of the donor DNA sequence; and a nucleic acid sequence of SEQ ID NO: 13 downstream of the donor DNA sequence;
a nucleic acid sequence of SEQ ID NO: 5 upstream of the donor DNA sequence; and a nucleic acid sequence of SEQ ID NO: 9 downstream of the donor DNA sequence;
a nucleic acid sequence of SEQ ID NO: 5 upstream of the donor DNA sequence; and a nucleic acid sequence of SEQ ID NO: 10 downstream of the donor DNA sequence;
a nucleic acid sequence of SEQ ID NO: 5 upstream of the donor DNA sequence; and a nucleic acid sequence of SEQ ID NO: 11 downstream of the donor DNA sequence;
a nucleic acid sequence of SEQ ID NO: 5 upstream of the donor DNA sequence; and a nucleic acid sequence of SEQ ID NO: 12 downstream of the donor DNA sequence;
a nucleic acid sequence of SEQ ID NO: 5 upstream of the donor DNA sequence; and a nucleic acid sequence of SEQ ID NO: 13 downstream of the donor DNA sequence;
a nucleic acid sequence of SEQ ID NO: 6 upstream of the donor DNA sequence; and a nucleic acid sequence of SEQ ID NO: 9 downstream of the donor DNA sequence;
a nucleic acid sequence of SEQ ID NO: 6 upstream of the donor DNA sequence; and a nucleic acid sequence of SEQ ID NO: 10 downstream of the donor DNA sequence;
a nucleic acid sequence of SEQ ID NO: 6 upstream of the donor DNA sequence; and a nucleic acid sequence of SEQ ID NO: 11 downstream of the donor DNA sequence;
a nucleic acid sequence of SEQ ID NO: 6 upstream of the donor DNA sequence; and a nucleic acid sequence of SEQ ID NO: 12 downstream of the donor DNA sequence;
a nucleic acid sequence of SEQ ID NO: 6 upstream of the donor DNA sequence; and a nucleic acid sequence of SEQ ID NO: 13 downstream of the donor DNA sequence;
a nucleic acid sequence of SEQ ID NO: 7 upstream of the donor DNA sequence; and a nucleic acid sequence of SEQ ID NO: 9 downstream of the donor DNA sequence;
a nucleic acid sequence of SEQ ID NO: 7 upstream of the donor DNA sequence; and a nucleic acid sequence of SEQ ID NO: 10 downstream of the donor DNA sequence;
a nucleic acid sequence of SEQ ID NO: 7 upstream of the donor DNA sequence; and a nucleic acid sequence of SEQ ID NO: 11 downstream of the donor DNA sequence;
a nucleic acid sequence of SEQ ID NO: 7 upstream of the donor DNA sequence; and a nucleic acid sequence of SEQ ID NO: 13 downstream of the donor DNA sequence;
a nucleic acid sequence of SEQ ID NO: 8 upstream of the donor DNA sequence; and a nucleic acid sequence of SEQ ID NO: 9 downstream of the donor DNA sequence;
a nucleic acid sequence of SEQ ID NO: 8 upstream of the donor DNA sequence; and a nucleic acid sequence of SEQ ID NO: 10 downstream of the donor DNA sequence;
a nucleic acid sequence of SEQ ID NO: 8 upstream of the donor DNA sequence; and a nucleic acid sequence of SEQ ID NO: 11 downstream of the donor DNA sequence;
a nucleic acid sequence of SEQ ID NO: 8 upstream of the donor DNA sequence; and a nucleic acid sequence of SEQ ID NO: 12 downstream of the donor DNA sequence; or
a nucleic acid sequence of SEQ ID NO: 8 upstream of the donor DNA sequence; and a nucleic acid sequence of SEQ ID NO: 13 downstream of the donor DNA sequence.

4. The method of claim 1, wherein the msd locus comprises a deletion of at least 1 nucleotide as compared to SEQ ID NO: 1, or 2.

5. The method of claim 4, wherein the msd locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to SEQ ID NO: 1, or 2.

6. The method of claim 2, wherein the msd locus upstream (to the 5′ of) of the donor DNA sequence comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18.

7. The method of claim 4, wherein the msd locus upstream (to the 5′ of) of the donor DNA sequence comprises a nucleic acid sequence of any one of SEQ ID NO: 4, 5, 6, 7, 8, 14, 15, 16, 17, or 18.

8. The method of claim 2, wherein the msd locus downstream (to the 3′ of) of the donor DNA sequence comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23.

9. The method of claim 8, wherein the msd locus downstream (to the 3′ of) of the donor DNA sequence comprises a nucleic acid sequence of any one of SEQ ID NO: 9, 10, 11, 12, 13, 19, 20, 21, 22, or 23.

10. The method of claim 2, wherein the coding region comprises a nucleic acid molecule comprising:
a nucleic acid sequence of any one of SEQ ID NO: 4, 5, 6, 7, 8, 14, 15, 16, 17, or 18, upstream of the donor DNA sequence; and
a nucleic acid sequence of any one of SEQ ID NO: 9, 10, 11, 12, 13, 19, 20, 21, 22, or 23, downstream of the donor DNA sequence.

11. The method of claim 1, wherein the coding region comprises a nucleic acid molecule having a formula of 5′-M1-X1-M2-3′, wherein M1 is a fragment of the msr locus and the msd locus, X1 is the donor DNA sequence coding region; and M2 is a fragment of the msr locus and the msd locus.

12. The method of claim 11, wherein the nucleic acid molecule comprises:
an M1 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and
an M2 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.

13. The method of claim 12, wherein the nucleic acid molecule comprises
an M1 having a nucleic acid sequence of SEQ ID NO: 24; and an M2 having a nucleic acid sequence of SEQ ID NO: 34;
an M1 having a nucleic acid sequence of SEQ ID NO: 24; and an M2 having a nucleic acid sequence of SEQ ID NO: 35;
an M1 having a nucleic acid sequence of SEQ ID NO: 24; and an M2 having a nucleic acid sequence of SEQ ID NO: 36;
an M1 having a nucleic acid sequence of SEQ ID NO: 24; and an M2 having a nucleic acid sequence of SEQ ID NO: 37;
an M1 having a nucleic acid sequence of SEQ ID NO: 24; and an M2 having a nucleic acid sequence of SEQ ID NO: 38;
an M1 having a nucleic acid sequence of SEQ ID NO: 24; and an M2 having a nucleic acid sequence of SEQ ID NO: 39;
an M1 having a nucleic acid sequence of SEQ ID NO: 24; and an M2 having a nucleic acid sequence of SEQ ID NO: 40;
an M1 having a nucleic acid sequence of SEQ ID NO: 24; and an M2 having a nucleic acid sequence of SEQ ID NO: 41;
an M1 having a nucleic acid sequence of SEQ ID NO: 24; and an M2 having a nucleic acid sequence of SEQ ID NO: 42;
an M1 having a nucleic acid sequence of SEQ ID NO: 24; and an M2 having a nucleic acid sequence of SEQ ID NO: 43;
an M1 having a nucleic acid sequence of SEQ ID NO: 25; and an M2 having a nucleic acid sequence of SEQ ID NO: 34;
an M1 having a nucleic acid sequence of SEQ ID NO: 25; and an M2 having a nucleic acid sequence of SEQ ID NO: 35;
an M1 having a nucleic acid sequence of SEQ ID NO: 25; and an M2 having a nucleic acid sequence of SEQ ID NO: 36;
an M1 having a nucleic acid sequence of SEQ ID NO: 25; and an M2 having a nucleic acid sequence of SEQ ID NO: 37;
an M1 having a nucleic acid sequence of SEQ ID NO: 25; and an M2 having a nucleic acid sequence of SEQ ID NO: 38;
an M1 having a nucleic acid sequence of SEQ ID NO: 25; and an M2 having a nucleic acid sequence of SEQ ID NO: 39;
an M1 having a nucleic acid sequence of SEQ ID NO: 25; and an M2 having a nucleic acid sequence of SEQ ID NO: 40;
an M1 having a nucleic acid sequence of SEQ ID NO: 25; and an M2 having a nucleic acid sequence of SEQ ID NO: 41;
an M1 having a nucleic acid sequence of SEQ ID NO: 25; and an M2 having a nucleic acid sequence of SEQ ID NO: 42;
an M1 having a nucleic acid sequence of SEQ ID NO: 25; and an M2 having a nucleic acid sequence of SEQ ID NO: 43;
an M1 having a nucleic acid sequence of SEQ ID NO: 26; and an M2 having a nucleic acid sequence of SEQ ID NO: 34;
an M1 having a nucleic acid sequence of SEQ ID NO: 26; and an M2 having a nucleic acid sequence of SEQ ID NO: 35;
an M1 having a nucleic acid sequence of SEQ ID NO: 26; and an M2 having a nucleic acid sequence of SEQ ID NO: 36;
an M1 having a nucleic acid sequence of SEQ ID NO: 26; and an M2 having a nucleic acid sequence of SEQ ID NO: 37;
an M1 having a nucleic acid sequence of SEQ ID NO: 26; and an M2 having a nucleic acid sequence of SEQ ID NO: 38;
an M1 having a nucleic acid sequence of SEQ ID NO: 26; and an M2 having a nucleic acid sequence of SEQ ID NO: 39;
an M1 having a nucleic acid sequence of SEQ ID NO: 26; and an M2 having a nucleic acid sequence of SEQ ID NO: 40;
an M1 having a nucleic acid sequence of SEQ ID NO: 26; and an M2 having a nucleic acid sequence of SEQ ID NO: 41;
an M1 having a nucleic acid sequence of SEQ ID NO: 26; and an M2 having a nucleic acid sequence of SEQ ID NO: 42;
an M1 having a nucleic acid sequence of SEQ ID NO: 26; and an M2 having a nucleic acid sequence of SEQ ID NO: 43;
an M1 having a nucleic acid sequence of SEQ ID NO: 27; and an M2 having a nucleic acid sequence of SEQ ID NO: 34;
an M1 having a nucleic acid sequence of SEQ ID NO: 27; and an M2 having a nucleic acid sequence of SEQ ID NO: 35;
an M1 having a nucleic acid sequence of SEQ ID NO: 27; and an M2 having a nucleic acid sequence of SEQ ID NO: 36;
an M1 having a nucleic acid sequence of SEQ ID NO: 27; and an M2 having a nucleic acid sequence of SEQ ID NO: 37;
an M1 having a nucleic acid sequence of SEQ ID NO: 27; and an M2 having a nucleic acid sequence of SEQ ID NO: 38;
an M1 having a nucleic acid sequence of SEQ ID NO: 27; and an M2 having a nucleic acid sequence of SEQ ID NO: 39;
an M1 having a nucleic acid sequence of SEQ ID NO: 27; and an M2 having a nucleic acid sequence of SEQ ID NO: 40;
an M1 having a nucleic acid sequence of SEQ ID NO: 27; and an M2 having a nucleic acid sequence of SEQ ID NO: 41;
an M1 having a nucleic acid sequence of SEQ ID NO: 27; and an M2 having a nucleic acid sequence of SEQ ID NO: 42;
an M1 having a nucleic acid sequence of SEQ ID NO: 27; and an M2 having a nucleic acid sequence of SEQ ID NO: 43;
an M1 having a nucleic acid sequence of SEQ ID NO: 28; and an M2 having a nucleic acid sequence of SEQ ID NO: 34;
an M1 having a nucleic acid sequence of SEQ ID NO: 28; and an M2 having a nucleic acid sequence of SEQ ID NO: 35;
an M1 having a nucleic acid sequence of SEQ ID NO: 28; and an M2 having a nucleic acid sequence of SEQ ID NO: 36;
an M1 having a nucleic acid sequence of SEQ ID NO: 28; and an M2 having a nucleic acid sequence of SEQ ID NO: 37;
an M1 having a nucleic acid sequence of SEQ ID NO: 28; and an M2 having a nucleic acid sequence of SEQ ID NO: 38;
an M1 having a nucleic acid sequence of SEQ ID NO: 28; and an M2 having a nucleic acid sequence of SEQ ID NO: 39;
an M1 having a nucleic acid sequence of SEQ ID NO: 28; and an M2 having a nucleic acid sequence of SEQ ID NO: 40;
an M1 having a nucleic acid sequence of SEQ ID NO: 28; and an M2 having a nucleic acid sequence of SEQ ID NO: 41;
an M1 having a nucleic acid sequence of SEQ ID NO: 28; and an M2 having a nucleic acid sequence of SEQ ID NO: 42;
an M1 having a nucleic acid sequence of SEQ ID NO: 28; and an M2 having a nucleic acid sequence of SEQ ID NO: 43;
an M1 having a nucleic acid sequence of SEQ ID NO: 29; and an M2 having a nucleic acid sequence of SEQ ID NO: 34;
an M1 having a nucleic acid sequence of SEQ ID NO: 29; and an M2 having a nucleic acid sequence of SEQ ID NO: 35;
an M1 having a nucleic acid sequence of SEQ ID NO: 29; and an M2 having a nucleic acid sequence of SEQ ID NO: 36;
an M1 having a nucleic acid sequence of SEQ ID NO: 29; and an M2 having a nucleic acid sequence of SEQ ID NO: 37;
an M1 having a nucleic acid sequence of SEQ ID NO: 29; and an M2 having a nucleic acid sequence of SEQ ID NO: 38;
an M1 having a nucleic acid sequence of SEQ ID NO: 29; and an M2 having a nucleic acid sequence of SEQ ID NO: 39;
an M1 having a nucleic acid sequence of SEQ ID NO: 29; and an M2 having a nucleic acid sequence of SEQ ID NO: 40;
an M1 having a nucleic acid sequence of SEQ ID NO: 29; and an M2 having a nucleic acid sequence of SEQ ID NO: 41;
an M1 having a nucleic acid sequence of SEQ ID NO: 29; and an M2 having a nucleic acid sequence of SEQ ID NO: 42;
an M1 having a nucleic acid sequence of SEQ ID NO: 29; and an M2 having a nucleic acid sequence of SEQ ID NO: 43;
an M1 having a nucleic acid sequence of SEQ ID NO: 30; and an M2 having a nucleic acid sequence of SEQ ID NO: 34;
an M1 having a nucleic acid sequence of SEQ ID NO: 30; and an M2 having a nucleic acid sequence of SEQ ID NO: 35;
an M1 having a nucleic acid sequence of SEQ ID NO: 30; and an M2 having a nucleic acid sequence of SEQ ID NO: 36;
an M1 having a nucleic acid sequence of SEQ ID NO: 30; and an M2 having a nucleic acid sequence of SEQ ID NO: 37;
an M1 having a nucleic acid sequence of SEQ ID NO: 30; and an M2 having a nucleic acid sequence of SEQ ID NO: 38;
an M1 having a nucleic acid sequence of SEQ ID NO: 30; and an M2 having a nucleic acid sequence of SEQ ID NO: 39;
an M1 having a nucleic acid sequence of SEQ ID NO: 30; and an M2 having a nucleic acid sequence of SEQ ID NO: 40;
an M1 having a nucleic acid sequence of SEQ ID NO: 30; and an M2 having a nucleic acid sequence of SEQ ID NO: 41;
an M1 having a nucleic acid sequence of SEQ ID NO: 30; and an M2 having a nucleic acid sequence of SEQ ID NO: 42;
an M1 having a nucleic acid sequence of SEQ ID NO: 30; and an M2 having a nucleic acid sequence of SEQ ID NO: 43;
an M1 having a nucleic acid sequence of SEQ ID NO: 31; and an M2 having a nucleic acid sequence of SEQ ID NO: 34;
an M1 having a nucleic acid sequence of SEQ ID NO: 31; and an M2 having a nucleic acid sequence of SEQ ID NO: 35;
an M1 having a nucleic acid sequence of SEQ ID NO: 31; and an M2 having a nucleic acid sequence of SEQ ID NO: 36;
an M1 having a nucleic acid sequence of SEQ ID NO: 31; and an M2 having a nucleic acid sequence of SEQ ID NO: 37;
an M1 having a nucleic acid sequence of SEQ ID NO: 31; and an M2 having a nucleic acid sequence of SEQ ID NO: 38;
an M1 having a nucleic acid sequence of SEQ ID NO: 31; and an M2 having a nucleic acid sequence of SEQ ID NO: 39;
an M1 having a nucleic acid sequence of SEQ ID NO: 31; and an M2 having a nucleic acid sequence of SEQ ID NO: 40;
an M1 having a nucleic acid sequence of SEQ ID NO: 31; and an M2 having a nucleic acid sequence of SEQ ID NO: 41;
an M1 having a nucleic acid sequence of SEQ ID NO: 31; and an M2 having a nucleic acid sequence of SEQ ID NO: 42;
an M1 having a nucleic acid sequence of SEQ ID NO: 31; and an M2 having a nucleic acid sequence of SEQ ID NO: 43;
an M1 having a nucleic acid sequence of SEQ ID NO: 32; and an M2 having a nucleic acid sequence of SEQ ID NO: 34;
an M1 having a nucleic acid sequence of SEQ ID NO: 32; and an M2 having a nucleic acid sequence of SEQ ID NO: 35;
an M1 having a nucleic acid sequence of SEQ ID NO: 32; and an M2 having a nucleic acid sequence of SEQ ID NO: 36;
an M1 having a nucleic acid sequence of SEQ ID NO: 32; and an M2 having a nucleic acid sequence of SEQ ID NO: 37;
an M1 having a nucleic acid sequence of SEQ ID NO: 32; and an M2 having a nucleic acid sequence of SEQ ID NO: 38;
an M1 having a nucleic acid sequence of SEQ ID NO: 32; and an M2 having a nucleic acid sequence of SEQ ID NO: 39;
an M1 having a nucleic acid sequence of SEQ ID NO: 32; and an M2 having a nucleic acid sequence of SEQ ID NO: 40;
an M1 having a nucleic acid sequence of SEQ ID NO: 32; and an M2 having a nucleic acid sequence of SEQ ID NO: 41;
an M1 having a nucleic acid sequence of SEQ ID NO: 32; and an M2 having a nucleic acid sequence of SEQ ID NO: 42;
an M1 having a nucleic acid sequence of SEQ ID NO: 32; and an M2 having a nucleic acid sequence of SEQ ID NO: 43;
an M1 having a nucleic acid sequence of SEQ ID NO: or 33; and an M2 having a nucleic acid sequence of SEQ ID NO: 34;
an M1 having a nucleic acid sequence of SEQ ID NO: or 33; and an M2 having a nucleic acid sequence of SEQ ID NO: 35;
an M1 having a nucleic acid sequence of SEQ ID NO: or 33; and an M2 having a nucleic acid sequence of SEQ ID NO: 36;
an M1 having a nucleic acid sequence of SEQ ID NO: or 33; and an M2 having a nucleic acid sequence of SEQ ID NO: 37;
an M1 having a nucleic acid sequence of SEQ ID NO: or 33; and an M2 having a nucleic acid sequence of SEQ ID NO: 38;
an M1 having a nucleic acid sequence of SEQ ID NO: or 33; and an M2 having a nucleic acid sequence of SEQ ID NO: 39;
an M1 having a nucleic acid sequence of SEQ ID NO: or 33; and an M2 having a nucleic acid sequence of SEQ ID NO: 40;
an M1 having a nucleic acid sequence of SEQ ID NO: or 33; and an M2 having a nucleic acid sequence of SEQ ID NO: 41;
an M1 having a nucleic acid sequence of SEQ ID NO: or 33; and an M2 having a nucleic acid sequence of SEQ ID NO: 42; or
an M1 having a nucleic acid sequence of SEQ ID NO: or 33; and an M2 having a nucleic acid sequence of SEQ ID NO: 43.

14. The method of any one of claims 1-13, wherein the donor DNA sequence is a target strand donor DNA sequence.

15. The method of any one of claims 1-14, wherein the host cell expresses a nuclease and a RT protein.

16. The method claim 15, wherein the nuclease and RT are:
not expressed as a fusion molecule; or
expressed as a fusion protein.

17. The method of claims 15-16, wherein the nuclease is linked to the RT with a linker.

18. The method of claim 17, wherein the linker is a peptide linker.

19. The method of any one of claims 15-18, wherein the C-terminus of the nuclease is linked to the N-terminus of the RT, or the N-terminus of the nuclease is linked to the C-terminus of the RT.

20. The method of any one of claims 1-19, wherein the host cell expresses a RT and a single-stranded annealing protein (SSAP).

21. The method of any one of claims 15-20, wherein the nuclease is a Cas nuclease, such as a catalytically active nuclease, a nickase Cas nuclease, a catalytically inactive Cas nuclease, a Cas9 nuclease, nickase Cas9 nuclease, a catalytically inactive Cas9 nuclease, Cas9D10A nickase, Cas9H840A nickase, or a Cas having D10A and H840A mutations.

22. The method of any one of claims 15-21, wherein the RT is a fusion protein comprising a polypeptide having the formula of: of (N1)q-C1-L1-(N2)qq-R1-(N3)qqq, wherein C1 is a nuclease, L1 is a peptide linker, R1 is a RT protein, and N1 and N2 are each independently, a NLS sequence, and wherein q, qq, or qqq, are each independently, 0, 1, 2, or 3.

23. The method of claim 22, wherein the polypeptide has the formula of: 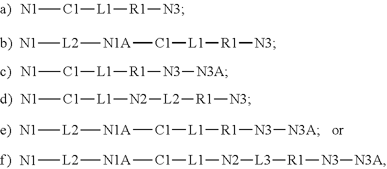
wherein:
each N1, N1A, N2, N3, or N3A is, independently, a NLS sequence;
each L1, L2, or L3 is independently, a linker amino acid sequence; and
wherein each NLS sequence is identical or different.

24. The method of any one of claims 22-23, wherein N1, N1A, N2, N3, or N3A, each independently comprise a SV40 NLS sequence, a cMyc NLS sequence, or a Nuceloplasmin NLS sequence.

25. The method of any one of claims 15-24, wherein R1 comprises a RT sequence Ec86, Ec48, Ec73, Ec107, or Sa163.

26. The method of any one of claims 22-25, wherein the polypeptide comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 70, 71, 72, 73, or 74.

27. The method of claim 26, wherein the polypeptide comprises an amino acid sequence of any one of SEQ ID NO: 70, 71, 72, 73, or 74.

28. The method of any one of claims 1-27, wherein the retron transcript and the gRNA are not linked together; or wherein the retron transcript and the gRNA are linked together, such as part of the same contiguous transcript, or not part of the same contiguous transcript.

29. The method of claim 28, wherein the retron and gRNA are covalently linked together.

30. The method of any one of claims 1-29, wherein the retron transcript and the gRNA comprises a structured motif at a 5′ or a 3′ end of the retron transcript and the gRNA, such as evoPreQ, tevopreQ mpKnot, or exoribonuclease-resistant RNA motif.

31. The method of claim 30, wherein the structured motif comprises a nucleic acid sequence selected from any one of SEQ ID NO: 75, 76, 77, 78, 79, 80, 81, or 82.

32. The method of any one of claims 1-31, wherein the retron transcript linked to the gRNA molecule comprises a formula A1-Rt-L1n-gRNA-A2, wherein:
(a) A1 is the structured motif, or is absent;
Rt is the retron transcript;
L1n is a nucleotide linker from about 5 to about 40, about 8 to about 35, about 9, or about 33 basepairs, or is absent; and
A2 is the structured motif, or is absent;
(b) A1 is the structured motif;
Rt is the retron transcript;
L1n is a nucleotide linker from about 5 to about 40, about 8 to about 35, about 9, or about 33 basepairs; and
A2 is absent; or
(c) A1 is absent;
Rt is the retron transcript;
L1 is a nucleotide linker from about 5 to about 40, about 8 to about 35, about 9, or about 33 base pairs; and
A2 is the structured motif.

33. A method of treating a disease or condition by modifying one or more target nucleic acids of interest at one or more target loci within a genome of a host cell, such as a mammalian cells, the method comprising the method of any one of claims 1-32.

34. A method for modifying one or more target nucleic acids of interest at one or more target loci within a genome of a host cell, such as a mammalian cells, the method comprising the method of any one of claims 1-32.

35. A retron-guide RNA (gRNA) cassette comprising:
a retron comprising:
a first inverted repeat sequence coding region;
an msr locus and an msd locus;
a donor DNA sequence within the msd locus; and
a second inverted repeat sequence coding region; and
optionally a gRNA coding region,
wherein the msr locus and the msd locus comprise the nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 1, or 2.

36. The cassette of claim 35, wherein the retron comprises a nucleic acid molecule comprising:
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 7, 4, 5, 6, 8, 14, 15, 16, 17, or 18, upstream of the donor DNA sequence; and
a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 12, 9, 10, 11, 13, 19, 20, 21, 22, or 23, downstream of the donor DNA sequence.

37. The cassette of claim 36, wherein the retron comprises a nucleic acid molecule comprising:
a nucleic acid sequence of SEQ ID NO: 7 upstream of the donor DNA sequence; and a nucleic acid sequence of SEQ ID NO: 12 downstream of the donor DNA sequence;
a nucleic acid sequence of SEQ ID NO: 4 upstream of the donor DNA sequence; and a nucleic acid sequence of SEQ ID NO: 9 downstream of the donor DNA sequence;
a nucleic acid sequence of SEQ ID NO: 4 upstream of the donor DNA sequence; and a nucleic acid sequence of SEQ ID NO: 10 downstream of the donor DNA sequence;
a nucleic acid sequence of SEQ ID NO: 4 upstream of the donor DNA sequence; and a nucleic acid sequence of SEQ ID NO: 11 downstream of the donor DNA sequence;
a nucleic acid sequence of SEQ ID NO: 4 upstream of the donor DNA sequence; and a nucleic acid sequence of SEQ ID NO: 12 downstream of the donor DNA sequence;
a nucleic acid sequence of SEQ ID NO: 4 upstream of the donor DNA sequence; and a nucleic acid sequence of SEQ ID NO: 13 downstream of the donor DNA sequence;
a nucleic acid sequence of SEQ ID NO: 5 upstream of the donor DNA sequence; and a nucleic acid sequence of SEQ ID NO: 9 downstream of the donor DNA sequence;
a nucleic acid sequence of SEQ ID NO: 5 upstream of the donor DNA sequence; and a nucleic acid sequence of SEQ ID NO: 10 downstream of the donor DNA sequence;
a nucleic acid sequence of SEQ ID NO: 5 upstream of the donor DNA sequence; and a nucleic acid sequence of SEQ ID NO: 11 downstream of the donor DNA sequence;
a nucleic acid sequence of SEQ ID NO: 5 upstream of the donor DNA sequence; and a nucleic acid sequence of SEQ ID NO: 12 downstream of the donor DNA sequence;
a nucleic acid sequence of SEQ ID NO: 5 upstream of the donor DNA sequence; and a nucleic acid sequence of SEQ ID NO: 13 downstream of the donor DNA sequence;
a nucleic acid sequence of SEQ ID NO: 6 upstream of the donor DNA sequence; and a nucleic acid sequence of SEQ ID NO: 9 downstream of the donor DNA sequence;
a nucleic acid sequence of SEQ ID NO: 6 upstream of the donor DNA sequence; and a nucleic acid sequence of SEQ ID NO: 10 downstream of the donor DNA sequence;
a nucleic acid sequence of SEQ ID NO: 6 upstream of the donor DNA sequence; and a nucleic acid sequence of SEQ ID NO: 11 downstream of the donor DNA sequence;
a nucleic acid sequence of SEQ ID NO: 6 upstream of the donor DNA sequence; and a nucleic acid sequence of SEQ ID NO: 12 downstream of the donor DNA sequence;
a nucleic acid sequence of SEQ ID NO: 6 upstream of the donor DNA sequence; and a nucleic acid sequence of SEQ ID NO: 13 downstream of the donor DNA sequence;
a nucleic acid sequence of SEQ ID NO: 7 upstream of the donor DNA sequence; and a nucleic acid sequence of SEQ ID NO: 9 downstream of the donor DNA sequence;
a nucleic acid sequence of SEQ ID NO: 7 upstream of the donor DNA sequence; and a nucleic acid sequence of SEQ ID NO: 10 downstream of the donor DNA sequence;
a nucleic acid sequence of SEQ ID NO: 7 upstream of the donor DNA sequence; and a nucleic acid sequence of SEQ ID NO: 11 downstream of the donor DNA sequence;
a nucleic acid sequence of SEQ ID NO: 7 upstream of the donor DNA sequence; and a nucleic acid sequence of SEQ ID NO: 13 downstream of the donor DNA sequence;
a nucleic acid sequence of SEQ ID NO: 8 upstream of the donor DNA sequence; and a nucleic acid sequence of SEQ ID NO: 9 downstream of the donor DNA sequence;
a nucleic acid sequence of SEQ ID NO: 8 upstream of the donor DNA sequence; and a nucleic acid sequence of SEQ ID NO: 10 downstream of the donor DNA sequence;
a nucleic acid sequence of SEQ ID NO: 8 upstream of the donor DNA sequence; and a nucleic acid sequence of SEQ ID NO: 11 downstream of the donor DNA sequence;
a nucleic acid sequence of SEQ ID NO: 8 upstream of the donor DNA sequence; and a nucleic acid sequence of SEQ ID NO: 12 downstream of the donor DNA sequence; or
a nucleic acid sequence of SEQ ID NO: 8 upstream of the donor DNA sequence; and a nucleic acid sequence of SEQ ID NO: 13 downstream of the donor DNA sequence.

38. The cassette of claim 35, wherein the msd locus comprises a deletion of at least 1 nucleotide as compared to SEQ ID NO: 1, or 2.

39. The cassette of claim 38, wherein the msd locus comprises a deletion of about 1 to 150, about 1 to about 125, about 1 to about 100, about 1 to about 90, about 1 to about 80, about 1 to about 70, about 1 to about 60, about 1 to about 50, about 1 to about 40, about 1 to about 30, about 1 to about 20, or about 1 to about 10 nucleotides as compared to SEQ ID NO: 1, or 2.

40. The cassette of claim 36, wherein the msd locus upstream (to the 5′ of) of the donor DNA sequence coding region comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 4, 5, 6, 7, 8, 14, 15, 16, 17, or 18.

41. The cassette of claim 40, wherein the msd locus upstream (to the 5′ of) of the donor DNA sequence coding region comprises a nucleic acid sequence of any one of SEQ ID NO: 4, 5, 6, 7, 8, 14, 15, 16, 17, or 18.

42. The cassette of claim 36, wherein the msd locus downstream (to the 3′ of) of the donor DNA sequence coding region comprises a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 9, 10, 11, 12, 13, 19, 20, 21, 22, or 23.

43. The cassette of claim 42, wherein the msd locus downstream (to the 3′ of) of the donor DNA sequence coding region comprises a nucleic acid sequence of any one of SEQ ID NO: 9, 10, 11, 12, 13, 19, 20, 21, 22, or 23.

44. The cassette of claim 36, wherein the retron comprises a nucleic acid molecule comprising:
a nucleic acid sequence of any one of SEQ ID NO: 4, 5, 6, 7, 8, 14, 15, 16, 17, or 18, upstream of the donor DNA sequence coding region coding region; and
a nucleic acid sequence of any one of SEQ ID NO: 9, 10, 11, 12, 13, 19, 20, 21, 22, or 23, downstream of the donor DNA sequence.

45. The cassette of claim 35, wherein the retron comprises a nucleic acid molecule having a formula of 5′-M1-X1-M2-3′, wherein M1 is a fragment of the msr locus and the msd locus, X1 is the donor DNA sequence; and M2 is a fragment of the msd locus and the msr locus.

46. The cassette of claim 45, wherein the nucleic acid molecule comprises:
an M1 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 24, 25, 26, 27, 28, 29, 30, 31, 32, or 33; and
an M2 having a nucleic acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NO: 34, 35, 36, 37, 38, 39, 40, 41, 42, or 43.

47. The cassette of claim 46, wherein the nucleic acid molecule comprises
an M1 having a nucleic acid sequence of SEQ ID NO: 24; and an M2 having a nucleic acid sequence of SEQ ID NO: 34;
an M1 having a nucleic acid sequence of SEQ ID NO: 24; and an M2 having a nucleic acid sequence of SEQ ID NO: 35;
an M1 having a nucleic acid sequence of SEQ ID NO: 24; and an M2 having a nucleic acid sequence of SEQ ID NO: 36;
an M1 having a nucleic acid sequence of SEQ ID NO: 24; and an M2 having a nucleic acid sequence of SEQ ID NO: 37;
an M1 having a nucleic acid sequence of SEQ ID NO: 24; and an M2 having a nucleic acid sequence of SEQ ID NO: 38;
an M1 having a nucleic acid sequence of SEQ ID NO: 24; and an M2 having a nucleic acid sequence of SEQ ID NO: 39;
an M1 having a nucleic acid sequence of SEQ ID NO: 24; and an M2 having a nucleic acid sequence of SEQ ID NO: 40;
an M1 having a nucleic acid sequence of SEQ ID NO: 24; and an M2 having a nucleic acid sequence of SEQ ID NO: 41;
an M1 having a nucleic acid sequence of SEQ ID NO: 24; and an M2 having a nucleic acid sequence of SEQ ID NO: 42;
an M1 having a nucleic acid sequence of SEQ ID NO: 24; and an M2 having a nucleic acid sequence of SEQ ID NO: 43;
an M1 having a nucleic acid sequence of SEQ ID NO: 25; and an M2 having a nucleic acid sequence of SEQ ID NO: 34;
an M1 having a nucleic acid sequence of SEQ ID NO: 25; and an M2 having a nucleic acid sequence of SEQ ID NO: 35;
an M1 having a nucleic acid sequence of SEQ ID NO: 25; and an M2 having a nucleic acid sequence of SEQ ID NO: 36;
an M1 having a nucleic acid sequence of SEQ ID NO: 25; and an M2 having a nucleic acid sequence of SEQ ID NO: 37;
an M1 having a nucleic acid sequence of SEQ ID NO: 25; and an M2 having a nucleic acid sequence of SEQ ID NO: 38;
an M1 having a nucleic acid sequence of SEQ ID NO: 25; and an M2 having a nucleic acid sequence of SEQ ID NO: 39;
an M1 having a nucleic acid sequence of SEQ ID NO: 25; and an M2 having a nucleic acid sequence of SEQ ID NO: 40;
an M1 having a nucleic acid sequence of SEQ ID NO: 25; and an M2 having a nucleic acid sequence of SEQ ID NO: 41;
an M1 having a nucleic acid sequence of SEQ ID NO: 25; and an M2 having a nucleic acid sequence of SEQ ID NO: 42;
an M1 having a nucleic acid sequence of SEQ ID NO: 25; and an M2 having a nucleic acid sequence of SEQ ID NO: 43;
an M1 having a nucleic acid sequence of SEQ ID NO: 26; and an M2 having a nucleic acid sequence of SEQ ID NO: 34;
an M1 having a nucleic acid sequence of SEQ ID NO: 26; and an M2 having a nucleic acid sequence of SEQ ID NO: 35;
an M1 having a nucleic acid sequence of SEQ ID NO: 26; and an M2 having a nucleic acid sequence of SEQ ID NO: 36;
an M1 having a nucleic acid sequence of SEQ ID NO: 26; and an M2 having a nucleic acid sequence of SEQ ID NO: 37;
an M1 having a nucleic acid sequence of SEQ ID NO: 26; and an M2 having a nucleic acid sequence of SEQ ID NO: 38;
an M1 having a nucleic acid sequence of SEQ ID NO: 26; and an M2 having a nucleic acid sequence of SEQ ID NO: 39;
an M1 having a nucleic acid sequence of SEQ ID NO: 26; and an M2 having a nucleic acid sequence of SEQ ID NO: 40;
an M1 having a nucleic acid sequence of SEQ ID NO: 26; and an M2 having a nucleic acid sequence of SEQ ID NO: 41;
an M1 having a nucleic acid sequence of SEQ ID NO: 26; and an M2 having a nucleic acid sequence of SEQ ID NO: 42;
an M1 having a nucleic acid sequence of SEQ ID NO: 26; and an M2 having a nucleic acid sequence of SEQ ID NO: 43;
an M1 having a nucleic acid sequence of SEQ ID NO: 27; and an M2 having a nucleic acid sequence of SEQ ID NO: 34;
an M1 having a nucleic acid sequence of SEQ ID NO: 27; and an M2 having a nucleic acid sequence of SEQ ID NO: 35;
an M1 having a nucleic acid sequence of SEQ ID NO: 27; and an M2 having a nucleic acid sequence of SEQ ID NO: 36;
an M1 having a nucleic acid sequence of SEQ ID NO: 27; and an M2 having a nucleic acid sequence of SEQ ID NO: 37;
an M1 having a nucleic acid sequence of SEQ ID NO: 27; and an M2 having a nucleic acid sequence of SEQ ID NO: 38;
an M1 having a nucleic acid sequence of SEQ ID NO: 27; and an M2 having a nucleic acid sequence of SEQ ID NO: 39;
an M1 having a nucleic acid sequence of SEQ ID NO: 27; and an M2 having a nucleic acid sequence of SEQ ID NO: 40;
an M1 having a nucleic acid sequence of SEQ ID NO: 27; and an M2 having a nucleic acid sequence of SEQ ID NO: 41;
an M1 having a nucleic acid sequence of SEQ ID NO: 27; and an M2 having a nucleic acid sequence of SEQ ID NO: 42;
an M1 having a nucleic acid sequence of SEQ ID NO: 27; and an M2 having a nucleic acid sequence of SEQ ID NO: 43;
an M1 having a nucleic acid sequence of SEQ ID NO: 28; and an M2 having a nucleic acid sequence of SEQ ID NO: 34;
an M1 having a nucleic acid sequence of SEQ ID NO: 28; and an M2 having a nucleic acid sequence of SEQ ID NO: 35;
an M1 having a nucleic acid sequence of SEQ ID NO: 28; and an M2 having a nucleic acid sequence of SEQ ID NO: 36;
an M1 having a nucleic acid sequence of SEQ ID NO: 28; and an M2 having a nucleic acid sequence of SEQ ID NO: 37;
an M1 having a nucleic acid sequence of SEQ ID NO: 28; and an M2 having a nucleic acid sequence of SEQ ID NO: 38;
an M1 having a nucleic acid sequence of SEQ ID NO: 28; and an M2 having a nucleic acid sequence of SEQ ID NO: 39;
an M1 having a nucleic acid sequence of SEQ ID NO: 28; and an M2 having a nucleic acid sequence of SEQ ID NO: 40;
an M1 having a nucleic acid sequence of SEQ ID NO: 28; and an M2 having a nucleic acid sequence of SEQ ID NO: 41;
an M1 having a nucleic acid sequence of SEQ ID NO: 28; and an M2 having a nucleic acid sequence of SEQ ID NO: 42;
an M1 having a nucleic acid sequence of SEQ ID NO: 28; and an M2 having a nucleic acid sequence of SEQ ID NO: 43;
an M1 having a nucleic acid sequence of SEQ ID NO: 29; and an M2 having a nucleic acid sequence of SEQ ID NO: 34;
an M1 having a nucleic acid sequence of SEQ ID NO: 29; and an M2 having a nucleic acid sequence of SEQ ID NO: 35;
an M1 having a nucleic acid sequence of SEQ ID NO: 29; and an M2 having a nucleic acid sequence of SEQ ID NO: 36;
an M1 having a nucleic acid sequence of SEQ ID NO: 29; and an M2 having a nucleic acid sequence of SEQ ID NO: 37;
an M1 having a nucleic acid sequence of SEQ ID NO: 29; and an M2 having a nucleic acid sequence of SEQ ID NO: 38;
an M1 having a nucleic acid sequence of SEQ ID NO: 29; and an M2 having a nucleic acid sequence of SEQ ID NO: 39;
an M1 having a nucleic acid sequence of SEQ ID NO: 29; and an M2 having a nucleic acid sequence of SEQ ID NO: 40;
an M1 having a nucleic acid sequence of SEQ ID NO: 29; and an M2 having a nucleic acid sequence of SEQ ID NO: 41;
an M1 having a nucleic acid sequence of SEQ ID NO: 29; and an M2 having a nucleic acid sequence of SEQ ID NO: 42;
an M1 having a nucleic acid sequence of SEQ ID NO: 29; and an M2 having a nucleic acid sequence of SEQ ID NO: 43;
an M1 having a nucleic acid sequence of SEQ ID NO: 30; and an M2 having a nucleic acid sequence of SEQ ID NO: 34;
an M1 having a nucleic acid sequence of SEQ ID NO: 30; and an M2 having a nucleic acid sequence of SEQ ID NO: 35;
an M1 having a nucleic acid sequence of SEQ ID NO: 30; and an M2 having a nucleic acid sequence of SEQ ID NO: 36;
an M1 having a nucleic acid sequence of SEQ ID NO: 30; and an M2 having a nucleic acid sequence of SEQ ID NO: 37;
an M1 having a nucleic acid sequence of SEQ ID NO: 30; and an M2 having a nucleic acid sequence of SEQ ID NO: 38;
an M1 having a nucleic acid sequence of SEQ ID NO: 30; and an M2 having a nucleic acid sequence of SEQ ID NO: 39;
an M1 having a nucleic acid sequence of SEQ ID NO: 30; and an M2 having a nucleic acid sequence of SEQ ID NO: 40;
an M1 having a nucleic acid sequence of SEQ ID NO: 30; and an M2 having a nucleic acid sequence of SEQ ID NO: 41;
an M1 having a nucleic acid sequence of SEQ ID NO: 30; and an M2 having a nucleic acid sequence of SEQ ID NO: 42;
an M1 having a nucleic acid sequence of SEQ ID NO: 30; and an M2 having a nucleic acid sequence of SEQ ID NO: 43;
an M1 having a nucleic acid sequence of SEQ ID NO: 31; and an M2 having a nucleic acid sequence of SEQ ID NO: 34;
an M1 having a nucleic acid sequence of SEQ ID NO: 31; and an M2 having a nucleic acid sequence of SEQ ID NO: 35;
an M1 having a nucleic acid sequence of SEQ ID NO: 31; and an M2 having a nucleic acid sequence of SEQ ID NO: 36;
an M1 having a nucleic acid sequence of SEQ ID NO: 31; and an M2 having a nucleic acid sequence of SEQ ID NO: 37;
an M1 having a nucleic acid sequence of SEQ ID NO: 31; and an M2 having a nucleic acid sequence of SEQ ID NO: 38;
an M1 having a nucleic acid sequence of SEQ ID NO: 31; and an M2 having a nucleic acid sequence of SEQ ID NO: 39;
an M1 having a nucleic acid sequence of SEQ ID NO: 31; and an M2 having a nucleic acid sequence of SEQ ID NO: 40;
an M1 having a nucleic acid sequence of SEQ ID NO: 31; and an M2 having a nucleic acid sequence of SEQ ID NO: 41;
an M1 having a nucleic acid sequence of SEQ ID NO: 31; and an M2 having a nucleic acid sequence of SEQ ID NO: 42;
an M1 having a nucleic acid sequence of SEQ ID NO: 31; and an M2 having a nucleic acid sequence of SEQ ID NO: 43;
an M1 having a nucleic acid sequence of SEQ ID NO: 32; and an M2 having a nucleic acid sequence of SEQ ID NO: 34;
an M1 having a nucleic acid sequence of SEQ ID NO: 32; and an M2 having a nucleic acid sequence of SEQ ID NO: 35;
an M1 having a nucleic acid sequence of SEQ ID NO: 32; and an M2 having a nucleic acid sequence of SEQ ID NO: 36;
an M1 having a nucleic acid sequence of SEQ ID NO: 32; and an M2 having a nucleic acid sequence of SEQ ID NO: 37;
an M1 having a nucleic acid sequence of SEQ ID NO: 32; and an M2 having a nucleic acid sequence of SEQ ID NO: 38;
an M1 having a nucleic acid sequence of SEQ ID NO: 32; and an M2 having a nucleic acid sequence of SEQ ID NO: 39;
an M1 having a nucleic acid sequence of SEQ ID NO: 32; and an M2 having a nucleic acid sequence of SEQ ID NO: 40;
an M1 having a nucleic acid sequence of SEQ ID NO: 32; and an M2 having a nucleic acid sequence of SEQ ID NO: 41;
an M1 having a nucleic acid sequence of SEQ ID NO: 32; and an M2 having a nucleic acid sequence of SEQ ID NO: 42;
an M1 having a nucleic acid sequence of SEQ ID NO: 32; and an M2 having a nucleic acid sequence of SEQ ID NO: 43;
an M1 having a nucleic acid sequence of SEQ ID NO: 33; and an M2 having a nucleic acid sequence of SEQ ID NO: 34;
an M1 having a nucleic acid sequence of SEQ ID NO: 33; and an M2 having a nucleic acid sequence of SEQ ID NO: 35;
an M1 having a nucleic acid sequence of SEQ ID NO: 33; and an M2 having a nucleic acid sequence of SEQ ID NO: 36;
an M1 having a nucleic acid sequence of SEQ ID NO: 33; and an M2 having a nucleic acid sequence of SEQ ID NO: 37;
an M1 having a nucleic acid sequence of SEQ ID NO: 33; and an M2 having a nucleic acid sequence of SEQ ID NO: 38;
an M1 having a nucleic acid sequence of SEQ ID NO: 33; and an M2 having a nucleic acid sequence of SEQ ID NO: 39;
an M1 having a nucleic acid sequence of SEQ ID NO: 33; and an M2 having a nucleic acid sequence of SEQ ID NO: 40;
an M1 having a nucleic acid sequence of SEQ ID NO: 33; and an M2 having a nucleic acid sequence of SEQ ID NO: 41;
an M1 having a nucleic acid sequence of SEQ ID NO: 33; and an M2 having a nucleic acid sequence of SEQ ID NO: 42; or
an M1 having a nucleic acid sequence of SEQ ID NO: 33; and an M2 having a nucleic acid sequence of SEQ ID NO: 43.

48. The cassette of any one of claims 35-47, wherein the donor DNA sequence is a target strand donor DNA sequence.

49. A retron donor DNA-guide polynucleotide comprising a retron transcript comprising the product of the cassette of any one of claims 35-48, and optionally a gRNA.

50. The polynucleotide of claim 49, wherein the retron transcript and the gRNA are not linked together; or the retron transcript and the gRNA are linked together, such as part of the same transcript, or not part of the same contiguous transcript.

51. The polynucleotide of claim 50, wherein the retron and gRNA are covalently linked together.

52. The polynucleotide of any one of claims 49-51, wherein the retron transcript and the gRNA comprises a structured motif at a 5′ or a 3′ end of the retron-gRNA transcript, such as evoPreQ, tevopreQ mpKnot, or exoribonuclease-resistant RNA motif.

53. The polynucleotide of claim 52, wherein the structured motif comprises a nucleic acid sequence selected from any one of SEQ ID NO: 75, 76, 77, 78, 79, 80, 81, or 82.

54. The polynucleotide of any one of claims 49-53, wherein the retron transcript linked to the gRNA molecule comprises a formula A1-Rt-L1n-gRNA-A2, wherein:
(a) A1 is the structured motif, or is absent;
Rt is the retron transcript;
L1n is a nucleotide linker from about 5 to about 40, about 8 to about 35, about 9, or about 33 basepairs, or is absent; and
A2 is the structured motif, or is absent;
(b) A1 is the structured motif;
Rt is the retron transcript;
L1n is a nucleotide linker from about 5 to about 40, about 8 to about 35, about 9, or about 33 basepairs; and
A2 is absent; or
(c) A1 is absent;
Rt is the retron transcript;
L1 is a nucleotide linker from about 5 to about 40, about 8 to about 35, about 9, or about 33 base pairs; and
A2 is the structured motif.

55. A vector (e.g., plasmid, virus, and the like) comprising the cassette of any one of claims 35-48, or the polynucleotide of any one of claim 49-54.

56. A polypeptide having the formula of: of (N1)q-C1-L1-(N2)qq-R1-(N3)qqq, wherein C1 is a nuclease, L1 is a peptide linker, R1 is a RT protein, and N1 and N2 are each independently, a NLS sequence, and wherein q, qq, or qqq, are each independently, 0, 1, 2, or 3.

57. The polypeptide of claim 56, wherein the polypeptide has the formula of: 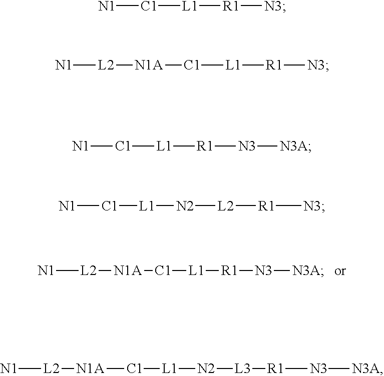
wherein:
each N1, N1A, N2, N3, or N3A is, independently, a NLS sequence;
each L1, L2, or L3 is independently, a linker amino acid sequence; and
wherein each NLS sequence is identical or different.

58. A polypeptide comprising a SSAP, RT, and a nuclease, wherein:
the C-terminus of the RT is linked to the N-terminus of the nuclease, and the C-terminus of the nuclease is linked to the N-terminus of the SSAP; or
the N-terminus of the RT is linked to the C-terminus of the nuclease, and the N-terminus of the nuclease is linked to the C-terminus of the SSAP.

59. A nucleic acid molecule encoding the polypeptide of any one of claims 56-58.

60. A vector comprising the nucleic acid molecule of claim 59, wherein the vector is a plasmid, virus, RNA, mRNA, and the like.

61. A cell comprising the cassette of any one of claims 35-48, the polynucleotide of any one of claim 49-54, or the polypeptide of any one of claim 56-58.

62. The cell of claim 61, wherein the cell is a mammalian cell, such as a human cell.

63. The cell of claim 61 or 62, wherein the is cell is in a subject.
